(19) United States
(12) Patent Application Publication
(10) Pub. No.: US 2017/0215940 A1
(43) Pub. Date: Aug. 3, 2017

(54) CONTROL AND DELIVERY OF ELECTRIC FIELDS VIA AN ELECTRODE ARRAY

(71) Applicant: LaZure Scientific, Inc., Issaquah, WA (US)

(72) Inventors: Charles E. Hill, Issaquah, WA (US); Joshua S. Hockett, Seattle, WA (US)

(21) Appl. No.: 15/376,438

(22) Filed: Dec. 12, 2016

Related U.S. Application Data
(63) Continuation of application No. 14/365,450, filed on Jun. 13, 2014, now abandoned.
(60) Provisional application No. 61/570,154, filed on Dec. 13, 2011.

(51) Int. Cl. A61B 18/12; A61B 18/14

(52) U.S. Cl. CPC A61B 18/1206; A61B 18/1477; A61B 2018/00547

(57) ABSTRACT

A method of controlling electric fields created by a plurality of electrodes. The method includes repetitively applying multiple sets of voltages to at least some of a plurality of electrodes over a treatment period to achieve and maintain a target temperature, the at least some of the electrodes being treatment electrodes. The sets of voltages may be in patterns such that a unique current pattern between electrodes is created for each set of voltages, resulting in temperature averaging. The voltage at each electrode may be determined based on a temperature of an adjacent electrode. The voltage at each electrode may also or alternatively be determined based on an estimated voltage at the electrode.

### CROSS-REFERENCE

This application is a Continuation of U.S. application Ser. No. 14/365,450, filed Jun. 13, 2014, which is a U.S. National Phase application under 35 U.S.C. §371 of International Application No. PCT/US12/69430, filed Dec. 13, 2012, which claims the benefit of U.S. Provisional Application No. 61/570,154, filed Dec. 13, 2011, the entirety of each of which is hereby incorporated by reference.

### BACKGROUND OF THE INVENTION

Embodiments of the present invention relate generally to controlling and delivering electric fields. More particularly, embodiments of the present invention provide systems and methods for controlling and delivering current to a tissue (e.g., prostate tissue) of a patient for the destruction of cancerous and/or hyperplastic cells or tissue.

The prostate gland is a walnut-sized gland located in the pelvic area, just below the outlet of the bladder and in front of the rectum. It encircles the upper part of the urethra, which is the tube that empties urine from the bladder. The prostate is an important part of the male reproductive system, requiring male hormones like testosterone to function properly, and helps to regulate bladder control and normal sexual functioning. The main function of the prostate gland is to store and produce seminal fluid, a milky liquid that provides nourishment to sperm and increases sperm survival and mobility.

Cancer of the prostate is characterized by the formation of malignant (cancerous) cells in the prostate. Prostate cancer is the leading cancer-related cause of death in men in the United States. There are currently over 2 million men in the United States with prostate cancer, and it is expected that there will be approximately 190,000 new cases of prostate cancer diagnosed, with 28,000 men dying from the disease in 2008.

In addition to risks of morbidity due to prostate cancer, most men over 60 years old experience partial or complete urinary obstruction due to enlargement of the prostate. This condition can originate from prostate cancer, or more typically, from benign prostatic hyperplasia (BPH), which is characterized by an increase in prostate size and cell mass near the urethra.

Common active treatment options include surgery and radiation. Surgery often includes the complete surgical removal of the prostate gland (“Radical Prostatectomy”), and in certain instances the regional lymph nodes, in order to remove the diseased tissue from the body. In some instances, a nerve sparing prostatectomy is attempted in an effort to maintain erectile function in the patient after treatment. Side effects associated with radical prostatectomy can include pain, inflammation, infection, incontinence, shorter penis and impotence.

Radiation therapy is another treatment option for prostate cancer and is characterized by the application of ionizing radiation to the diseased area of the prostate. Ionizing radiation has the effect of damaging a cells DNA and limiting its ability to reproduce. For prostate cancer treatment, two methods of radiation therapy include External Beam Radiation Therapy (EBRT) and internal radiation, commonly known as Brachytherapy. EBRT involves the use of high-powered X-rays delivered from outside the body. The procedure is painless and only takes a few minutes per treatment session, but needs to be over extended periods of five days a week, for about seven or eight weeks. During EBRT, the rays pass through and can damage other tissue on the way to the tumor, causing side effects such as short-term bowel or bladder problems, and long-term erectile dysfunction. Radiation therapy can also temporarily decrease energy levels and cause loss of appetite.

Brachytherapy involves the injection of a tiny radioactive isotope containing ‘seeds’ into the prostate. Once positioned in the tissue, the radiation from the seeds extends a few millimeters to deliver a higher radiation dose in a smaller area, causing non-specific damage to the surrounding tissue. The seeds are left in place permanently, and usually lose their radioactivity within a year. Internal radiation also causes side effects such as short-term bowel or bladder problems, and long-term erectile dysfunction. Internal radiation therapy can also temporarily decrease energy levels and cause loss of appetite. It is also common for the implanted seeds to migrate from the prostate into the bladder and then be expelled through the urethra during urination. Most significant, however, is the change in the texture of the prostate tissue over time, making the subsequent removal of the gland, as described above, complicated and difficult as a secondary treatment.

Given the significant side-effects with existing treatments such as radical prostatectomy and radiation therapy, less invasive and less traumatic systems and procedures have been of great interest. One such more minimally invasive system developed in recent years includes so called “Trans-urethral Needle Ablation” or TUNA, which involves passing a radio-frequency (RF) device such as a catheter electrode or scope into the urethra for delivery of high frequency energy to the tissue. The RF instruments include electrode tips that are pushed out from the side of the instrument body along off-axis paths to pierce the urethral wall and pass into the prostatic tissue outside of the urethra. High-frequency energy is then delivered to cause high-temperature ionic agitation and frictional heating to tissues surrounding the electrodes. The high temperature induced in the tissue, e.g., up to 90-100 degrees C. or more, is non-specific to cancerous tissue and destroys both healthy and non-healthy tissue.

Another technique developed in recent years for treating BPH is Trans-urethral Microwave Thermo Therapy (or “TUMT”). This technique involves use of a device having a microwave electrode or antenna located near its distal end and connected to an external generator of microwave power outside the patient's body. The microwave electrode is inserted into the urethra to the point of the prostate for energy delivery and microwave electromagnetic heating. Since the microwave electrode delivers substantial heating that can cause unwanted damage to healthy tissues or to the urethra, devices typically make use of a cooled catheter to reduce heating immediately adjacent to the electrode. The objective is to carefully balance cooling of the urethra to prevent damage to it by the heating process, while at the same time delivering high temperature heating (typically much greater than 50 degrees C.) to the prostatic tissue outside of and at a distance from the urethra. In this procedure, the prostatic tissue immediately around the urethra and the urethra itself are deliberately spared from receiving an ablative level of heating by attempting to keep the temperatures for these structures at less than 50 degrees C. Unfortunately, controlling the tissue heating due to the applied microwave energy is difficult and unintended tissue damage can occur. Further, destruction of tissue beyond the cooled region is indiscriminate, and control of the treatment zone is imprecise and limited in the volume of tissue that can be effectively treated.

Accordingly, there is a continuing interest to develop less invasive devices and methods for the treatment of cancerous or hyperplastic conditions, such as in BPH and prostate cancer, that is more preferential to destruction of hyperplastic/cancerous cells of target tissue and more precisely controllable.

### BRIEF SUMMARY OF THE INVENTION

Embodiments of the present invention include a method of controlling electric fields created by a plurality of electrodes. The method includes repetitively applying multiple sets of voltages to at least some of a plurality of electrodes over a treatment period so as to heat a target area (e.g., an area or volume of a target tissue) to a selected or desired temperature or temperature range. At least some electrodes may be treatment electrodes. The multiple sets of voltages may include a first set of voltages that creates an electric potential difference between at least some adjacent pairs of the treatment electrodes; and a second set of voltages that creates an electric potential difference between at least some adjacent pairs of the treatment electrodes for which an electric potential difference was not created while applying the first set of voltages. In one embodiment, the multiple sets of voltages in combination create an electric potential difference between each adjacent pair of treatment electrodes.

Embodiments of the present invention also include a system for selectively generating electric fields. The system includes a plurality of electrodes and a control unit, where the control unit may include a storage medium and a computer processor, the storage medium having executable instructions stored thereon. The computer processor may be operable to execute the instructions so as to cause the control unit to perform operations including switching between different or unique electrode patterns, where each unique electrode pattern includes providing an electrical voltage to at least some of the electrodes, the at least some electrodes being treatment electrodes, and the electrical voltage being provided so as to generate a current flow between adjacent pairs of the treatment electrodes. The operations may further include applying a feedback control loop controlling the electrical voltage provided to the treatment electrodes based at least in part on one or more of: a temperature difference for a treatment electrode based on a temperature of an adjacent treatment electrode, and an estimate of a voltage at a treatment electrode provided by one or more other treatment electrodes.

Embodiments of the present invention further include a control unit for controlling electric fields created by a plurality of electrodes. The control unit may include a storage medium and a computer processor, the storage medium having executable instructions stored thereon. The computer processor may be operable to execute the instructions so as to cause the control unit to perform operations including applying a feedback control loop controlling an electrical voltage provided to at least some of a plurality of electrodes, the at least some electrodes being treatment electrodes. Wherein applying a feedback control loop may include, for each treatment electrode, adjusting a voltage applied to the electrode based at least in part on one or more of: a temperature difference for the electrode based on a temperature of an adjacent electrode, and an estimate of a voltage at the electrode provided by one or more other electrodes.

For a fuller understanding of the nature and advantages of the present invention, reference should be made to the ensuing detailed description and accompanying drawings. Other aspects, objects and advantages of the invention will be apparent from the drawings and detailed description that follows.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A illustrates a simplified system for selectively applying electric fields to target areas in accordance with an embodiment.

FIG. 1B illustrates a simplified system control unit for controlling a needle electrode assembly according to an embodiment.

FIG. 2A is a profile view of an electrode assembly according to an embodiment.

FIG. 2B is a top view of the electrode assembly of FIG. 2A with electrodes disengaged from a housing.

FIG. 2C is a first side view of the electrode assembly of FIG. 2A.

FIG. 2D is a second side view of the electrode assembly of FIG. 2A.

FIG. 2E is a third side view of the electrode assembly of FIG. 2A.

FIG. 2F is a top view of the electrode assembly of FIG. 2A with electrodes engaged with a housing.

FIG. 3A is a profile view of an electrode according to an embodiment.

FIG. 3B is a cross-sectional view of the electrode of FIG. 3A.

FIG. 4A is a profile view of an electrode guide according to an embodiment.

FIG. 4B is a front view of the electrode guide of FIG. 4A.

FIG. 4C is a side view of the electrode guide of FIG. 4A.

FIG. 4D is a top view of the electrode guide of FIG. 4A.

FIG. 5A is a profile view of a template according to an embodiment.

FIG. 5B is a front view of the template of FIG. 5A.

FIG. 5C is a cross sectional view of the template of FIG. 5A.

FIG. 6 is a flowchart depicting example operations of a method for controlling a position of one or more elongated electrodes.

FIG. 7A shows a user interface for monitoring and controlling a plurality of electrodes according to an embodiment.

FIG. 7B shows a treatment parameter element of the user interface of FIG. 7A.

FIG. 7C shows a patient information element of the user interface of FIG. 7A.

FIG. 7D shows an electrode control element of the user interface of FIG. 7A.

FIG. 7E shows an electrode status element of the user interface of FIG. 7A.

FIG. 7F shows a magnified portion of the electrode status element of FIG. 7E.

FIG. 8 is a flowchart depicting example operations of a method for controlling electric fields created by a plurality of electrodes according to an embodiment.

FIG. 9 is flowchart depicting example operations of a method for performing pattern switching according to an embodiment.

FIG. 10A shows a first electrode pattern of a set of electrode patterns and the resulting current flow pattern according to an embodiment.

FIG. 10B shows a second electrode pattern of a set of electrode patterns and the resulting current flow pattern according to an embodiment.

FIG. 10C shows a third electrode pattern of a set of electrode patterns and the resulting current flow pattern according to an embodiment.

FIG. 11A shows AC signals for generating a difference in electric potential based on a difference in signal polarity or phase.

FIG. 11B shows AC signals for generating a difference in electric potential based on a difference in signal amplitude.

FIG. 11C shows AC square wave signals for generating a difference in electric potential based on a pulse width modulation of the signals.

FIG. 12 is a flowchart depicting example operations of a customized feedback control process according to a first embodiment.

FIG. 13A is a flowchart depicting example operations of a customized feedback control process according to a second embodiment.

FIG. 13B is a flowchart depicting example operations for setting an electrode temperature in accordance with operation 1340 of FIG. 13A.

FIG. 13C is a flowchart depicting example operations for modifying a voltage of an electrode in accordance with operation 1360 of FIG. 13A.

FIG. 14A shows the voltages and temperatures of a plurality of electrodes for a time instance in which a first electrode pattern is applied.

FIG. 14B shows the voltages and temperatures of a plurality of electrodes for a time instance in which a second electrode pattern is applied.

FIG. 14C shows the voltages and temperatures of a plurality of electrodes for a time instance in which a third electrode pattern is applied.

FIG. 14D shows the voltages and temperatures of a plurality of electrodes for another time instance in which the first electrode pattern is applied.

FIG. 14E shows the voltages and temperatures of a plurality of electrodes for another time instance in which the second electrode pattern is applied.

FIG. 14F shows the voltages and temperatures of a plurality of electrodes for another time instance in which the third electrode pattern is applied.

FIG. 15A illustrates a mobile cart including one or more components for selectively applying electric fields to target areas in accordance with an embodiment.

FIG. 15B illustrates a cassette-based needle electrode assembly according to an embodiment.

FIG. 15C illustrates a controller according to an embodiment.

FIG. 15D illustrates a cassette rack for receiving one or more cassettes.

FIG. 16 shows a method for facilitating treatment of a target area.

FIG. 17A shows a user interface for displaying a configuration prompt according an embodiment.

FIG. 17B shows a user interface for a loaded cassette.

FIG. 17C shows the user interface of FIG. 17B with a user-selected cassette electrode.

FIG. 17D shows the user interface of FIG. 17C with a user selected cassette electrode having been placed at a node of a grid array.

FIG. 17E shows a user interface 1710 in which a plurality of electrodes from two cassettes have been placed.

FIG. 17F shows the user interface of FIG. 17E after treatment has begun.

FIG. 17G shows the user interface of FIG. 17F upon completion of a treatment.

### DETAILED DESCRIPTION OF THE INVENTION

Embodiments of the present invention provide systems, devices, and methods for selectively monitoring and controlling electric fields. For example, voltages applied to electrodes and/or current and heating to the target tissues may be selectively controlled, and temperatures in regions proximate to the electrodes can be selectively monitored. In some embodiments, the electrodes may be introduced into a target tissue region and an electric field applied to the target tissue region for controlled and/or preferential destruction of cancerous and/or hyperplastic cells of the target tissue compared to non-cancerous or non-hyperplastic cells in the treatment region.

In some embodiments, tissue heating may be performed using a plurality of electrodes disposed in a treatment area. Voltages may be applied to the electrodes in a plurality of voltage patterns, where voltages applied to the electrodes may be changed so as to switch between the voltage patterns. By applying voltages to the electrodes using a number of voltage patterns, current densities and thus electrode temperatures may be averaged out over all of the electrodes, thereby reducing the number and/or effect of localized hot spots.

In other embodiments, the voltage to be applied to each electrode may be determined using a customized feedback control loop. The customized feedback control loop may determine a temperature difference for a controlled electrode based on a temperature of an adjacent electrode. By using a temperature of an adjacent electrode, the voltage of the controlled electrode may be controlled so as to prevent an overheating of the adjacent electrode. In some cases, the customized feedback control loop may estimate an average voltage provided at the controlled electrode from other electrodes, and use this average voltage in determining the voltage to apply to the controlled electrode. By using an average voltage provided at the controlled electrode from other electrodes, such as adjacent electrodes, a current flow to or from the controlled electrode may be more accurately controlled. These and other embodiments are further described herein.

System for Applying Electric Fields

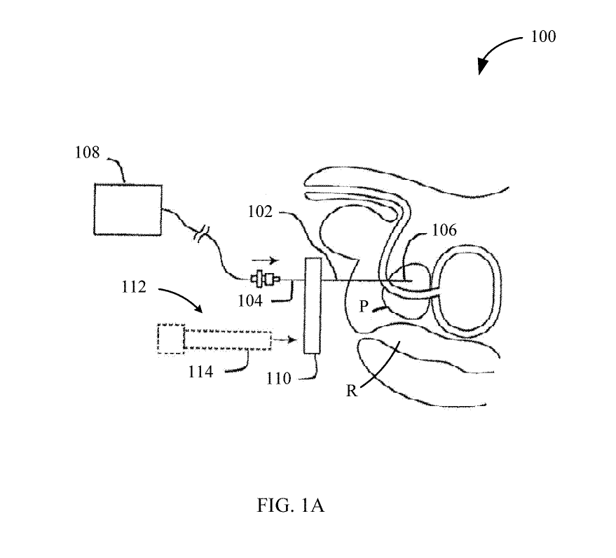

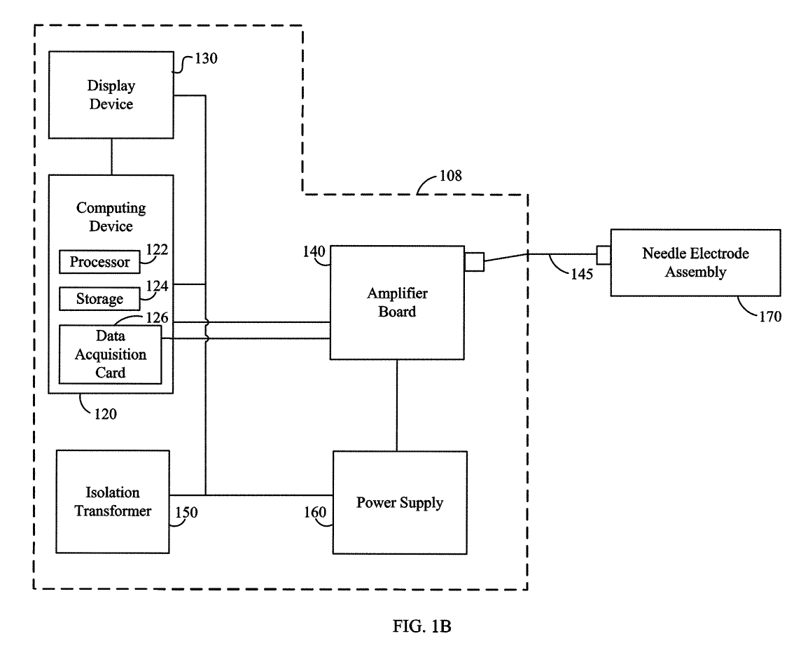

FIG. 1A illustrates a simplified system 100 for selectively applying electric fields to target areas in accordance with an embodiment. System 100 includes a plurality of elongated electrodes, such as electrode 102, having a proximal portion 104 and a distal portion 106. The distal portion 106 includes a portion configured for delivery of an electrical field when positioned in the prostate tissue (P). The electrode can be advanced through the skin and through the perineum of a patient so that the distal portion is positioned in the target area (e.g., prostate tissue (P)) of the patient. The proximal portion 104 of the electrode 102 is electrically connected to a system control unit 108, as above, which can include electronics, storage media, programming, etc., as well as a power unit, for controlled delivery of selected electrical fields to the target tissue. As illustrated, the system 100 can optionally include an electrode guide 110 for controlled placement and positioning of the electrode 102 in the tissue of the patient. The system 100 can further include an imaging device/system 112, which can include imaging systems which may be used for guidance and placement of the electrode 102. For example, the imaging device 112 can include a distal portion 114 including electronics and imaging components (e.g., ultrasonic scanning transducer), which can be inserted in a patient's rectum (R) and positioned against the rectal wall near the prostate (P). An exemplary imaging device 112 can include those commonly used for diagnostic medicine, such as commercially available ultrasonic imaging devices. The electrode guide 110 can optionally be designed for coupling with the imaging device 112, such that electrode guide 110 and the imaging device 112 form a single stable assembly.

Some general features and functionality of certain system 100 aspects or components may be described in U.S. patent application Ser. Nos. 12/251,242, 12/283,847, 12/761,915, which are commonly assigned and incorporated herein by reference in their entirety.

System 100 in certain embodiments is a system for selectively applying electric fields to target tissues including various components such as an electrode 102, a system control unit 108, a electrode guide 110, and an imaging device/system 112. However, it will be appreciated by those of ordinary skill in the art that such a system could operate equally well in a system having fewer or a greater number of components than are illustrated in FIG. 1A. Thus, the depiction of system 100 in FIG. 1A should be taken as being illustrative in nature, and not limiting to the scope of the disclosure.

System Control Unit

FIG. 1B illustrates a simplified system control unit 108 for controlling a needle electrode assembly 170 according to an embodiment. System control unit 108 may include one or more elements, such as a computing device 120, a display device 130, an amplifier board 140, an isolation transformer 150, and a power supply 160 (e.g., a DC power supply). System control unit 108 may be the same as that discussed with reference to FIG. 1A, and needle electrode assembly 170 may include one or more electrodes such as electrodes 102 discussed with reference to FIG. 1A. Accordingly, in some embodiments, system control unit 108 may control electrodes in electrode assembly 170 to deliver electric fields to tissue for tissue ablation. Further, as discussed herein, system control unit 108 may optionally also utilize thermistors provided within needle electrode assembly 170 to monitor temperatures; e.g., temperatures of tissue in regions proximate to the electrodes.

Computing device 120 may include, e.g., a computer or a wide variety of proprietary or commercially available computers or systems having one or more processing structures, a personal computer, and the like, with such systems often comprising data processing hardware and/or software configured to implement any one (or combination of) the processing operations described herein. Any software will typically include machine readable code of programming instructions embodied in a non-transitory tangible media such as an electronic memory, a digital or optical recovering media, or the like, and one or more of these structures may also be used to transmit data and information between components of the system in any wide variety of distributed or centralized signal processing architectures. According to one embodiment, computing device 120 includes a single core or multi-core processor 122 and a tangible non-transitory computer-readable storage device 124, where processor 122 may execute computer-readable code stored in storage device 124.

Display device 130 may be any type of suitable device for displaying information to an operator of system control unit 108. For example, display device 130 may incorporate cathode ray tubes, liquid crystals, light emitting diodes, electrically charged ionized gases (i.e., a plasma display), and the like. In some embodiments, system control unit 108 may further include one or more input devices (not shown), such as a mouse, keyboard, keypad, trackball, light pen, and the like. Such input devices may be electrically coupled to computing device 120 to enable the operator of system control unit 108 to provide inputs to computing device 120. In other embodiments, display device 130 may additionally or alternatively enable the operator of system control unit 108 to provide inputs to computing device 120. For example, display device 130 may comprise a touchscreen display.

Display device 130 is in communication with computing device 120 to enable data to be transferred between the two devices. For example, display device 130 may be electrically coupled to computing device 120 via a connecting cable. For another example, display device 130 and computing device 120 may communicate data to one another wirelessly over any suitable wireless communication protocol, such as Bluetooth™, IEEE 802.11, etc.

Amplifier board 140 may be any suitable amplifier for driving one or more electrodes in a needle electrode assembly 170 and/or receiving and communicating temperature measurements from needle electrode assembly 170. In some embodiments, amplifier board 140 is operable to individually control at least one of a voltage and current amplitude and phase applied to each of the electrodes of electrode assembly 170. Amplifier board 140 may be operable to sample at least one of a voltage, current, and temperature of each of the electrodes. Amplifier board 140 may also be operable to electrically disconnect one or more of the electrodes, connect one or more of the electrodes to ground, or connect one or more of the electrodes to a driving signal. For example, amplifier board 140 may include, for each electrode, a relay for controlling a state of the electrode. In one embodiment, amplifier board 140 performs signal conditioning on at least one of voltage, current, and temperature measurements sampled from electrode assembly 170.

Amplifier board 140 may be electrically coupled to needle electrode assembly 170 via, for example, a cable assembly 145. Cable assembly 145 may enable communication between amplifier board 140 and needle electrode assembly 170, and may enable amplifier board 140 to provide power to electrodes of needle electrode assembly 170. According to one embodiment, electrode assembly 170 includes thermistor circuitry for calculating temperatures of the electrodes. In such a case, amplifier board 140 may route signals from the thermistor circuitry to computing device 120 and supply power to the thermistor circuitry. According to other embodiments, other devices may be capable of calculating temperatures of the electrodes. For example, computing device 120 may perform such calculations based on measurements received from electrodes in electrode assembly 170.

Computing device 120 may also include a data acquisition card 126. Data acquisition card 126 may be electrically or wirelessly coupled to amplifier board 140 and may receive various measurement data read by amplifier board 140. For example, data acquisition card 126 may receive voltage, current, and temperature measurements of each of the electrodes. In some embodiments, data acquisition card 126 may receive such measurements after amplifier board 140 has performed signal conditioning.

According to some embodiments, data acquisition card 126 may further be configured to control amplifier board 140. For example, data acquisition card 126 may provide a digital bit stream to amplifier board 140 instruct amplifier board 140 to drive one or more of the electrodes and acquire various measurements. In some embodiments, the digital bit stream may be clocked into memory of amplifier board 140 and, as a result, field programmable gate arrays (FPGAs) of amplifier board 140 may configure various subcomponents of amplifier board 140 to output and measure the desired signals. This may be done many times per second, allowing for smooth, closed-loop control of the system.

Isolation transformer 150 may be any suitable transformer for transferring alternating current (AC) power from an AC power source to one or more other elements of the system, such as computing device 120, display device 130, and direct current (DC) power supply 160, while isolating such elements from the earth ground.

DC power supply 160 may be any suitable power supply for converting AC power to DC power. In some embodiments, DC power supply 160 converts AC power received from isolation transformer 150 to DC power and provides the DC power to amplifier board 140. In other embodiments, amplifier board 140 includes an AC/DC converter and receives AC power directly or uses battery power, thus obviating the need for DC power supply 160.

Needle electrode assembly 170 may be electrically coupled to amplifier board 140 as previously discussed. Needle electrode assembly 170 includes a plurality of needle or elongated electrodes. The electrodes may each generate an electric field based on a voltage and current provided by amplifier board 140. In some embodiments, one or more electrodes may include or be replaced by a thermistor for measuring a temperature of the electrodes or within a vicinity of the electrodes. In some cases, one or more electrodes are used for measuring temperature, but not for generating an electric field. For example, some electrodes may be used to monitor temperature and provide a reference temperature (e.g., a body temperature).

According to one embodiment, the electrodes may be individually advanced and positioned within a target tissue (e.g., a prostate tissue). Once the electrodes are positioned, a voltage can be applied to one or more of the electrodes, thereby causing electrical fields, magnetic fields, and currents to be generated in portions of the target tissue. Such fields may be used, for example, for tissue ablation to destroy cancerous and/or hyperplastic cells.

System control unit 108 in certain embodiments is a system for controlling a needle electrode assembly including various components such as a computing device 120, a display device 130, an amplifier board 140, an isolation transformer 150, and a DC power supply 160. However, it will be appreciated by those of ordinary skill in the art that the system control unit could operate equally well by having fewer or a greater number of components than are illustrated in FIG. 1B. Thus, the depiction of system control unit 108 in FIG. 1B should be taken as being illustrative in nature, and not limiting to the scope of the disclosure.

Electrode Assembly

A system will typically include a plurality or array of electrodes that operatively couple to one or more components of a system (e.g., power source, etc) and can be positioned in the tissue for delivery of current field as described herein. Some of all of the electrodes may be used for delivery of a current field. For example, a plurality of electrodes may be positioned in the tissue, but only some of those electrodes used for delivery of a current field. Various different electrode configurations and assemblies can be utilized and may be suitable for current field delivery as described herein.

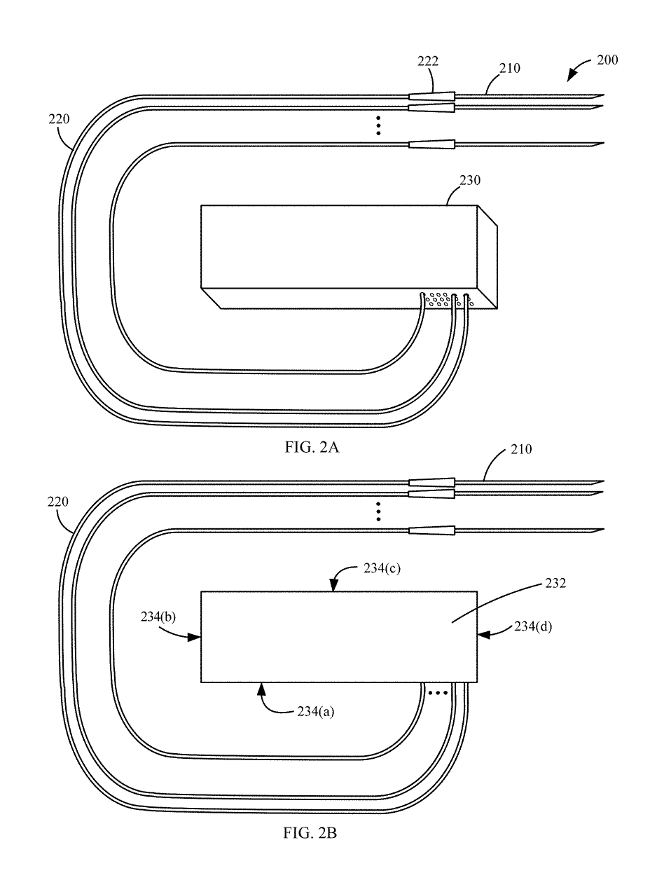

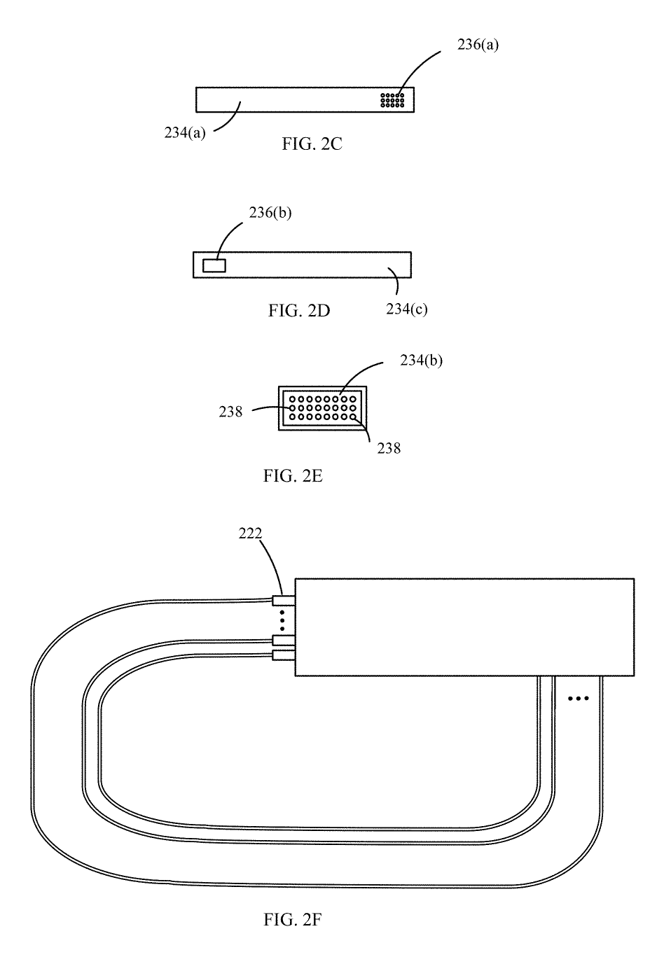

FIG. 2A is a profile view of an exemplary electrode assembly 200 according to an embodiment. Electrode assembly 200 includes a plurality of elongated electrodes 210, a plurality of flexible conductive wires 220, and a housing 230.

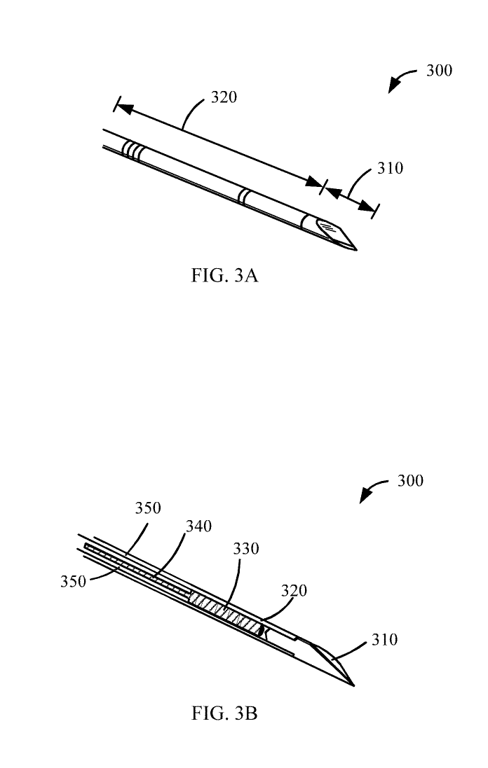

In one embodiment, elongated electrodes 210 are substantially cylindrical in shape. A distal end of elongated electrodes 210, for example, an end for penetrating tissue of a patient, is narrowed to a tip. Such narrowing may advantageously reduce penetration resistance when inserting the electrode into an object such as tissue of a patient. A proximal end of elongated electrodes 210 may be mechanically and electrically coupled to an end of a corresponding conductive wire 220. Accordingly, current, voltage, and/or temperature measurements may be communicated to and from electrodes 210 via the conductive wires 220. In other embodiments, elongated electrodes 210 may have other shapes, such as being elongated with a square, rectangular, or oval cross-section. In some embodiments, elongated electrodes 210 have a variety or a combination of shapes. Electrodes 210 are further discussed with reference to FIGS. 3A to 3B.

Each of the plurality of wires 220 includes a first end and a second end, where the first end is mechanically coupled to one of electrodes 210 and the second end is mechanically coupled to an interface of housing 230. Each wire 220 may comprise one or more cores that may be made of any suitable conductive material, such as copper, aluminum, metal alloys, coated metals, etc. In case each wire comprises only a single core, the single core may be insulated with a non-conductive sheath made of any suitable insulating material, such as plastic, silk, etc. In case each wire comprises a plurality of conductive cores, each core may be insulated, and the plurality of insulated cores may then be bundled with, e.g., a further sheath. In some embodiments, the further sheath may also be made of non-conductive material.

In one embodiment, one or more of wires 220 includes a shielding element. The shielding element is operable to prevent EMI leakage and noise on measured signals. The shielding element may be made of any suitable material and may include, for example, a braided or foil-type shielding.

Each core may be operable to communicate any suitable signal or signals to and/or from electrodes 210. For example, each core may communicate electrical voltage, resistance, and/or current, and/or differences in voltage, resistance, and/or current, etc. This may include treatment signals, which may be any suitable signal for excising tissue, and may include temperature measurement signals, which may be any suitable signal for measuring a temperature in, on, or around electrodes 210.

The first end of each wire 220 may include an enlarged portion 222. The enlarged portion may be of any suitable shape. In one embodiment, enlarged portion 222 has a cross-section having a shape that is the same shape as at least a portion of electrode 210. In another embodiment, enlarged portion 222 has a cross-section having a shape this is the same shape as wire 220. For example, enlarged portion 222 may have a cross-section in the shape of a circle, oval, rectangle, etc. Enlarged portion 222 may be enlarged such that a diameter of enlarged portion 222 is larger than a diameter of other portions of wire 222. The diameter of enlarged portion 222 may be stay the same along a length of enlarged portion 222, or may vary along the length of enlarged portion 222. In one embodiment, the diameter of enlarged portion 222 at an end proximate to electrode 210 is larger than the diameter of enlarged portion 222 at an end proximate to other portions of wire 210. In another embodiment, enlarged portion 222 may include a surface proximate to electrode 210 that is planar and perpendicular to a direction in which electrode 210 extends. In some embodiments, enlarged portion 222 may be part of electrode 210 rather than wire 220.

Enlarged portion 222 may serve one or more functions. In one embodiment, enlarged portion 222 may provide a location that is easy to grasp by a clinician. In a further embodiment, enlarged portion may protect and insulate connections between conductive cores of a wire 220 and portions of an electrode 210. In another embodiment, enlarged portion 222 may provide a depth stop for electrodes 210. For example, where a electrode guide 110 includes a plurality of apertures to allow electrodes 210 to pass through, the enlarged portions 222 may be sized so that they abut the electrode guide 110 and prevent electrodes 210 from passing entirely through the electrode guide 110. In one embodiment, enlarged portions 222 may have a diameter larger than a diameter of receiving apertures of electrode guide 110. In another embodiment, enlarged portion 222 may have a cross section having a shape that is different than a shape of a receiving aperture of electrode guide 110.

Housing 230 selectively receives the plurality of electrodes 210 and includes an interface for providing an electrical coupling to electrodes 210. Housing 230 may further include apertures for receiving the plurality of electrodes 210, and electronics for calculating thermal measurements, passing voltages and currents to electrodes 210, and the like. The interface may include a first interface portion mechanically coupled to wires 220, and a second interface portion for receiving a cable assembly from amplifier board 140 such as cable assembly 145.

Housing 230 may be any suitable shape. For example, as illustrated in FIG. 2A, housing 230 may have substantially rectangular cross sections. For another example, housing 230 may have substantially square, circular, or oval cross sections, or cross sections of any other suitable shape. Housing 230 may be made of any suitable material. For example, housing 230 may be made of organic solids such as polymers, composite materials such as thermoplastic matrices, metals, ceramics, etc.

FIG. 2B is a top view of the electrode assembly of FIG. 2A with electrodes disengaged from a housing. According to one embodiment, housing 230 includes a top surface 232, a bottom surface (not shown), and side surfaces 234(a) to 234(d). In this embodiment, top surface 232 and side surfaces 234(a) to 234(d) are substantially planar and are substantially perpendicular to one another. However, in other embodiments, such surfaces may be curved or angled, and provided at angles other than 90 degrees. Further, wires 220 are mechanically coupled to side surface 234(a). However, in other embodiments, wires 220 may be mechanically coupled to other surfaces, such as the top surface, the bottom surface, and the like.

FIG. 2C is a first side view of the electrode assembly of FIG. 2A. According to one embodiment, housing 230 includes a first portion of an interface 236(a) for mechanically coupling to wires 220. Interface portion 236(a) includes conductive components such that the mechanical coupling provides an electrical coupling to wires 220 and electrodes 210. According to this embodiment, interface portion 236(a) is provided on side 234(a). However, according to other embodiments, interface portion 236(a) may be provided on any of the other surfaces of housing 230.

FIG. 2D is a second side view of the electrode assembly of FIG. 2A. According to one embodiment, housing 230 includes a second portion of an interface 236(b) for mechanically coupling to a cable such as cable assembly 145. Interface portion 236(b) includes conductive components such that the mechanical coupling provides an electrical coupling to conductive cores in cable assembly 145. According to this embodiment, interface portion 236(b) is provided on side 234(c). However, according to other embodiments, interface portion 236(b) may be provided on any of the other surfaces of housing 230.

As previously mentioned, housing 230 may include electronics for calculating thermal measurements, passing voltages and currents to electrodes 210, and the like. For example, housing 230 may surround or embody a printed circuit board (PCB) having circuitry and/or software for calculating thermal measurements from electrodes 210. The PCB may be partially or fully disposed, mechanically and electrically, between interface portion 236(a) and interface portion 236(b). Housing 230 may also include electronics for storing data. Stored data may include identification data such as a serial number, mode number, expiration data, authentication code, etc. In some embodiments, such stored data may be read by various computing devices, such as computing device 120.

FIG. 2E is a third side view of the electrode assembly of FIG. 2A. According to one embodiment, housing 230 includes a plurality of apertures 238 for receiving the plurality of electrodes 210. Apertures 238 may each have a shape corresponding to an electrode 210. For example, apertures 238 may protrude into a depth of housing 230 and have a substantially circle cross-section. However, apertures 238 may have other shapes as well, such as rectangular, square, or oval cross-sections. In some embodiments, apertures 238 may be spaced apart from one another so as to electrically insulate electrodes 210 from one another when housing 230 receives electrodes 210. According to one embodiment, apertures 238 are provided on side 234(b). However, according to other embodiments, apertures 238 may be provided on any of the other surfaces of housing 230.

FIG. 2F is a top view of the electrode assembly of FIG. 2A with electrodes engaged with a housing. By engaging the electrodes into the housing, the electrodes may advantageously be protected during transportation, and the electrode assembly may advantageously be handled after sterilization of the electrodes.

According to one embodiment, housing 230 may receive electrodes 210 via apertures 238. Housing 230 may use any suitable mechanism for maintaining electrodes 210 within apertures 238 so as to advantageously reduce the likelihood of electrodes 210 unexpectedly disengaging from apertures 238. For example, electrodes 210 may have a friction fit with apertures 238. Upon engaging electrodes 210 with apertures 238, enlarged portions 222 of wires 220 may extend from a side surface of housing. In one embodiment, apertures 238 and enlarged portions 222 may be sized to create a friction fit between enlarged portions 222 and apertures 238.

Electrode assembly 200 in certain embodiments is an assembly of electrodes for generating electric fields so as to create current patterns in a delivery medium, and may include various components such as elongated electrodes 210, flexible conductive wires 220, and a housing 230. However, it will be appreciated by those of ordinary skill in the art that the electrode assembly could operate equally well by having fewer or a greater number of components than are illustrated in FIGS. 2A to 2F. Thus, the depiction of electrode assembly 200 in FIGS. 2A to 2F should be taken as being illustrative in nature, and not limiting to the scope of the disclosure.

For example, in some embodiments, electrode assembly 200 may consist only of elongated electrodes 210. In such cases, electrodes 210 may be controlled by electrode guide 110, and/or information may be communicated to and from the electrodes via electrode guide 110. For example, receiving apertures of electrode guide 110 may each include electrical contacts for electrically contacting a received electrode. The electrical contacts may then operate to communicate current to and/or from received electrodes. The electrical contacts may be powered and/or in wired or wireless communication with other parts of system 100, such as system control unit 108, so as to facilitate power transfer and/or information communication between electrodes 210 and system control unit 108. In some embodiments, electrodes 210 may include circuitry such as a wireless communication interface and/or a power supply, so that electrodes 210 may be in wireless communication with parts of system 100 such as system control unit 108 and/or may communicate current to and/or from a target area regardless of whether electrode guide 110 includes elements for controlling and/or powering electrodes 210.

FIG. 3A is a profile view of an electrode 300 according to an embodiment. Electrode 300 includes an exposed portion 310 and an insulated portion 320. Exposed portion 310 includes a conductive surface and a sharpened point, and is operable to deliver a treatment signal to tissue. The exposed portion 310 may be made of any suitable conductive material, such as copper, aluminum, metal alloys, coated metals, etc. In some embodiments, the exposed portion 310 of an electrode may be operable to conduct current to another electrode or conductive entity, so as to generate heat by way of the current path. In other embodiments, the exposed portion 310 of an electrode may be operable to generate heat itself, so that the heat generated is localized to the exposed portion 310. For example, the exposed portion 310 may be made of any suitable resistive material, such as carbon, carbon composites, metal, coated metal, metal-oxide, etc. Insulated portion 320 includes a non-conductive surface. The insulated portion 320 may be made of any suitable non-conductive material and, in some embodiments, may include a sheath wrapped around other parts of electrode 300, where the sheath is made of any suitable non-conducive material. For example, a heat shrink sleeve may be applied to the entire electrode 300 except for the exposed portion 310. The heat shrink sleeve may be made of, e.g., a polymer.

Exposed portion 320 may have any suitable length. For example, exposed portion 320 may have a length equal to 1 cm, 2 cm, 3 cm, or in a range between 1 cm and 3 cm, or less than 1 cm, or greater than 3 cm. The distance from the sharpened tip of electrode 300 may be indicated on an exterior surface of electrode 300. For example, distances of 1 cm, 2 cm, 3 cm, etc. may be marked on the surface of electrode 300. The indications may be made using any suitable method, such as chemical marking, laser marking, a printing process, etc.

Electrode 300 may have any suitable shape, size, and/or diameter, and electrode design or configuration may be selected based on the particular use of the system or aspects of a particular treatment to be performed. For example, electrode 300 may have a diameter of approximately 18 gauge, or a diameter in the range of 16 gauge to 20 gauge, or lower than 16 gauge or higher than 20 gauge. Electrode 300 may have any suitable length. For example, electrode 300 may have a length of approximately 20 cm, or a length in the range of 15 cm to 25 cm, or less than 15 cm or greater than 25 cm. According to one embodiment, electrode 300 is in the shape of a brachytherapy-style needle. According to other embodiments, electrode 300 is in a shape other than a needle, such as a catheter.

FIG. 3B is a cross-sectional view of the electrode of FIG. 3A. From the cross-sectional view, various components of an electrode according to one embodiment as visible. According to this embodiment, electrode 300 includes an exposed portion 310, insulated portion 320, a temperature sensor 330, temperature sensor lead 340, and electrode leads 350. From this perspective, it is apparent than in this embodiment, insulated portion 320 may form a shell or outer coating for other elements of electrode 300 to be arranged in. Exposed portion 320 includes a sharpened portion extending from insulated portion 320, and also includes a supporting portion that extends into insulated portion 320.

Temperature sensor 330 is operable to measure a temperature of or proximate to electrode 300. Temperature sensor 330 may be any suitable element for measuring temperature. For example, temperature sensor 330 may be a thermistor, thermocouple, resistive thermal device (RTD), etc. Temperature sensor 330 may be made of any suitable material. For example, sensor 330 may be made of platinum, platinum-covered ceramic, wire, glass-covered wire, one or more alloys, metals, etc. In this embodiment, temperature sensor 330 is arranged beside exposed portion 310. In one embodiment, electrode 300 includes a plurality of temperature sensors 330, either of the same or different type.

Electrode 300 includes one or more sensor leads 340 for communicating signals from sensor 330. Sensor lead 340 may be mechanically and electrically coupled to one or more cores of a wire 220. Sensor lead 340 may communicate any suitable signal from sensor 330. For example, sensor lead 340 may communicate electrical voltage, resistance, and/or current, and/or differences in voltage, resistance, and/or current, etc. Electrode 300 also includes one or more electrode leads 350 for communicating a treatment signal to exposed portion 310. Electrode lead 350 may be mechanically and electrically coupled to one or more cores of a wire 220 and, in some embodiments, to cores in the same wire 220 in which sensor lead 340 is coupled to. The treatment signal may be any suitable signal for excising tissue; for example, it may be a voltage, a current, etc.

Temperature sensor 330 and its lead(s) may be included in one, some, all, or none of plurality of elongated electrodes 210. Similarly, exposed portion 310 may be included in one, some, all, or none of elongated electrodes 210. In some embodiments, exposed portion 310 may function as a temperature sensor 330. In such a case, the electrode 210 may or may not include elements for delivering a treatment signal, and in such a case exposed portion 310 may or may not be sharpened to a point.

In one embodiment, electrode 300 includes multiple exposed areas. For example, insulated portion 320 may include one or more apertures for exposing portions of electrode 300. In one case, a portion of temperature sensor 330 may be exposed. In another case, a portion of one or more other element (e.g., a conductive material such as a metal, alloy, etc.) for delivering a treatment signal may be exposed. In such a case, electrode 300 may include multiple exposed portions for delivering multiple treatment signals either dependent or independent of one another. In yet another case, multiple exposed portions 310 may extend from one or more insulated portions 320, where each exposed portion 310 may or may not be sharpened to a point. In such a case, electrode 300 may also deliver multiple treatment signals, and may include none, one, or more temperature sensors 330.

In another embodiment, electrode 300 may be flexible or include one or more flexible elements. For example, electrode 300 may be a catheter, where leads are coupled to the catheter needle so as to communicate a treatment signal to the needle. The catheter may or may not include one or more temperature sensors.

In some embodiments, electrode 300 may be solid or include solid elements, such as a solid exposed portion 310 and temperature sensor 320. In other embodiments, electrode 300 may include hollow portions. For example, exposed portion 310 may include a hollow chamber. Other elements of electrode 300 may also include a hollow chamber. For example, a hollow chamber may extend the length of electrode 300. The hollow chamber may be operable to communicate fluid or the like. For example, blood, water, or other fluids may pass in either direction through the hollow chamber.

Electrode 300 in certain embodiments may include various components such as an exposed portion 310, an insulated portion 320, a temperature sensor 330, a temperature sensor lead 340, and electrode leads 350. However, it will be appreciated by those of ordinary skill in the art that the electrode could operate equally well by having fewer or a greater number of components than are illustrated in FIGS. 3A and 3B. Thus, the depiction of electrode 300 in FIGS. 3A and 3B should be taken as being illustrative in nature, and not limiting to the scope of the disclosure.

For example, in some embodiments electrode 300 may include a light source (not shown) such as a light emitting diode (LED). The light source may be operable to selectively output light such that a medical practitioner can visibly see the light. This may be useful for a practitioner to identify a particular, selected electrode. The light source may be provided in any suitable location, such as on an exterior surface of insulated portion 320 or beneath a transparent surface of insulated portion 320, or at an end of electrode 300 such as the end connected to flexible conductive wire 220. In one embodiment, computing device 120 provides an option via display device 130 for a user to locate or otherwise identify one or more electrodes. In response to receiving a user input selecting a particular electrode to locate, computing device 120 communicates an instruction to needle electrode assembly 170 and, in particular, to the electrode corresponding to the selected electrode. The instruction instructs the selected electrode to generate light such as via a light source provided in the electrode. Accordingly, in such embodiments, electrode 300 may include circuitry or other components operable to receive and interpret the received instruction and cause the light source to output light in response to receiving such an instruction.

Electrode Guide

A system will typically include an electrode guide or positioning device or apparatus. An electrode guide will typically be configured to engage electrodes of the system for assistance or facilitation of electrode positioning in the tissue of the patient. A guide may optionally include electrical connects that electrically couple with or in some manner facilitate, monitor, or affect energy delivery, monitoring, or control of current delivery. Various different designs or configurations of an electrode guide may be included in a system of the present invention.

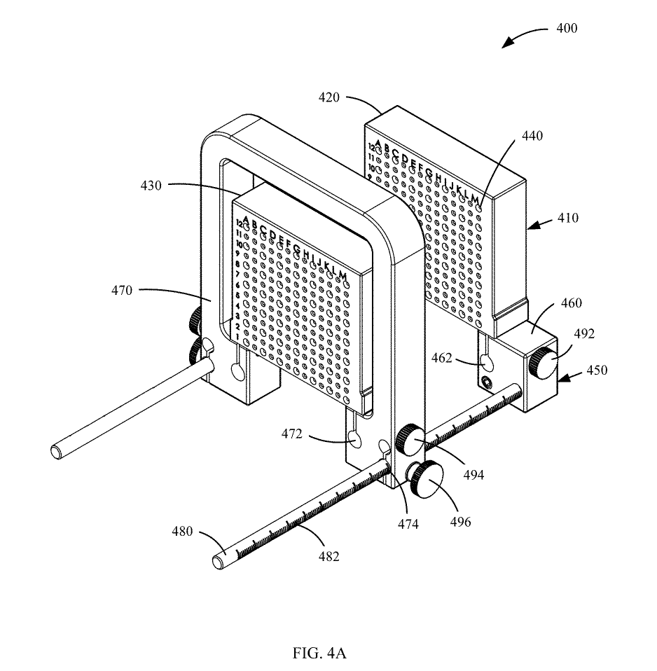

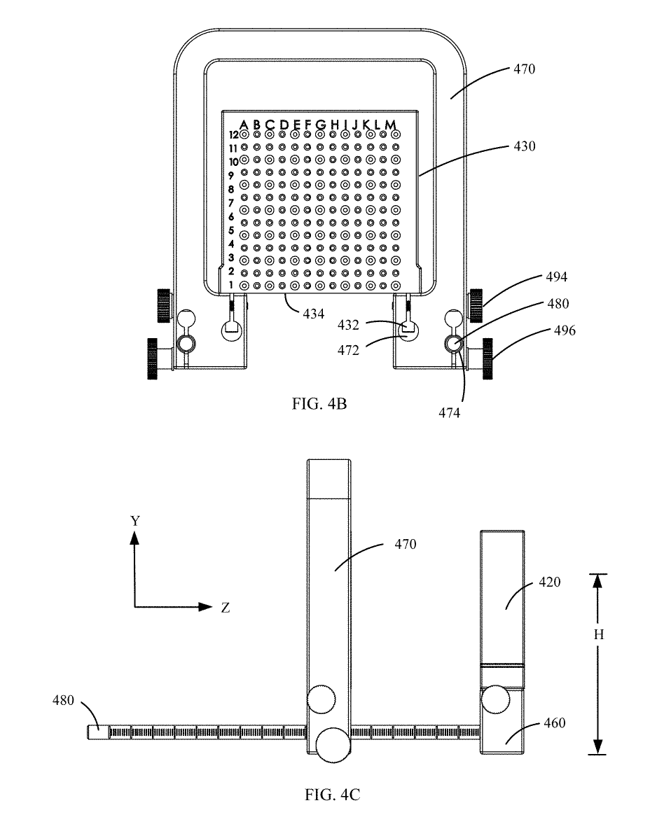

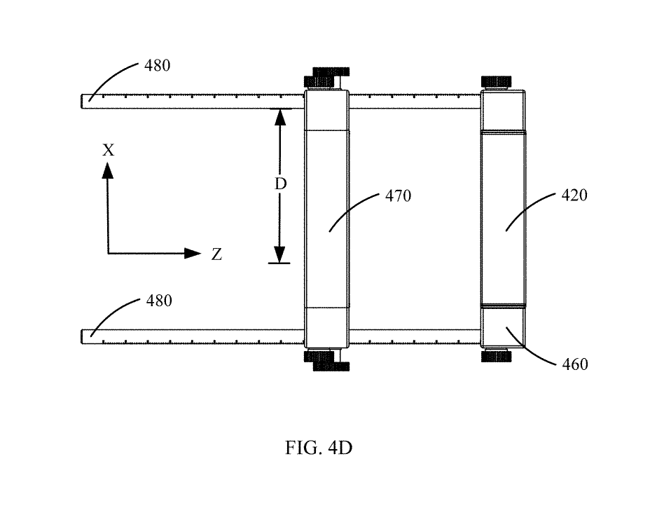

FIG. 4A is a profile view of an exemplary electrode guide 400 according to an embodiment. Electrode guide 400 includes a plurality of electrode templates and an adjustable template securing apparatus 450. In one embodiment, electrode guide 400 may correspond to the electrode guide 110 discussed with reference to system 100.

Electrode guide 400 may include any suitable number of templates. In one embodiment, electrode guide 400 includes a first electrode template 420 and a second electrode template 430. The electrode templates may be any suitable template operable to receive electrodes and, in some embodiments, allow the electrodes to pass therethrough. The electrode templates may be any suitable shape. For example, they may have a cross section that is square, rectangular, circular, oval, or any other suitable shape.

First electrode template 420 may include one or more apertures 440 formed partially or entirely through a depth of the template. Apertures 440 may have any suitable shape, such as circular, square, rectangular, oval, etc., and may be of any suitable size. For example, apertures 440 may be sized to receive an electrode such as elongated electrode 210 discussed with reference to FIG. 2A, and, in some embodiments, sized to form a friction fit with the electrode. In one embodiment, apertures 440 may be sized not to receive a portion of the electrode. For example, apertures 440 may be sized smaller than at least one dimension of enlarged portion 222 discussed with reference to FIG. 2A. In some embodiments, apertures 440 all have the same size, all have different sizes, or some have the same size while others have at least one different size. Apertures 440 may be spaced apart from one another by any suitable distance. For example, apertures 440 may be spaced apart from one another a distance of 1 mm, 2 mm, 3 mm, 4 mm, or 5 mm, or a distance in the range of 1 mm to 5 mm, or a distance of less than 1 mm or greater than 5 mm. Apertures 440 may also be arranged in any suitable pattern. For example, apertures 440 may be arranged in one or more squares, circles, ovals, rectangles, or the like, or a combination thereof. In one embodiment, apertures 440 are arranged in equally spaced rows and columns. Each row and/or column may have the same number or a different number of apertures 440. Second electrode template 430 may include one or more apertures similar to those discussed with reference to first electrode template 420.

In one embodiment, at least one of first electrode template 420 and second electrode template 430 includes an electronic circuit (not shown). For example, an electrode template may include a printed circuit board. The electronic circuit may include hardware and/or software for performing a variety of functions. For example, the electronic circuit may include conductive components for electrically coupling with an electrode disposed in an aperture of the electrode template. In such a fashion, a presence or absence of an electrode may be detected by the electronic circuit. The electronic circuit may then be communicatively coupled to other elements for communicating indications of the presence or absence of electrodes in one or more of the electrode templates. For example, the electronic circuit may be communicatively coupled to computing device 120.

In some embodiments, first electrode template 420 includes at least one securing element (not shown) extending from a surface of the template. For example, the at least one securing element may be a pin (not shown) extending from a bottom surface of the template. The securing element may be operable to mechanically couple first electrode template 420 to adjustable template securing apparatus 450. One embodiment of the at least one securing element is further discussed with reference to FIG. 4B. Second electrode template 430 may include one at least one securing element (not shown) similar to that discussed with reference to first electrode template 420. In one embodiment, first electrode template 420 and second electrode template 430 each include two or more securing elements.

Adjustable template securing apparatus 450 is operable to secure the plurality of electrode templates with respect to one another and adjust a distance between the plurality of electrode templates. In one embodiment, adjustable template securing apparatus 450 is operable to secure first electrode template 420 with respect to second electrode template 430 and adjust a distance between first electrode template 420 and second electrode template 430.

According to an embodiment, adjustable template securing apparatus 450 includes a first template mount 460, a second template mount 470, and a distance adjustment element 480. First template mount 460 may be operable to support first electrode template 420. For example, first template mount 460 may be mechanically couplable to distance adjustment element 480 and secure a position of first electrode template 420 relative to first template mount 460. First template mount 460 may be mechanically couplable to first electrode template 420 using any suitable mechanical coupling. For example, first electrode template 420 may be bonded to first template mount 460. For another example, first electrode template 420 may engage a cutout or aperture of first template mount 460. For yet another example, first template mount 460 may include one or more cutouts 462 each for receiving one or more securing elements (not shown) of first electrode template 420. In one embodiment, first template mount 460 includes two cutouts 462 arranged at opposite sides of first electrode template 420. In some embodiments, adjustable template securing apparatus 450 may also include at least one tightening element 492 for adjusting the strength of a mechanical coupling between first electrode template 420 and first template mount 460. For example, tightening element 492 may be a screw or other rotatable element operable to increase and/or decrease a size of cutout 462, where decreasing the size of cutout 462 results in an increased pressure on the securing element of first electrode template 420 by first template mount 460. In one embodiment, adjustable template securing apparatus 450 includes one tightening element 492 for each cutout 462.

Second template mount 470 may include some or all of the features discussed above for first template mount 460. For example, second template mount 470 may include a cutout 472 similar to cutout 462. Further, in some embodiments, adjustable template securing apparatus 450 may include one or more tightening elements 494 similar to the at least one tightening element 492, where the tightening elements 494 are operable to adjust the strength of a mechanical coupling between second electrode template 430 and second template mount 470.

In some embodiments, second template mount 470 is removably secured to distance adjustment element 480. Second template mount 470 may be removably secured to distance adjustment element 480 using any suitable mechanical coupling mechanism. For example, second template mount 470 may include a clasp, strap, or the like (not shown) for mechanically coupling to distance adjustment element 480. For another example, second template mount 470 may include one or more apertures 474 extending through a depth of second template mount 470. Aperture 474 may be any suitable shape and size to receive distance adjustment element 480 and allow distance adjustment element 480 to pass therethrough. In one embodiment, second template mount 470 includes two apertures 474 arranged on opposite sides of second electrode template 430. In some embodiments, template securing apparatus 450 may also include one or more tightening elements 496, similar to tightening element 492, for adjusting the strength of a mechanical coupling between distance adjustment element 480 and second template mount 470. For example, template securing apparatus 450 may include one tightening element 496 for each aperture 474.

Distance adjustment element 480 may be any suitable device operable to adjustably secure first electrode template 420 to second electrode template 430. In one embodiment, distance adjustment element 480 includes one or more cylindrically-shaped rods, although it may have any suitable cross-section shape, such as square, rectangular, oval, and the like. Distance adjustment element 480 may be removably secured to one or more of the plurality of electrode templates. In some embodiments, distance adjustment element 480 may be bonded to one or more of the plurality of electrode templates. For example, distance adjustment element 480 may be mechanically bonded to first template mount 460. In one embodiment, distance adjustment element 480 includes a pair of rods. Distance adjustment element 480 may include distance markers 482 that may be evenly spaced visual indicators indicating a distance along a length of distance adjustment element 480. For example, distance markers 482 may illustrate numerical values increasing in value from first template mount 460 so that a distance from first template mount 460 to second template mount 470 may be easily identified. Distance adjustment element 480 may be made of any suitable solid material, including metal, metal alloys, ceramic, polymers, etc.

FIG. 4B is a front view of the electrode guide of FIG. 4A. From the front view, the second electrode template 430 and second template mount 470 are visible, as are other elements such as apertures 474, adjustment elements 480, and tightening elements 494 and 496. Further, the previously mentioned securing elements 432 are shown.

Securing element 432 may be any suitable element extending from a surface of second electrode template 430 to removably secure second electrode template 430 to second template mount 470. For example, securing element 432 may be a pin-shaped extension extending from a bottom surface 434 of second electrode template 430, where a distal end of securing element 432 is sized larger than a proximal end of securing element 432 mechanically coupled to or formed with bottom surface 434. Securing element 432 may be sized to engage cutout 472. Further, tightening element 494 may be operable to increase or decrease a size of cutout 472 so as to increase or decrease a mechanical coupling between second electrode template 430 and second template mount 470.

FIG. 4C is a side view of the electrode guide of FIG. 4A. From the side view, first electrode template 420, first template mount 460, second template mount 470, and distance adjustment element 480, as well as various other components of electrode guide 400, are visible.

In one embodiment, first electrode template 420 and second electrode template 430 are each arranged such that corresponding apertures in the templates are provided at identical distances from distance adjustment element 480 along a Y-axis. For example, first electrode template 420 and second electrode template 430 may be arranged in parallel along a Z-axis and be oriented to extend along the Y-axis. Apertures 440 provided in first electrode template 420 may be aligned along the Y-axis with apertures 440 provided in second electrode template 430. For example, an aperture provided at location E-5 in first electrode template 420 may be provided at a same height (H) relative to distance adjustment element 480 as an aperture provided at location E-5 in second electrode template 430.

FIG. 4D is a top view of the electrode guide of FIG. 4A. From the top view, first electrode template 420, first template mount 460, second template mount 470, and distance adjustment element 480, as well as various other components of electrode guide 400, are visible.

In one embodiment, first electrode template 420 and second electrode template 430 are each arranged such that corresponding apertures in the templates are provided at identical distances from a distance adjustment element 480 along an X-axis. For example, first electrode template 420 and second electrode template 430 may be arranged in parallel along a Z-axis and be oriented to extend along the X-axis. Apertures 440 provided in first electrode template 420 may be aligned along the X-axis with apertures 440 provided in second electrode template 430. For example, an aperture provided at location E-5 in first electrode template 420 may be provided at a same distance (D) from a distance adjustment element 480 as an aperture provided at location E-5 in second electrode template 430.

By providing apertures 440 in first electrode template 420 in horizontal and vertical alignment with apertures 440 in second electrode template 430, the stability of an electrode passing through the templates may advantageously be increased as well as an accuracy of disposing the electrode into a target area.

Electrode guide 400 in certain embodiments is an apparatus for controlling the placement and positioning of electrodes and may include various components such as a plurality of electrode templates and an adjustable template securing apparatus. However, it will be appreciated by those of ordinary skill in the art that such an apparatus could operate equally well with fewer or a greater number of components than are illustrated in FIGS. 4A to 4D. Thus, the depiction of electrode guide 400 should be taken as being illustrative in nature, and not limiting to the scope of the disclosure.

For example, electrode guide 400 need not support electrodes operable to conduct current into a target area. Rather, in some embodiments, electrode guide 400 may be operable to support and/or guide radiation sources for applying radiation to a target area, such as in brachytherapy. In other embodiments, electrode guide 400 may be operable to support and/or guide needles or other devices for removing samples from the target area, such as in biopsies. Accordingly, electrode guide 400 may be operable to support a wide variety of instruments, medical or otherwise, for a variety of purposes.

Template

Electrode guides typically include one or more templates for operable to receive electrodes and, in some embodiments, allow the electrodes to pass therethrough. The templates may include one or more characteristics for resiliently positioning one or more electrodes received therein. By such resilient positioning, once an electrode has been placed in the template, accidental movement of the electrode may advantageously be reduced. As previously discussed, in one embodiment, the template(s) may include apertures suitably sized and shape to form a friction fit with an electrode. In other embodiments, the template(s) may include a friction plate operable to selectively change (e.g., increase or decrease) a friction force applied to one or more electrodes received by the template(s). By being operable to selectively change a friction force applied to electrodes, a position of electrodes may be substantially fixed once their appropriate position determined and, in some cases, re-positioning of the electrodes may easily be performed.

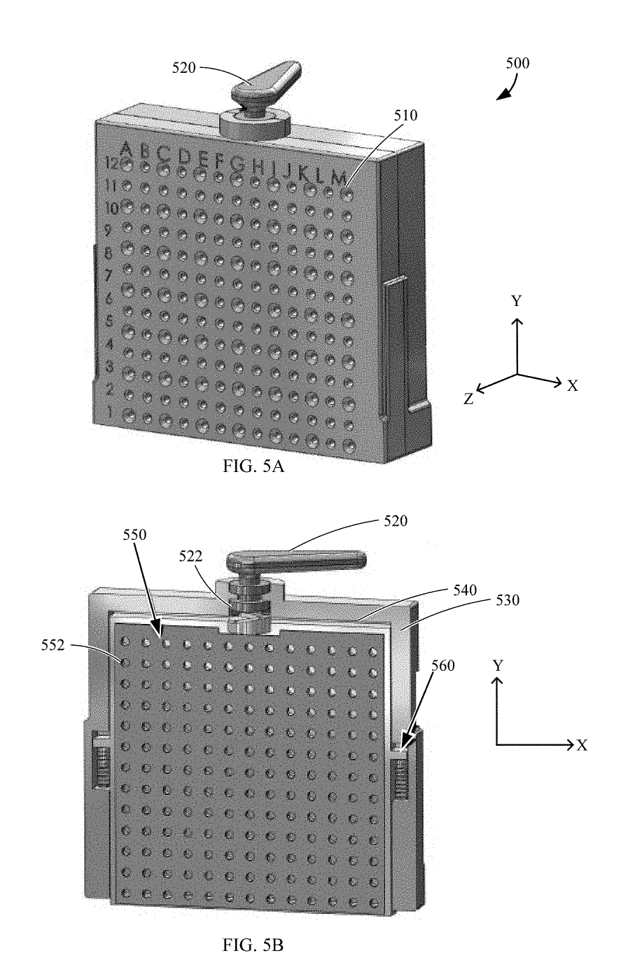

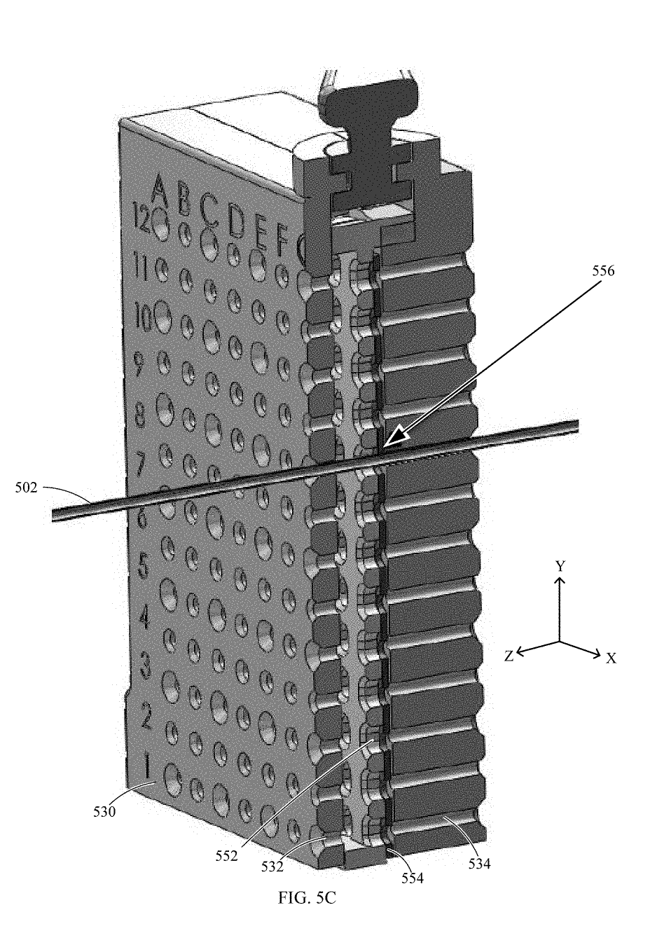

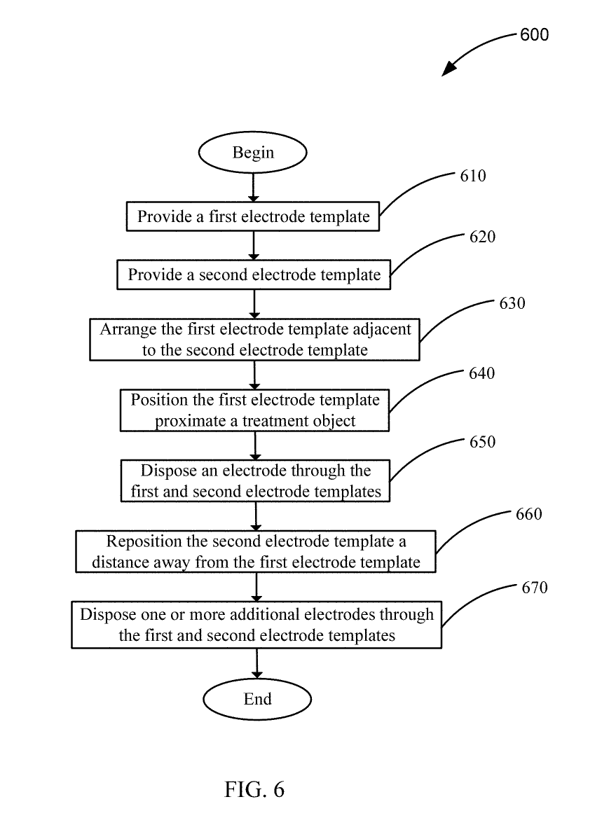

FIG. 5A is a profile view of a template 500 according to an embodiment. Template 500 may be a stand-alone template or, in some embodiments, may be the same as and incorporate one or more features of first electrode template 420 and/or second electrode template 430.

Template 500 includes one or more apertures 510 formed partially or entirely through a depth of the template. Apertures 510 may be similar to apertures 440. Template 500 also includes a friction adjustment mechanism 520 operable to change a friction force applied to one or more electrodes provided in apertures 510. Friction adjustment mechanism 520 may assume any suitable mechanical structure for causing displacement of elements of template 500. In one embodiment and as shown in FIG. 5A, friction adjustment mechanism 520 is a rotatable lever, where rotation in one direction causes an friction force applied to electrodes disposed in apertures 510 to increase, and rotation in an opposite direction causes the friction force applied to the electrodes to decrease. Other suitable mechanical structures include, but are not limited to, buttons, clamps, transverse actuators (i.e., non-rotational), etc.

FIG. 5B is a front view of the template of FIG. 5A. The front view is a view of template 500 in the X-Y plane, taken at a depth Z into template 500. From the front view, it is apparent that template 500 may include a housing 530 having a cavity 540 formed therein. Template 500 also includes a friction plate 550 disposed within cavity 540. Friction plate 550 is formed smaller than cavity 540 such that a position of friction plate 550 within cavity 540 may be changed in response to actuation of friction adjustment mechanism 520. Friction plate 550 includes one or more apertures 552 corresponding to apertures provided in frame 530 so as to form apertures 510 that pass through template 500. Apertures 552 may have any size and shape such that, upon actuation of friction adjustment mechanism 520, friction plate 550 operates to apply a friction force to electrodes disposed therethrough. For example, apertures 552 may be the same size, larger, or smaller than corresponding apertures in housing 530, and may all have the same or different sizes and/or shapes. In one embodiment, apertures 552 and the apertures in housing 530 are circular, and a diameter of apertures 552 is greater than or equal to the diameter of the apertures in housing 530. Friction plate 550 may be made of any suitable material for applying a friction force to electrodes disposed therethrough. For example, friction plate 550 may be made of a polymer such as plastic, one or more metals, ceramic, etc.

Template 500 may also include one or more elements operable to move friction plate 550 in conjunction with friction adjustment mechanism 520. For example, template 500 may include one or more return springs 560 operable to apply a return force to friction plate 550. In one embodiment, return springs 560 may apply a force on friction plate 550 in a direction opposite a force applied to friction plate 550 by friction adjustment mechanism 520. For example, friction adjustment mechanism 520 may include a cam 522 that, when rotated in a first direction, applies a linear force to friction plate 550 along the Y-axis. In response to applying the force to friction plate 550, friction plate 550 is caused to be displaced within cavity 540 along the Y-axis, such that a size of apertures 510 is effectively reduced. Return springs 560 apply a return force along the Y-axis in a direction opposite the direction of the force applied by rotation of cam 522 in the first direction. As a result, the force applied by return springs 560 operates to assist in returning friction plate 550 to its original position when cam 522 is rotated in a second direction opposite the first direction.

FIG. 5C is a cross sectional view of the template of FIG. 5A. From the cross sectional view, it is apparent that template housing 530 includes apertures 532 disposed on a front surface of housing 530 and apertures 534 disposed on a rear surface of housing 530. Apertures 532 and apertures 534 may have any suitable size and shape for receiving electrodes. In one embodiment, apertures 534 are elongated in a direction along the Z-axis, thereby increasing support for electrodes such as electrode 502 disposed therethrough.

According to some embodiments, friction plate 550 may be a multi-layered structure. A first layer 552 may be a support structure that is relatively hard. For example, first layer 552 may be made of metal, ceramic, or one or more other relatively hard materials. A second layer 554 may be supported by first layer 552 and, in some embodiments, may be formed on a surface of first layer 552. Second layer 554 may be made of a relatively soft material (compared to first layer 552). For example, second layer 554 may be made of a polymer such as plastic. In these embodiments, first layer 552 may mechanically interact with cam and return springs 560 and, by its relatively hard physical nature, be resilient to long-term use and the wear resulting therefrom. Second layer 554, on the other hand, may mechanically interact with one or more electrodes 502 passing through friction plate 550 and, by its relatively soft physical nature, be operable to apply a friction force to electrode 502 without damaging electrode 502. To facilitate such an operation, first layer 552 and second layer 554 may each have apertures corresponding to apertures of housing 530, where apertures of second layer 554 may be smaller than apertures of first layer 552. As a result, second layer 554 may include an electrode interference portion 556 operable to engage or otherwise mechanically interfere with electrode 502.

Template 500 in certain embodiments is a device operable to selectively apply a friction force to received electrodes and may include various components such as a movable friction plate, friction adjustment mechanism, and return springs. However, it will be appreciated by those of ordinary skill in the art that such a system could operate equally well with fewer or a greater number of components than are illustrated in FIGS. 5A to 5C. Thus, the depiction of template 500 should be taken as being illustrative in nature, and not limiting to the scope of the disclosure.

FIG. 6 is a flowchart depicting example operations of a method 600 for controlling a position of one or more elongated electrodes. The electrodes may be any suitable elongated element, including any of the electrodes previously discussed such as electrode 102 of FIG. 1A.

In operation 610, a first electrode template is provided. The first electrode template may be any suitable device for receiving and supporting elongated electrodes. For example, the first electrode template may correspond to first electrode template 420 discussed with reference to FIG. 4A. Accordingly, the first electrode template may include a plurality of apertures arranged to receive one or more elongated electrodes.

In operation 620, a second electrode template is provided. The second electrode template may be any suitable device for receiving and supporting elongated electrodes. For example, the second electrode template may correspond to second electrode template 430 discussed with reference to FIG. 4A. Accordingly, the second electrode template may include a plurality of apertures arranged to receive one or more elongated electrodes.

In operation 630, the first electrode template is arranged a first distance from the second electrode template. For example, second electrode template 430 may be arranged to contact first electrode template 420 such that the first distance is 0 mm. For another example, second electrode template 430 may be arranged a distance of 10 mm, 20 mm, or 30 mm from first electrode template 420, or in a range from 10 mm to 30 mm, or less than 10 mm or greater than 30 mm.

The first electrode template may be arranged a first distance from the second electrode template using any suitable movement and securing mechanisms. For example, with reference to FIG. 4A, a position of first electrode template 420 may be secured relative to a position of distance adjustment element 480 using first template mount 460. For example, first electrode template 420 may be removably secured to first template mount 460 by engaging at least one tightening element 492. A position of second electrode template 420 may similarly be secured relative to a position of distance adjustment element 480 using second template mount 470. For example, second electrode template 430 may be removably secured to second template mount 470 by engaging at least one tightening element 494. Second electrode template 430 may be positioned proximate to first electrode template 420 by passing distance adjustment elements 480 through apertures 474 of second template mount 470. Second electrode template 430 may then be positioned along distance adjustment element 480 a first distance from first template mount 460. Once second template mount 470 has been arranged the first distance from first template mount 460, a position of second template mount 470 relative to first template mount 460 may be secured by engaging tightening elements 496. In some embodiments, the positioning of the second electrode template may be mechanically and electronically controlled by any suitable control apparatus such as computing device 120 discussed with reference to FIG. 1B.

In operation 640, the first electrode template is positioned proximate a treatment object. The treatment object may be any suitable object for which penetration of one or more electrodes is desired. For example, the treatment object may be a patient for which tissue ablation of a prostate (P) such as that discussed with reference to FIG. 1A is desired. By proximate positioning, the first electrode template is arranged at a fixed distance from the treatment object. For example, first electrode template 420 may be arranged to contact a surface of the treatment object. For another example, first electrode template 420 may be arranged a distance of 10 mm, 20 mm, or 30 mm from a surface of the treatment object, or in a range from 10 mm to 30 mm, or less than 10 mm or greater than 30 mm. In some embodiments, the positioning of the first electrode template may be mechanically and electronically controlled by any suitable control apparatus such as computing device 120 discussed with reference to FIG. 1B.

In operation 650, an electrode is disposed through the first and second templates. For example, an elongated electrode may be disposed through an aperture of second electrode template 430 (e.g., an aperture located at template position E-5) and through a corresponding aperture of first electrode template 420 (e.g., an aperture located at template position E-5). The electrode may be disposed to first enter and pass through the second template and then enter and pass through the second template. Upon passing through the second template, the electrode may penetrate a surface of the treatment object.

In one embodiment, the electrode may penetrate the treatment object to a maximum desired depth. For example, with reference to FIG. 1A, a maximum desired depth may correspond to a rear wall of the prostate (P) that is located opposite an electrode-penetrating surface of the patient. In some embodiments, the treatment object may be monitored to determine the maximum desired depth. For example, imaging device/system 112 may graphically monitor a location of the electrode within the treatment object. The monitored images of the electrode and treatment object may be communicated for display by, for example, display device 130. In one embodiment, computing device 120 may determine the maximum desired depth by setting the depth based on a minimum distance between a penetration end of the electrode and the rear wall of the prostate (P). Further, in some embodiments, computing device 120 may be operable to control the penetration of electrode into the treatment object.

In operation 660, the second electrode template is repositioned to be a second distance from the first electrode template, where the second distance is greater than the first distance. For example, tightening elements 496 may be relaxed so as to enable second template mount 470 to move along distance adjustment element 480. Second template mount 470 may then be moved in a direction away from first template mount 460, so as to increase a distance between first electrode template 420 and second electrode template 430. This may be performed while maintaining a fixed distance between first electrode template 420 and the treatment object. In one embodiment, the elongated electrode includes an enlarged portion such as enlarged portion 222 discussed with reference to FIG. 2A, where the enlarged portion is sized to mismatch the aperture of second electrode template 430 which the electrode is disposed in. Second template mount 470 may then be moved away from first template mount 460 until the aperture which the electrode is disposed in contacts the enlarged portion of the electrode. In some embodiments, second template mount 470 may then be re-secured to distance adjustment element 480 by, for example, re-engaging tightening elements 496.

By first disposing an electrode to a maximum depth and then arranging the second electrode template to be a distance from the first electrode template based on the maximum depth of the electrode, the maximum penetration depth of one or more additional electrodes may advantageously be determined and fixed.

In operation 670, one or more additional electrodes may be disposed through the first and second electrode templates. For example, one or more additional electrodes may be disposed in apertures surrounding the aperture in which the elongated electrode was disposed, and may be disposed while maintaining a position of first electrode template 420 relative to second electrode template 430. In some embodiments, the one or more additional electrodes may have the same size and shape of the previously disposed electrode, and in some cases, may have a similar enlarged portion such as that discussed with respect to the previously disposed electrode. By having the same enlarged portion and securing the second electrode template at the second distance, the one or more additional electrodes are prevented from exceeding the maximum depth.

In some embodiments, the first distance and/or second distance and thus, in some cases, the maximum depth, may be recorded using, for example, distance markers 482. The measurements may be stored in any suitable storage medium such as storage device 124. In some embodiments, multiple maximum depths can be determined and stored for different electrodes. For example, with reference to FIG. 1A, the rear surface of prostate (P) is contoured. For example, prostate (P) may be substantially round. Accordingly, a maximum depth may differ based on a location along the rear surface of prostate (P), and thus a maximum depth of the electrodes may differ based on a location of the electrodes in the guide template. In one embodiment, the maximum depth for one or more additional electrodes may be determined by performing operations 550 and 560 for an additional electrode, and may include at least partly retracting from the guide template any previously disposed electrodes.

It should be appreciated that the specific operations illustrated in FIG. 6 provide a particular method of controlling a position of one or more elongated electrodes, according to certain embodiments of the present invention. Other sequences of operations may also be performed according to alternative embodiments. For example, alternative embodiments of the present invention may perform the operations outlined above in a different order. Moreover, the individual operations illustrated in FIG. 6 may include multiple sub-operations that may be performed in various sequences as appropriate to the individual operation. Furthermore, additional operations may be added or existing operations removed depending on the particular applications. One of ordinary skill in the art would recognize and appreciate many variations, modifications, and alternatives.

Electrode Control Software

The system further includes software or computer executable instructions that, when executed, cause the system to perform one or more actions or steps of the energy delivery as described herein. The software may provide a user interface for a user to control operation of the electrodes, and may be operable to control various elements of the system in accordance with the user inputs and/or receive and communicate to the user information from the electrodes such as temperature readings. The user interface may include visual depictions of electrodes that are to be controlled, and may also include any suitable input mechanism for receiving a user selection of control parameters. The control parameters may include, for example, an indication of specific electrodes for which a voltage is to be applied, a duration of time for which the voltage is to be applied to the chosen electrodes, and a desired temperature at which the controlled electrodes are to achieve.

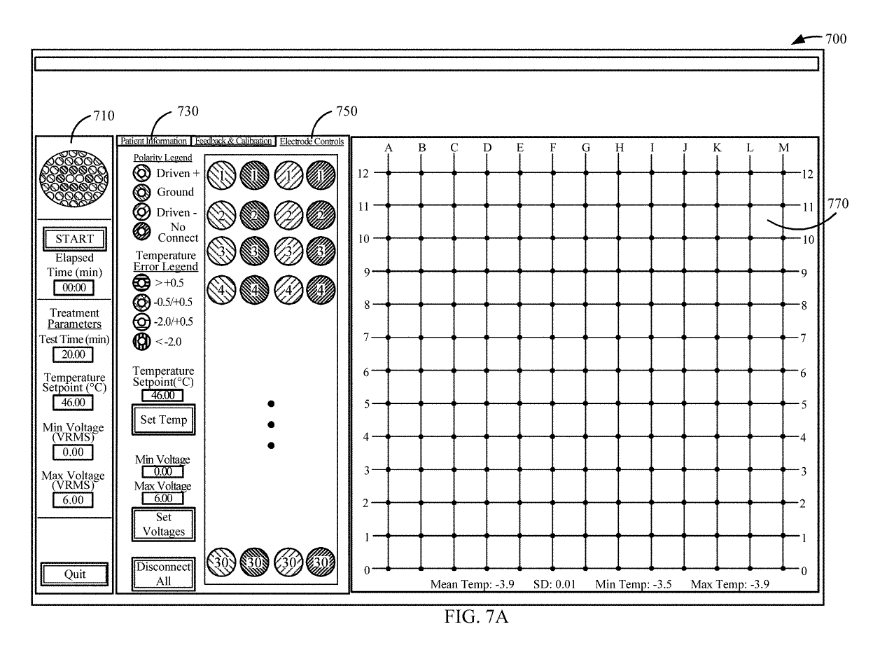

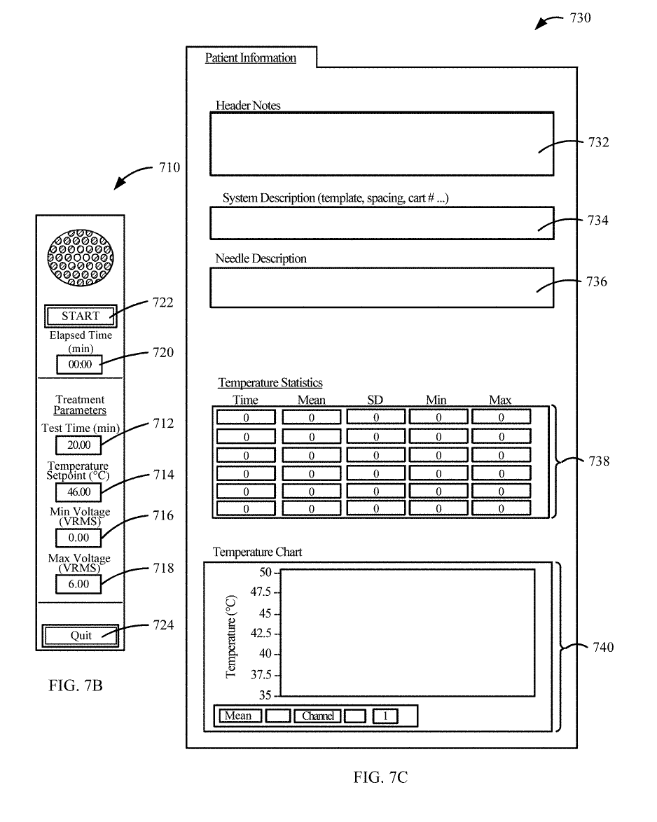

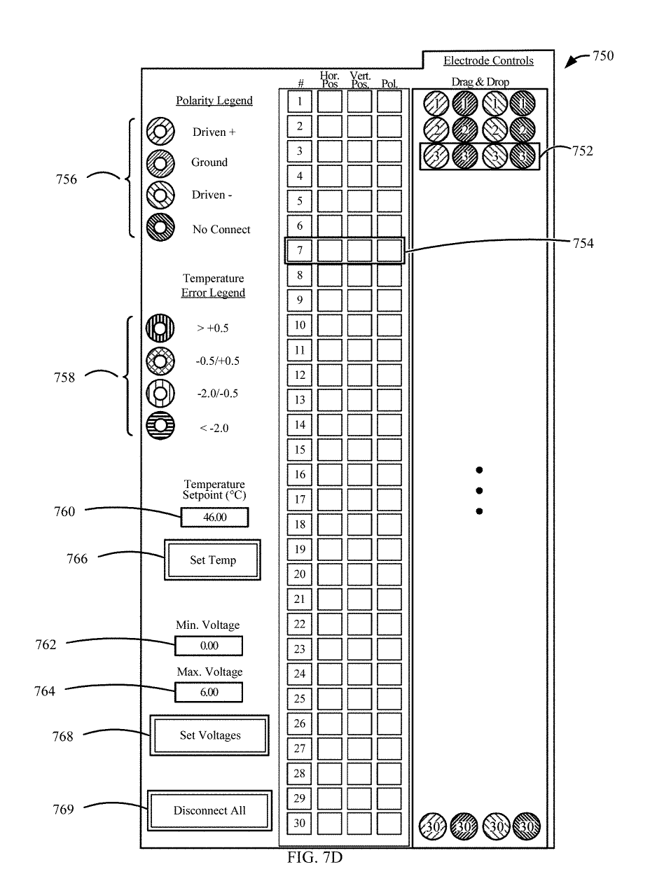

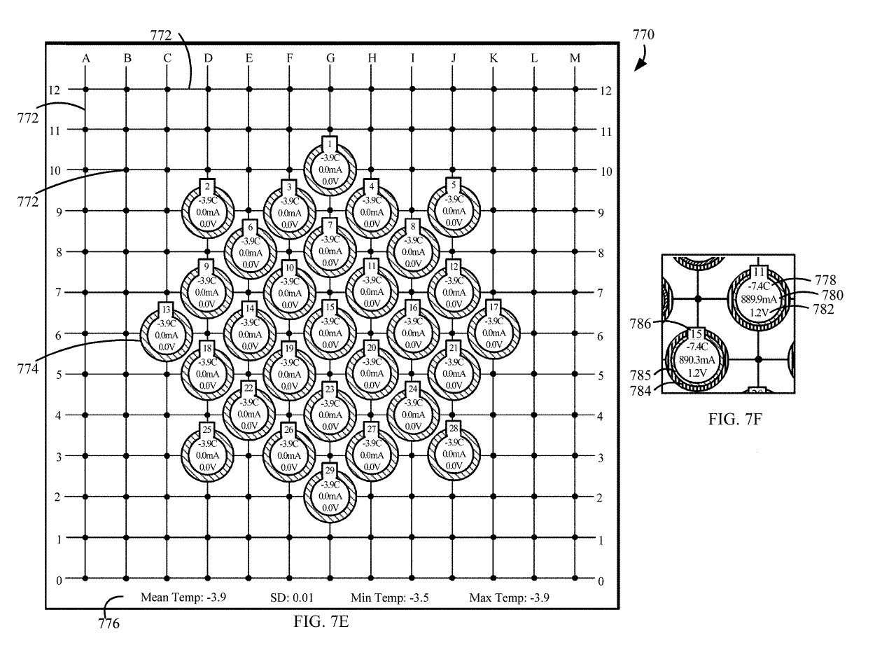

FIG. 7A shows a user interface 700 for monitoring and controlling a plurality of electrodes according to an embodiment. According to one embodiment, user interface 700 is generated and controlled by computing device 120, and displayed on display device 130. An operator may feed input into user interface 700 in a variety of ways. For example, an operator may utilize any of the input devices previously discussed, such as mice, keyboards, trackballs, touch-screens, etc.

Computing device 120 may have stored therein computer software for rendering user interface 700 based on various inputs, such as inputs from amplifier board 140. The computer software may be further functional to receive user inputs via one or more of the previously discussed input devices, and communicate corresponding control information to, e.g., amplifier board 140, for controlling electrodes 210 so as to heat a target area or volume, such as prostate tissue (P) (FIG. 1A) to a selected temperature or temperature range. In one embodiment, the computer software may be stored on storage device 124 and executed by processor 122.

User interface 700 may include one or more elements in one or more frames, windows, stacked tabs, or screens for display on one or more display devices. The elements may display various information pertaining to electrodes controlled by system control unit 108, such as voltages and currents applied to electrodes, temperature readings measured by electrodes, and the like. In some embodiments, the elements may also display various information pertaining to controlling the electrodes, such as control or treatment parameters.

According to one embodiment, user interface 700 includes a treatment parameter element 710, a patient information element 730, an electrode control element 750, and an electrode status element 770. At least some of the elements may be arranged proximate to one another. For example, electrode control element 750 may be arranged adjacent to electrode status element 770, and electrode control element 750 may include electrode activation elements that may be dragged from electrode control element 750 and dropped onto locations of electrode status element 770 to cause electrodes to be selectively activated.

FIG. 7B shows a treatment parameter element 710 of the user interface 700 of FIG. 7A. Treatment parameter element 710 may include one or more treatment parameter values, such as a test time 712, a desired electrode temperature 714, a minimum electrode voltage 716, and a maximum electrode voltage 718. Test time 712 may illustrate a total time in which system control unit 108 causes power to be applied to the electrodes of electrode assembly 170. Desired electrode temperature 714 may illustrate a maximum temperature allowable for any of the electrodes in electrode array 170, as measured by thermistors within or in proximity to the electrodes. The maximum temperature may correspond to a selected or desired temperature of a target area, where a temperature range may be defined by the selected temperature plus an acceptable deviation from the selected temperature. In some cases, the acceptable deviation may be a characteristic of the treatment system, and in other cases, the acceptable deviation may be input or selected e.g. by a treatment planning, a user, or practitioner. Additionally or alternatively, a user may enter a selected parameter or set of parameters such as voltage or a range of voltages to apply to the electrodes of electrode assembly 170, where the selected parameter or set of parameters may be recognized or processed to identify a corresponding target temperature or temperature range. For example, instead of the practitioner entering a selected temperature, the practitioner may enter a selected voltage or range of voltages, where a correspondence between temperature and voltage may be recognized. Accordingly, minimum electrode voltage 716 and maximum electrode voltage 718 may respectively illustrate minimum and maximum voltages allowable for any of electrodes in electrode array 170. According to some embodiments, the treatment parameter values may be input into corresponding fields by an operator via an input device. According to other embodiments, the treatment parameter values may be predetermined and pre-stored in, for example, storage device 124.

Treatment parameter element 710 may include features in addition or alternatively to the aforementioned treatment parameter values. For example, treatment parameter element 710 may include an elapsed time value 720 that shows an amount of time that has elapsed since a particular treatment had begun (i.e., since electrodes in electrode array 170 were initially activated in a given session). For another example, treatment parameter element 710 may include a start button 722 and a quit button 724, activation of start button 722 causing treatment to begin, and activation of quit button 724 causing user interface 700 to terminate.

FIG. 7C shows a patient information element 730 of the user interface 700 of FIG. 7A. Patient information element 730 may include various information pertaining to a particular patient and the equipment used for that particular patient, including current information and historical information. For example, patient information 730 may include: a header notes field 732 for allowing the operator to enter general comments regarding a patient, setup, or treatment plan; a system description field 734 for allowing the operator to enter information pertaining to the system setup, such as template size, support equipment information, any non standard equipment adjustments or configurations; and a needle description field 736 for allowing the operator to enter information describing the electrodes used. Information input into these fields may be stored in a unique file associated with a particular patient in, for example, storage device 124, and subsequently displayed in, for example, patient information element 730. In some embodiments, such information is already stored in a device such as storage device 124 and subsequently displayed in patient information element 730.

Patient information element 730 may include features in addition or alternatively to the aforementioned information. For example, patient information element 730 may include temperature statistics 738 that illustrates various statistics concerning the temperature of one or more electrodes in electrode assembly 170. Such statistics may include a time indicator indicating a time that particular temperature statistics are applicable, a mean temperature of the electrode(s), a standard deviation of the temperature of the electrode(s), a minimum temperature of the electrode(s), and/or a maximum temperature of the electrode(s). Such information may be calculated by, for example, processor 122, based on temperature measurements received from amplifier board 140 and may, advantageously, be reviewed by an operator during a test to ensure that a treatment is trending as expected.

Patient information element 730 may additionally or alternatively include a temperature chart 740, where the temperature chart 740 may also illustrate various temperature statistics. For example, temperature chart 740 may graphically illustrate temperature statistics such as mean temperature, standard deviation, etc., with respect to time. The time duration may be predetermined or user selectable, and may include a time range from the beginning of treatment to a current time, or a subset of such a time range. Such information may be calculated by, for example, processor 122 based on temperature measurements received from amplifier board 140 and may, advantageously, be reviewed by an operator during a test to ensure stability of the treatment.

FIG. 7D shows an electrode control element 750 of the user interface 700 of FIG. 7A. Electrode control element 750 may, in some embodiments, be used to configure the output delivered to the electrodes and select the electrodes to be involved in a particular treatment.

Electrode control element 750 includes, for each electrode, an electrode polarity selector 752. In this embodiment, electrode control element 750 is operable to control 30 electrodes numbered 1 to 30, although any number of electrodes may be controlled. Electrode polarity selector 752 may include a graphical representation of a particular electrode (e.g., electrode number 3) for a number of different polarities of the electrode. For example, polarity selector 752 may include a graphical representation for applying a positive voltage to the electrode (i.e., 0 degree phase), a graphical representation for grounding the electrode, a graphical representation for applying a negative voltage to the electrode (i.e., 180 degree phase), and a graphical representation for electrically disconnecting the electrode (i.e., high impedance). Each graphical representation may have a unique color. In this embodiment, an operator may ‘drag and drop’ a graphical representation of a particular electrode driven at a particular polarity to a location on electrode status element 770. Doing so may cause amplifier board 140 to generate a voltage of the particular polarity and apply the voltage to an electrode of electrode assembly 170 corresponding to the location on electrode status element 770. In such a fashion, electrode patterns may advantageously be created quickly and easily.

According to some embodiments, polarity selector 752 may only include a graphical representation for applying a connected or disconnected electrode. For example, when control unit 108 is used for treating a patient, the operator may only need to select connected electrodes to be involved in a treatment and a high impedance needle to be used as temperature sensors or for other purposes. According to other embodiments, polarity selector 752 may include all of the aforementioned graphical representations. For example, when control unit 108 is used for system testing and/or research and development.

Electrode control element 750 may also include location and polarity information 754 concerning the electrodes. For example, electrode control element 750 may include, for each electrode, a horizontal position, a vertical position, and a polarization. In one embodiment, computing device 120 may calculate and cause such values to be displayed based on the operator's selection of locations on electrode status element 770. In other embodiment, such elements may be fields in which a user may enter the horizontal position, vertical position, etc., rather than performing the aforementioned drag-and-drop technique.

Electrode control element 750 may include various legends for aiding an operator in understanding the electrode control element 750. For example, electrode control element 750 may include a polarity legend 756 and/or a temperature error legend 758. Polarity legend 756 may include information indicating a correspondence between colors of graphical representations of polarity selector 752 and polarities applied to electrodes. Temperature error legend 758 may include information indicating a correspondence between colors of electrode status element 770 and a difference between a current electrode temperature and a desired electrode temperature.

Electrode control element 750 may also include one or more treatment parameters, in addition or alternative to those previously discussed with reference to FIG. 7B. For example, electrode control element 750 may include a desired electrode temperature 760, a minimum electrode voltage 762, and a maximum electrode voltage 764. An operator may enter values into these fields, or, in some embodiments, these fields may be automatically populated if the operator enters data into the corresponding fields in treatment parameter element 710.

Electrode control element 750 may also include one or more buttons, activation of which may cause computing device 120 to perform select functionality. For example, electrode control element 750 may include: a set temperature button 766, activation of which may cause computing device 120 to record and store the value entered in desired electrode temperature field 760 for a subsequent treatment; a set voltage button 768, activation of which may cause computing device 120 to record and store the value entered in minimum electrode voltage field 762 and maximum electrode voltage field 762 for a subsequent treatment; and a disconnect all button 769, activation of which may cause all of the controlled electrodes to be electrically disconnected.

FIG. 7E shows an electrode status element 770 of the user interface 700 of FIG. 7A. Electrode status element 770 includes a grid array 772 comprising a plurality of horizontal lines, vertical lines, and intersection points. The grid array 772 may be a graphical representation of a device for positioning electrodes of electrode assembly 170, e.g., a graphical representation of electrode guide 110. Intersection points may correspond to particular locations where electrodes in electrode guide 110 may be positioned, and may include horizontal position references (e.g., letters A to M) and vertical position references (e.g., numbers 0 to 12).

Electrode status element 770 also includes an electrode representation 774 which is a graphical representation of an electrode in electrode assembly 170. Any number of electrode representations 774 may be provided for a corresponding number of electrodes in electrode assembly 170. The number of electrode representations may be the same or different than the number of electrodes in electrode assembly 170. For example, some electrodes in electrode assembly 170 may not be used or positioned in electrode guide 110, thus obviating the need for a graphical representation or control mechanism. Further, the electrode representations 774, and corresponding electrodes in electrode assembly 170, may be provided in any suitable arrangement. For example, the electrodes and their graphic representations may be provided in square, circular, oval, or other arrangement. In some embodiments, the electrodes and electrode representations are provided in arrangements suitable for confined tissue ablations.

Electrode status element 770 may also include summary statistics information 776 for providing a summary of information illustrated by electrode representations 774. For example, summary statistics information 776 may include one or more of a mean temperature of all electrodes, a standard temperature deviation of all electrodes, a minimum electrode temperature, and a maximum electrode temperature.

FIG. 7F shows a magnified portion of an electrode status element as FIG. 7E. As shown in this embodiment, each electrode representation 774 may provide various information concerning the corresponding electrode. For example, electrode representation 774 may include one or more of: a current temperature 778 of the electrode, a current electrical current 780 of the electrode, and a current electrical voltage 782 of the electrode. Electrode representation 774 may also include relative information as well. For example, electrode representation 774 may include a relative temperature indicator 784 indicating a difference between a current temperature of the electrode and a desired temperature of the electrode. The relative temperature indicator 784 may be color coded. For example, with reference to temperature error legend 758, a color of relative temperature indicator 784 may illustrate that a current temperature of the electrode is greater than the desired temperature (e.g., is greater than 0.5 degrees above the desired temperature), is in a range around the desired temperature (e.g., is greater than 0.5 degrees below the desired temperature and less than 0.5 degrees above the desired temperature), is in a range below the desired temperature (e.g., is less than 0.5 degrees below the desired temperature and greater than 2 degrees below the desired temperature), or is significantly below the desired temperature (e.g., is more than 2 degrees below the desired temperature). Electrode representation 774 may also include a polarity indicator 785 indicating a polarity of the voltage of the electrode. The polarity indicator 785 may be color coded. For example, a color of polarity indicator 785 may illustrate that a current polarity of the electrode is positive, or the color may illustrate that the current polarity of the electrode is negative. In some embodiments, each electrode representation 774 may also include an electrode identifier 786 uniquely identifying the particular electrode.

User interface 700 in certain embodiments is an interface for monitoring and controlling a plurality of electrodes, and may include various components such as a treatment parameter element 710, a patient information element 730, an electrode control element 750, and an electrode status element 770. However, it will be appreciated by those of ordinary skill in the art that the user interface could operate equally well by having fewer or a greater number of components than are illustrated in FIGS. 7A to 7F. Thus, the depiction of user interface 700 in FIGS. 7A to 7F should be taken as being illustrative in nature, and not limiting to the scope of the disclosure.

Electrode Control Algorithm

The system may execute an electrode control algorithm in which voltages are applied to the electrodes in accordance with the control algorithm. The electrode control algorithm may be implemented in hardware using any suitable electronic components. For example, the algorithm may be programmed into one or more EPROM's, EEPROM's, SRAM, or other programmable logic. Some or all of the electrode control algorithm may also or alternatively be implemented in software executable by any suitable computer processor. For example, the algorithm may be programmed in Fortran, Pascal, C, C++, Visual Basic, or any other suitable programming language.

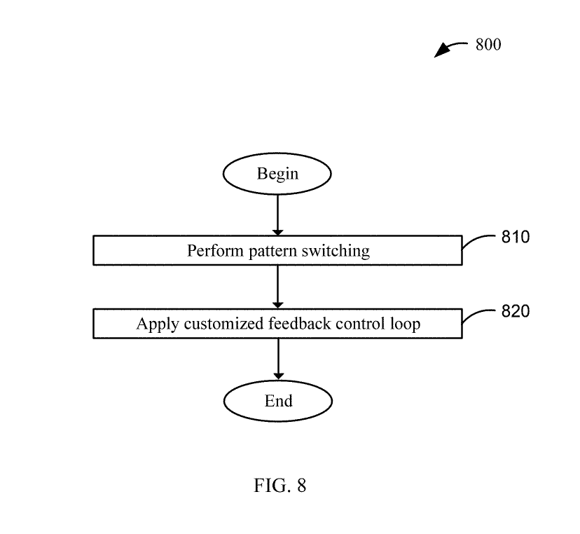

FIG. 8 is a flowchart depicting example operations of a method 800 for controlling electric fields created by a plurality of electrodes according to an embodiment. In one embodiment, electrodes in electrode assembly 170 (discussed with reference to FIG. 1B) may be controlled to deliver maximum electric fields to a tissue of patient while maintaining a temperature below a thermal limit at each electrode. Method 800 may, in some embodiments, advantageously compensate for one or more variables in a tissue ablation, such as: variations in tissue impedance; variations in needle spacing due to needle drift or bending; variations in needle insertion depth due to curving body geometry; non-uniform heat loss due to blood circulation, tissue type or multiple tissue types, depth within the body, patient body temperature, and/or needles at an edge of a treatment pattern rather than the center; non-symmetrical needle patterns due to odd-shaped treatment areas; and lag time between tissue heating and being measured by thermistors.

Method 800 comprises two operations. In operation 810, computing device 120 (discussed with reference to FIG. 1B) performs pattern switching. That is, after treatment begins, voltages may be provided to electrodes in electrode assembly 170 to create differences in electric potentials between some adjacent electrodes. As a result, current will tend to flow through the medium in which the electrodes are located (e.g., tissue) between electrodes having an electric potential difference, so as to create a current flow pattern for the electrodes. The voltage provided to the electrodes may then be changed so as to create electric potential differences between some other adjacent electrodes, so as to create a different current flow pattern for the electrodes. While a current flow pattern refers to the pattern of currents flowing between electrodes, an electric voltage pattern refers to the pattern of electric voltages applied to the electrodes so as to generate a current flow pattern.

Computing device 120 may switch between any suitable number of electric voltage patterns. For example, computing device 120 may switch between two, three, four, or greater than four unique electric voltage patterns. Computing device 120 may switch between electric voltage patterns at any suitable rate, such as once every second, once every two seconds, once every three seconds, or once for every time period in a range between one second and three seconds, or once for every time period greater than three seconds, or once for every time period less than one second. Further, computing device 120 may repetitively switch between sequences of electric voltage patterns for any suitable treatment period, such as 20 minutes, 40 minutes, 60 minutes, or in a range between 20 minutes and 60 minutes, or less than 20 minutes, or greater than 60 minutes.

Numerous advantages may arise out of performing pattern switching. For example, if a first voltage is applied to a number of first electrodes and a second voltage is applied to a greater number of second electrodes in a given pattern, the first electrodes will have a higher current density and thus higher temperature than the second electrodes due to their lower numbers. By switching from the given pattern to a different pattern, a balance of voltages may be altered (e.g., the first voltage may be applied to smaller number of electrodes than the second voltage), and current densities and thus electrode temperatures may be averaged out over all of the electrodes. Averaging the electrode temperatures over all of the electrodes may advantageously reduce the number and/or effect of localized hot spots.

For another example, a single electric voltage pattern cannot evenly address all of the corner or outlying electrodes simultaneously. That is, while one outer electrode may have an electric potential difference with three or four adjacent electrodes, thereby creating a high current density for that outer electrode, another outer electrode may have an electric potential difference with only one adjacent electrode, thereby creating a relatively low current density for that outer electrode. By switching between voltage patterns, the number of electric potential differences between an outer electrode and adjacent electrodes may change, thereby averaging the current density and temperature of the outer electrodes over the treatment period.

In operation 820, computing device 120 applies a customized feedback control loop to control the electrical voltage provided to the electrodes. The feedback control loop may incorporate any suitable feedback control, including one or more of closed-loop feedback and open-loop feedback, and including one or more of proportional control, proportional-integral control, proportional-integral-derivative control, bistable control, and hysteretic control.

The customized feedback control loop may control the electrical voltage provided to the electrodes based on any suitable inputs and/or measured signals. In one embodiment, the electrical voltage of an electrode may be controlled based on a temperature difference which is set based on an adjacent electrode. For example, the temperature difference may be the difference between a temperature of an electrode for which the electrical voltage to be applied is being determined and a temperature of an adjacent electrode. For another example, the temperature difference may be the difference between a temperature of the adjacent electrode and a desired temperature. Using a temperature difference based on a temperature of an adjacent electrode may advantageously prevent and/or reduce the likelihood of overheating the adjacent electrode.

In one embodiment, the electrical voltage of an electrode may be controlled using an estimate of the voltage provided at the electrode. For example, computing device 120 may calculate a feedback control error based on the difference between an electrode temperature and another temperature (such as a desired temperature or a temperature of an adjacent electrode). The electrical voltage of the electrode may then be determined based on the calculated feedback control error. In determining the electrical voltage to be applied to the electrode, instead of using an electrical voltage of the electrode, computing device 120 may use an estimated voltage at the electrode, the estimated voltage being an estimate of voltages provided by other electrodes at the electrode under consideration. The estimated voltage provided by other electrodes may be determined by summing the voltages of each of the other electrodes adjusted by a distance of the other electrodes from the electrode, and averaging the result based on the number of other electrodes. By using an estimated voltage provided at an electrode by other electrodes rather than using the voltage of the electrode itself, a current flow between the electrode and other electrodes may be more accurately controlled, thereby increasing the accuracy of heat generation.

In some embodiments, computing device 120 may perform a proportional, proportional-integral, or proportional-integral-derivative control process, where input mechanisms may be controlled using, for example, a weighted sum of errors, integration errors, and derivative errors. In one embodiment, the input mechanisms may be voltages applied to each of the electrodes, and the errors may be a difference between an actual electrode temperature and a desired electrode temperature. Accordingly, via a proportional-integral control process, computing device 120 may track each electrode temperature and adjust each electrode voltage to deliver as much energy as possible without exceeding a thermal limit.

In one embodiment, the customized feedback control loop includes, for each electrode, measuring a current temperature of the electrode. For example, this may be performed using temperature measurements from a thermistor arranged within or proximate to the electrode. The control process may further include, for each electrode, calculating a difference between a current temperature of the electrode and another temperature (e.g., a desired electrode temperature), resulting in an error value. For example, the desired electrode temperature may be input via an input device into field 760, or may be pre-stored by computing device 120.

It should be appreciated that the specific operations illustrated in FIG. 8 provide a particular method of controlling electric fields created by a plurality of electrodes, according to certain embodiments of the present invention. Other sequences of operations may also be performed according to alternative embodiments. For example, alternative embodiments of the present invention may perform the operations outlined above in a different order. Moreover, the individual operations illustrated in FIG. 8 may include multiple sub-operations that may be performed in various sequences as appropriate to the individual operation. Furthermore, additional operations may be added or existing operations removed depending on the particular applications. One of ordinary skill in the art would recognize and appreciate many variations, modifications, and alternatives.

Pattern Switching

Systems and methods and apparatus's as described may perform pattern switching, or differential activation of pairs or groups of electrodes in an array. This is generally done so as to deliver current using two or more different electrode patterns, where the current delivery for each pattern is unique. By changing between unique current patterns, the same or approximately the same amount of current may be applied each electrode over a treatment period, thereby averaging the power throughout a treatment area and thus avoiding or reducing hot spots and cold spots.

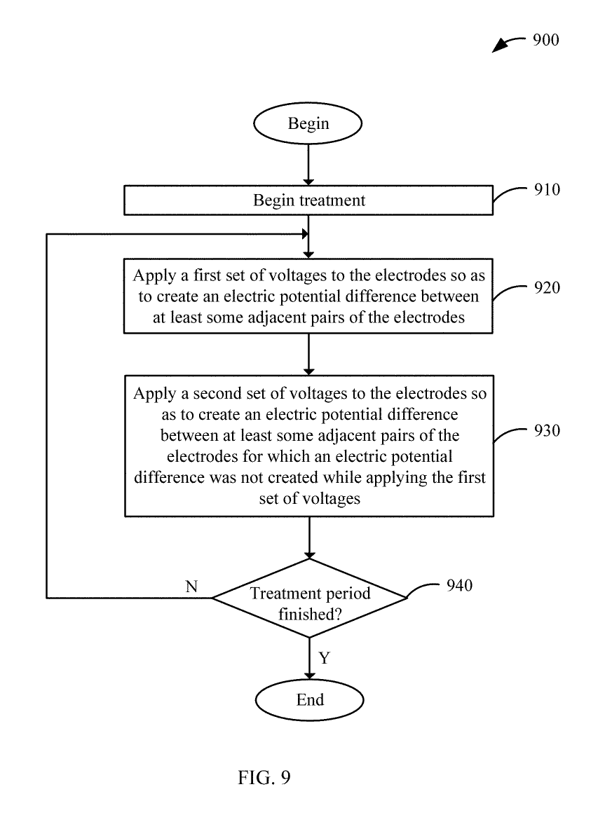

FIG. 9 is flowchart depicting example operations of a method 900 for performing pattern switching according to an embodiment. In operation 910, treatment begins. For example, with reference to FIG. 7B, treatment may begin with activation of start button 722.

In operation 920, a first set of voltages is applied to electrodes in the electrode assembly 170 so as to create an electric potential difference between at least some adjacent pairs of the electrodes. The difference in electric potential may be any suitable difference to generate a desired current flow between the adjacent pairs of electrodes. For example, the difference may be 1V, 5V, 10V, in a range from 1V to 10V, less than 1V, or greater than 10V. The electric potential difference may be generated between any suitable adjacent pairs of electrodes so as to treat a treatment area (e.g., cancerous tissue) of a treatment object (e.g., a human patient). For example, with reference to FIG. 7E, an electric potential difference may be generated between electrodes 1 and 3, and between electrodes 5 and 8. As a result, a current flow pattern may be generated, including a current flow between electrodes 1 and 3 and between electrodes 5 and 8.

In some embodiments, applying the first set of voltages includes creating an absence of an electric potential difference between one or more adjacent pairs of the electrodes. For example, the electric potential difference may be 0V or approximately 0V. With reference to FIG. 7E, while an electric potential difference may be generated between electrodes 1 and 3 and between electrodes 5 and 8, an absence of an electric potential difference may be generated between electrodes 1 and 4 and between electrodes 4 and 8.

In operation 930, a second set of voltages is applied to the electrodes in the electrode assembly 170 so as to create an electric potential difference between at least some adjacent pairs of the electrodes for which an electric potential difference was not created while applying the first set of voltages. For example, with reference to FIG. 7E, in operation 920, an absence of an electric potential difference may have been created between electrodes 1 and 4 and between electrodes 4 and 8. With the second set of voltages, an electrical potential difference may now be created between one or more of those pairs of electrodes.

In some embodiments, the second set of voltages may remove an electric potential difference between at least one of the adjacent pairs of the electrodes that was created while applying the first set of voltages. For example, with reference to FIG. 7E, in operation 920 an electric potential difference may have been created between electrodes 1 and 3 and between electrodes 5 and 8. With the second set of voltages, an absence of an electric potential difference may now be created between one of more of those pairs of electrodes.

In other embodiments, applying the first set of voltages creates an electric potential difference between a first one of the electrodes and one or more first adjacent electrodes, and applying the second set of voltages creates an electrical potential difference between the first one of the electrodes and one or more second adjacent electrodes different than the first adjacent electrodes. For example, with reference to FIG. 7E, in applying the first set of voltages, an electric potential difference may be created between electrodes 1 and 3, and in applying the second set of voltages, an electric potential difference may be created between electrodes 1 and 7. In some cases, applying the second set of voltages removes an electric potential difference between the first one of the electrodes and one or more first adjacent electrodes that was created while applying the first set of voltages. For example, with reference to FIG. 7E, the electric potential difference created between electrodes 1 and 3 while applying the first set of voltages may be removed at the same time the electric potential difference is created between electrodes 1 and 7.

In one embodiment, switching between unique electrode patterns includes creating an electric potential difference between each adjacent pair of electrodes at least once. For example, in operation 920 and with reference to FIG. 7E, the first set of voltages may create an electric potential difference between some of the twenty-nine electrodes shown, and an absence of an electric potential difference between the remainder of the electrodes. The second set of voltages may then create an electric potential difference between the remainder of the electrodes. As a result, a current is passed between all adjacent pairs of electrodes during the course of switching.

In some embodiments, one or more additional sets of voltages may be applied in addition to the first set and the second set. For example, a third set of voltages may be applied. The third set of voltages may have the same or different voltage pattern as the first set and the second set. In one embodiment, the third set of voltages is applied so that, together with application of the first set of voltages and the second set of voltages, an electric potential difference is created between each adjacent pair of electrodes for two of the three sets of voltages. For example, with reference to FIG. 7E, during application of the first and second sets of voltages, a voltage potential may be created between electrodes 1 and 3. Then, by application of the third set of voltages, the voltage potential between electrodes 1 and 3 may be removed. In another embodiment, the third set of voltages creates an electric potential difference for which an electric potential difference was not created while applying the first set or the second set of voltages. For example, with reference to FIG. 7E, during application of the first and second sets of voltages, an absence of voltage potential may be created between electrodes 1 and 3. Then, by application of the third set of voltages, a voltage potential between electrodes 1 and 3 may be created.

For another example, a fourth set of voltages may be applied. The fourth set of voltages may have the same or different voltage pattern as the first set, second set, and third set. In one embodiment, the fourth set of voltages is applied so that, together with application of the first set, second set, and third set of voltages, an electric potential difference is created between each adjacent pair of electrodes for two or three of the sets of voltages. In another embodiment, the fourth set of voltages creates an electric potential difference for which an electric potential difference was not created while applying the first set, second set, or third set of voltages.

In operation 940, computing device 120 determines whether the treatment period is finished. For example, with reference to FIG. 7B, the treatment period may be input by a user via, e.g., test time 712. For another example, the treatment period may be pre-stored in computing device 120. If computing device 120 determines that the treatment period is not finished, processing returns to operation 920, so as to repeat application of the sets of voltages. In contrast, if computing device 120 determines that the treatment period is finished, processing may end. In some embodiments, determination 940 may be performed between one or more operations for applying voltages to the electrodes.

In one embodiment, by repetitively applying multiple sets of voltages to the electrodes, an electric potential difference is created between each adjacent pair of the electrodes at least once over the treatment period. For example, with reference to FIG. 7E, application of a first set of voltages may be applied to create an electric potential difference between at least some adjacent pairs of the electrodes, where an absence of an electric potential difference may remain between at least one adjacent pair of electrodes. Application of a second set of voltages may then be applied, in which an electric potential difference is created for some or all of the adjacent pairs of electrodes for which an absence of an electric potential difference remained as a result of application of the first set of voltages. Accordingly, the second set of voltages may ensure that an electric potential difference is created over each adjacent pair of electrodes. In some cases, even after application of a second set of voltages, there may still remain an absence of an electric potential difference between one or more adjacent pairs of electrodes. Thus, third, fourth, fifth, etc. sets of voltages may be applied to create an electric potential difference over any remaining adjacent pairs of electrodes for which an electric potential difference had not yet been created.

In some embodiments, multiple sets of voltages may be repetitively applied to different subsets of electrodes. For example, with reference to FIG. 7E, multiple sets of voltages may be repetitively applied to a first subgroup of electrodes (e.g., electrodes 1, 3, 4 and 7). Another set of voltages may be repetitively applied to a second subgroup of electrodes (e.g., electrodes 23, 26, 27, and 29). The sets of voltages may be applied to the subgroups simultaneously or at different times with respect to one another, and may create the same or different electric potential differences. In some cases, voltages may only be applied to one of the subgroups. In this fashion, treatment can be localized within an array of electrodes.

It should be appreciated that the specific operations illustrated in FIG. 9 provide a particular method of performing pattern switching, according to certain embodiments of the present invention. Other sequences of operations may also be performed according to alternative embodiments. For example, alternative embodiments of the present invention may perform the operations outlined above in a different order. Moreover, the individual operations illustrated in FIG. 8 may include multiple sub-operations that may be performed in various sequences as appropriate to the individual operation. Furthermore, additional operations may be added or existing operations removed depending on the particular applications. One of ordinary skill in the art would recognize and appreciate many variations, modifications, and alternatives.

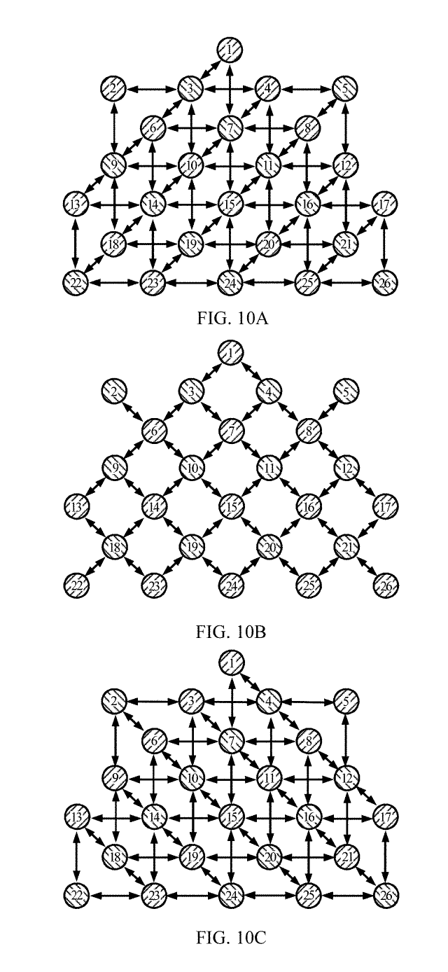

FIGS. 10A to 10C show a sequence of electrode patterns according to an embodiment. FIG. 10A shows a first electrode pattern of a set of electrode patterns and the resulting current flow pattern according to an embodiment. This electrode pattern is generated by applying a set of voltages to the electrodes. In applying the voltages, a first set of electric potentials are created for a first number of electrodes, and a second set of different electric potentials are created for a second number of electrodes. By these differences in electric potential, current flows between electrodes are established.

As shown in FIG. 10A, electrodes 1, 2, 4, 6, 8, 10, 12, 13, 15, 17, 18, 20, 23, and 25 are provided a first electric potential, and electrodes 3, 5, 7, 9, 11, 14, 16, 19, 21, 22, 24, and 26 are provided a second electric potential. Accordingly, numerous electric potential differences and thus current flows between adjacent pairs of electrodes are generated. For example, as shown by the arrowed lines, current flows between electrodes 1 and 3, and between electrodes 1 and 7, and between electrodes 1 and 4, etc.

Although application of the first set of voltages in accordance with the first electrode pattern establishes numerous current flows through a medium, that are there are some paths between electrodes in which only minimal amounts of current flows. The current flows shown by the arrowed lines are significantly larger, such as by an order of magnitude or more, than those paths which are not shown and for which only minimal amounts of current flows. Generally, there is little current flow in a direction from the top left of the electrode array to the bottom right of the electrode array. For example, since there is effectively an absence of electric potential between electrodes 1 and 4 and between electrodes 3 and 7, only a minimal amount of current flows between those electrode pairs. As a result, the medium located between such electrode pairs is not heated as much as between electrode pairs between which a current flows.

Further, while establishing numerous current flows between various electrodes, some electrodes are involved in more current paths than others. For example, electrode number 5 is involved in three current paths; i.e., current flows between electrode 5 and each of electrodes 4, 8, and 12. However, electrode number 2 is involved in only two current paths; i.e., current flows between electrode 2 and each of electrodes 3 and 9. As a result, the medium located at or close to electrode 5 will tend to heat up more quickly than that located at or close to electrode 2 since more current flows in the vicinity of electrode 5 than electrode 2.

FIG. 10B shows a second electrode pattern of a set of electrode patterns and the resulting current flow pattern according to an embodiment. This electrode pattern is generated by applying a second set of voltages different than the first set. In this case, electrodes 1, 6-8, 13-17, and 22-26 are provided at a first electric potential, while electrodes 2-5, 9-12, and 18-21 are provided at a second electric potential.

This electrode pattern addresses the first weakness of the first electrode pattern, in general current paths are created between electrodes in which only minimal amounts of current flowed as a result of the first electrode pattern. That is, current flows are established in the direction from the top left of the electrode array to the bottom right of the electrode array. For example, since there is an electric potential established between electrodes 1 and 4 and between electrodes 3 and 7, relatively significant amounts of current flows between those electrode pairs.

Further, the number of current paths which electrodes are involved in is changed. For example, electrode number 5, which was previously involved in three current paths, is now involved in one current path; i.e., current flows between electrode 5 and 8, rather than between electrode 5 and each of electrodes 4, 8, and 12.

While numerous current flows are established between various electrodes, there are again some paths between electrodes in which only minimal amounts of current flows. Generally, there is little current flow in a vertical direction (e.g., between electrodes 1, 7, 15, and 24) and in a horizontal direction (e.g., between electrodes 9, 10, 11, 12). Further, some electrodes are now involved in only one current path (e.g., electrodes 2 and 5), and some electrodes are now involved in the same number of current paths (e.g., electrodes 2 and 5 are now involved in one current path) but were previously involved in a different number of current paths (e.g., electrodes 2 and 5 were previously involved in 2 and 3 current paths, respectively). The inconsistent number of current paths may result in uneven heating.

FIG. 10C shows a third electrode pattern of a set of electrode patterns and the resulting current flow pattern according to an embodiment. This electrode pattern is generated by applying a third set of voltages different than the first set and the second set. In this case, electrodes 1, 3, 5, 6, 8, 9, 11, 13, 15, 17, 19, 21, 23, and 25 are provided at a first electric potential, while electrodes 2, 4, 7, 10, 12, 14, 16, 18, 20, 22, 24, and 26 are provided at a second electric potential.

This electrode pattern addresses the weakness of the first electrode pattern, in general current flows are established in the direction from the top left of the electrode array to the bottom right of the electrode array. This electrode pattern also addresses the weakness of the second electrode pattern, in that current flows are established in the vertical direction and in the horizontal direction. However, this electrode pattern has its own weakness, in that only minimal current flows are established in the direction from the top right of the electrode array to the bottom left of the electrode array. That being so, this weakness is addressed by the first and second electrode patterns. Accordingly, by application of the sequence of electrode patterns, current flows are established between all adjacent pairs of electrodes, thereby advantageously generating substantially equal amounts of heat through all regions of the medium located proximate to the electrodes.

Further, the number of current paths which electrodes are involved in is changed once again. For example, electrode number 5, which was previously involved in three paths and then one path, is now involved in two paths. Further, electrode number 2, which was previously involved in two paths and one path, is now involved in three paths. As a result, it can be seen that over the course of applying multiple voltage patterns, the amount of current communicated to a given electrode is advantageously averaged out. This is particularly apparent and important for electrodes at the edge of the electrode array, as these electrodes tend to be consistently provided with too many or two few active current paths when only a single electrode pattern is applied.

It should be appreciated that the specific sequence of electrode patterns illustrated in FIGS. 10A to 10C provide a particular sequence of pattern switching, according to certain embodiments of the present invention. Other sequences of pattern switching may also be performed according to alternative embodiments. For example, alternative embodiments of the present invention may perform the pattern switching outlined above in a different order. For another example, alternative embodiments of the present invention may include more or fewer patterns and/or sub-patterns, and may include more or fewer electrodes. One of ordinary skill in the art would recognize and appreciate many variations, modifications, and alternatives.

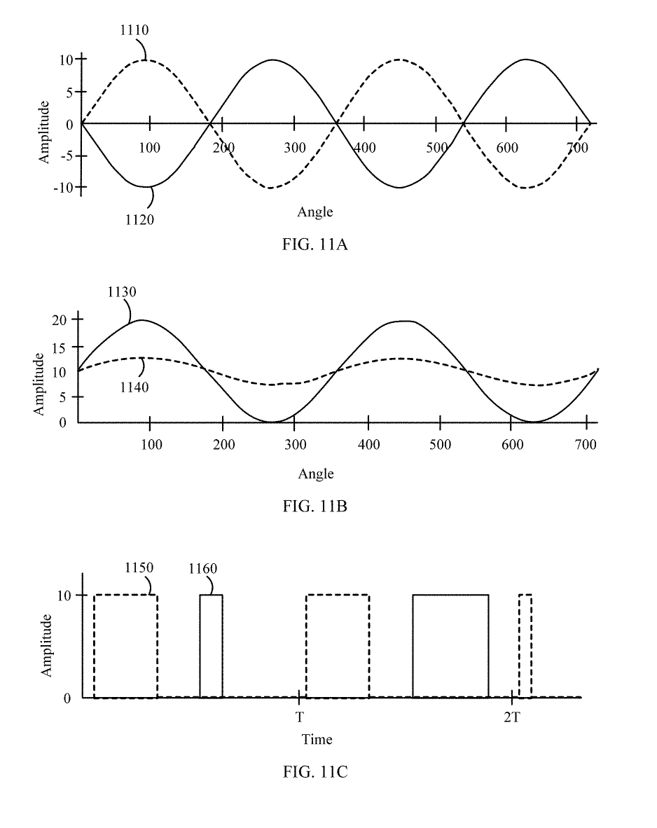

FIGS. 11A to 11C show various techniques for generating a difference in electric potential according to some embodiments. FIG. 11A shows AC signals for generating a difference in electric potential based on a difference in signal polarity or phase.

The AC signals include a first signal 1110 and a second signal 1120. First signal 1110 may be a voltage applied to a first electrode, and second signal 1120 may be a voltage applied to a second electrode. The first and second electrodes may be arranged adjacent to one another, so that differences in electric potential created between the first and second electrodes creates a current flow between those electrodes.

First signal 1110 and second signal 1120 are sinusoidal in this embodiment. However, in other embodiments, different types of analog waveforms may be used, such as square waves, triangular waves, sawtooth waves, etc. First and second signals have maximum amplitudes of 10V in this embodiment. However, in other embodiments, first and second signals may have different maximum amplitudes, such as 3V, 5V, 7V, in a range from 3V to 10V, less than 3V or greater than 10V. First and second signals always have opposite polarities except at their points of intersection. That is, they have opposite polarities except at angles of 180 degrees, 360 degrees, etc. However, in other embodiments, they may not always have opposite polarities. For example, the signals may be phase offset from one another. Further, in other embodiments, they may have points of intersection at other angles.

An amount of current flow between the electrodes may be altered using any suitable technique. In one embodiment, the amplitude of one or more of first signal 1110 and second signal 1120 may be increased or decreased. For example, to increase the difference in electric potential so as to increase a current flow between the electrodes, the maximum amplitude of first signal 1110 may be increased from 10V to 12V. For another example, to increase the difference in electric potential, the maximum amplitude of second signal 1120 may be increased, in addition to or alternatively to an increase in the maximum amplitude of first signal 1110.

FIG. 11B shows AC signals for generating a difference in electric potential based on a difference in signal amplitude. The AC signals include a first signal 1130 and a second signal 1140. First signal 1130 may be a voltage applied to a first electrode, and second signal 1140 may be a voltage applied to a second electrode. The first and second electrodes may be arranged adjacent to one another, so that differences in electric potential created between the first and second electrodes creates a current flow between those electrodes.

First signal 1130 and second signal 1140 are sinusoidal in this embodiment. However, in other embodiments, different types of analog waveforms may be used, such as square waves, triangular waves, sawtooth waves, etc. In this embodiment, the first and second signals have different maximum amplitudes. First signal 1130 has a maximum amplitude of 10V, while second signal 1140 has a maximum amplitude of 2V. First signal 1130 and second signal 1140 may have any suitable different maximum amplitudes. For example, first signal 1130 may have a maximum amplitude of 6V, 8V, 10V, 12V, or in a range of 6V to 12V, or less than 6V or greater than 12V. Second signal 1140 may respectively have a maximum amplitude of 1V, 2V, 3V, 5V, or in a range from 1V to 5V, or less than 1V or greater than 5V.

First and second signals always have the same polarity. That is, in this embodiment, they both always have voltages greater than 0. However, in some embodiments, first and second signals may have a different polarity at some points in time. For example, instead of having a minimum voltage of 0V, first signal 1130 may have a minimum voltage of −2V. Further, in this embodiment, first and second signals have points of intersection at angles of 180 degrees, 360 degrees, etc. However, in other embodiments, they may have points of intersection at other angles.

An amount of current flow between the electrodes may be altered using any suitable technique. In one embodiment, the amplitude of one or more of first signal 1130 and second signal 1140 may be increased or decreased. For example, to increase the difference in electric potential so as to increase a current flow between the electrodes, the maximum amplitude of first signal 1130 may be increased from 10V to 12V. For another example, to increase the difference in electric potential, the maximum amplitude of second signal 1140 may be decreased from 2V to 1V.

FIG. 11C shows AC square wave signals for generating a difference in electric potential based on a pulse width modulation (PWM) of the signals. The AC square wave signals include a first signal 1150 and a second signal 1160. First signal 1150 may be a voltage applied to a first electrode, and second signal 1160 may be a voltage applied to a second electrode. The first and second electrodes may be arranged adjacent to one another, so that differences in electric potential created between the first and second electrodes creates a current flow between those electrodes.

First signal 1150 and second signal 1160 are square voltage pulses in this embodiment. First and second signals have maximum amplitudes of 10V in this embodiment. However, in other embodiments, first and second signals may have different maximum amplitudes, such as 3V, 5V, 7V, in a range from 3V to 10V, less than 3V or greater than 10V. First and second signals always have the same polarity. That is, in this embodiment, they both always have voltages greater than 0. However, in some embodiments, first and second signals may have a different polarity at some points in time.

In this embodiment, the maximum amplitudes of the first and second signals is the same; e.g., 10V. However, in other embodiment, they may be different from one another. For example, first signal 1150 may have a maximum amplitude of 10V, while second signal 1160 may have a maximum amplitude of 5V. In this case, the first and second signals may overlap each other in time, which would also create a difference in electric potential.

The voltage pulses may have any suitable duty cycle, which may be constant or variable. The duty cycle of first signal 1150 may be the same or different than the duty cycle of second signal 1160. Here, in a first time period T, the duty cycles are different. However, in the second time period between T and 2T, the duty cycles are the same. In other embodiments, the duty cycles may be the same for each time period, or different for each time period.

An amount of current flow between the electrodes may be altered using any suitable technique. In one embodiment, the amplitude of one or more of first signal 1130 and second signal 1140 may be increased or decreased. In another embodiment, the duty cycle of one or more of first signal 1130 and second signal 1140 may be increased or decreased. For example, with reference to the first time period T, the duty cycle of second signal 1140 may be increased to, e.g., half of the time period T, so as to increase the amount of time for which a difference in electric potential exists. In yet another embodiment, where the amplitude of the first and second signals in a given time period is different, the voltage pulses may overlap with one another, thereby creating a difference in electric potential not only where the voltage pulses do not overlap but also where they do overlap.

It should be appreciated that the specific techniques for generating a difference in electric potential illustrated in FIGS. 11A to 11C provide particular examples for generating such a difference according to certain embodiments of the present invention. Other techniques for generating a difference in electric potential may also be used according to alternative embodiments. For example, techniques may use polarity differences, amplitude differences, phase differences, etc. in AC and/or DC signals having various types of waveforms in order to create such a difference. One of ordinary skill in the art would recognize and appreciate many variations, modifications, and alternatives.

Customized Feedback Control Loop

Systems and methods and apparatus's as described may use a customized feedback control loop to determine voltages to apply to electrodes in an array. This is generally done so as improve a user's control of current delivery and thus a user's control over tissue heating. For example, the temperature of electrodes adjacent to a controlled electrode may be used in determining a voltage to apply to the controlled electrode. In so doing, an overheating of the adjacent electrode may be controlled. For another example, the voltages of electrodes other than a controlled electrode may be used in determining a voltage to apply to the controlled electrode. In so doing, an increase or decrease in current delivery to the controlled electrode may be more accurately controlled.

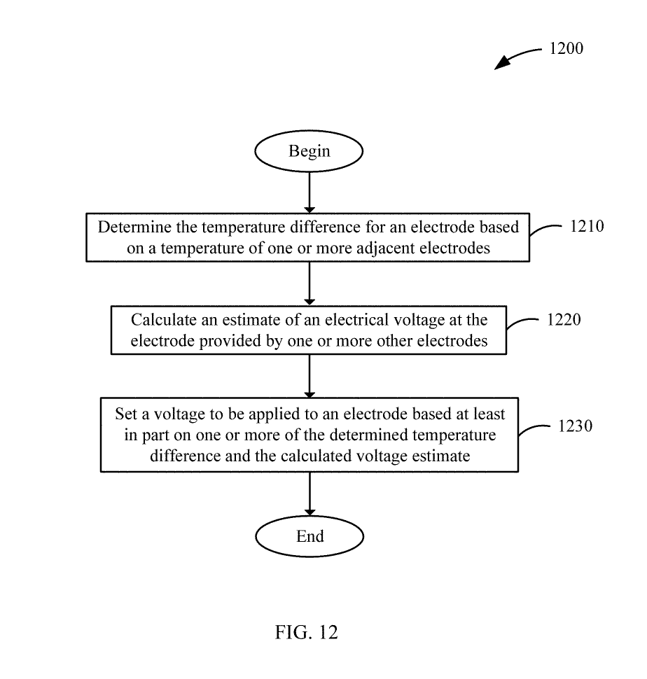

FIG. 12 is a flowchart depicting example operations of a customized feedback control process 1200 according to a first embodiment. The customized feedback control process 1200 may be performed by any suitable device, such as computing device 120 discussed with reference to FIG. 1B, and may include one or more of the following operations.

In operation 1210, computing device 120 determines a temperature difference for an electrode based on a temperature of an adjacent electrode. In one embodiment, the temperature difference may be the difference between the electrode temperature and a desired electrode temperature (e.g., a desired temperature input via an input device into field 760, or may be pre-stored by computing device 120). The desired electrode temperature may represent a maximum electrode temperature desired by, for example, a medical practitioner. However, if such a temperature difference is the sole difference used to determine the electrode voltage, a temperature of the electrode may be increased without reference to or concern for the temperature of adjacent electrodes. Where an adjacent electrode has already attained a desired temperature, blindly increasing the voltage and temperature of the electrode may undesirably cause a temperature of the adjacent electrode to exceed the desired temperature.

Accordingly, in some embodiments, the temperature difference used to determine a voltage for an electrode may take into consideration a temperature of an adjacent electrode. By taking the temperature of the adjacent electrode into consideration, a temperature of the electrode may not be blindly increased in an attempt to reach a desired temperature, thereby reducing the likelihood that a temperature of the adjacent electrode exceeds a desired temperature of the adjacent electrode.

In one embodiment, a maximum electrode temperature may be set to be less than the desired electrode temperature. The temperature difference may then be set as the difference between the temperature of the electrode and the set maximum electrode temperature. The maximum electrode temperature may represent a maximum temperature of the electrode as identified by computing device 120 for the purposes of determining a voltage to apply to the electrode. By setting the maximum electrode temperature to be less than the desired electrode temperature, a current flow to one or more adjacent electrodes may be reduced compared to what it otherwise may have been, thereby advantageously preventing an excess amount of heat to be generated proximate to the one or more adjacent electrodes.

The maximum electrode temperature of an electrode may be set to be less than a desired electrode temperature using one or more of a variety of techniques. In one embodiment, a temperature of an adjacent electrode may be determined and used to set the maximum electrode temperature. The difference between the temperature of the electrode and the newly set maximum electrode temperature may then be used to determine a voltage to apply to the electrode. In another embodiment, a temperature of a plurality of adjacent electrodes may be determined. If the temperature of one or more of the adjacent electrodes is greater than a temperature of the electrode, one of the temperatures of the adjacent electrodes may be set as the maximum electrode temperature. In some embodiments, a highest temperature of the one or more adjacent electrodes may be identified and used.

In another embodiment, a temperature of an electrode may be set to be greater than the actual temperature of the electrode. The temperature difference may then be set as the difference between the set electrode temperature and a desired electrode temperature. By setting the electrode temperature to be greater than the actual temperature of the electrode, a current flow to one or more adjacent electrodes may be reduced compared to what it otherwise may have been, thereby advantageously preventing an excess amount of heat to be generated proximate to the one or more adjacent electrodes.

The temperature of an electrode may be set to be greater than the actual temperature of the difference using one or more of a variety of techniques. In one embodiment, a temperature of an adjacent electrode may be determined and used to set the temperature of the electrode. The difference between the newly set temperature of the electrode and the desired temperature may then be used to determine a voltage to apply to the electrode. In another embodiment, a temperature of a plurality of adjacent electrodes may be determined. If the temperature of one or more of the adjacent electrodes is greater than a temperature of the electrode, one of the temperatures of the adjacent electrodes may be set as the electrode temperature. In some embodiments, a highest temperature of the one or more adjacent electrodes may be identified and used.

One skilled in the art would recognize the numerous variations of the above-described techniques and other possibilities for setting the temperature difference for an electrode, and all such variations are within the scope of the present disclosure. For example, the temperature of the electrode and/or the desired electrode temperature may be set to a fraction of the adjacent electrode temperature (e.g., 50%, 70%, 90%, in the range from 50% to 90%, less than 50% or greater than 90%) or to a multiple of the adjacent electrode temperature (e.g., 110%, 150%, 200%, in the range of 110% to 200%, less than 110% or greater than 200%). For another example, the temperature of the electrode and/or desired electrode temperature may be set to an average temperature of one or more adjacent electrodes, or an average temperature of all other electrodes, or an average temperature of select electrodes (e.g., those electrodes having a temperature exceeding the desired temperature). For yet another example, both the temperature of the electrode and the desired electrode temperature may be set based and/or adjusted based on the adjacent electrode temperature.

In some embodiments, the temperature of an electrode may be determined at least in part based on the temperature of one or more adjacent electrodes only during a portion of a treatment period. For example, the temperature of the adjacent electrodes may be used while the temperature of the electrodes ramps up to their desired temperature. In other embodiments, the temperature of the electrode may be determined at least in part based on the temperature of one or more adjacent electrodes during the entire treatment period.

In operation 1220, computing device 120 calculates an estimate of an electrical voltage at the electrode provided by one or more other electrodes. The estimate may be an estimate of an average voltage at the electrode provided by one or more other electrodes. By using an estimated voltage provided at an electrode by other electrodes, it is possible to predict what electrode voltage would result in a high or low current flow between the electrode and other electrodes, thereby increasing the accuracy of heat generation. Specifically, with pattern switching and individual control over electrical voltage and phase, the voltage potential at each electrode location may always be changing. As a result, decreasing the voltage at an electrode (e.g., decreasing the voltage to zero) may not necessarily decrease current flow to or from the electrode if the surrounding electrodes are in phase and at a higher voltage. Accordingly, using an estimated voltage at the electrode rather than zero may advantageously compensate for such complications.

The estimated voltage provided at the electrode may be determined using one or more of a variety of techniques. In one embodiment, a voltage of a plurality of adjacent electrodes is identified. The voltage of each adjacent electrode may then be adjusted based on a distance of the adjacent electrode from the electrode. For example, the voltage may be multiplied by a factor representative of distance. The adjusted voltages may then be averaged by, for example, summing the voltages and dividing the result by the number of adjacent electrodes. The estimated voltage potential at that electrode location may then be used to determine an electrical signal to be applied to the electrode.

In another embodiment, a voltage of all other electrodes may be identified. For example, the all other electrodes may include all of the electrodes in the electrode array being controlled other than an electrode for which the voltage is being determined. Similar to the embodiment discussed above, the average voltage of all of the other electrodes may be calculated and then used to determine an electrical voltage to be applied to the electrode.

One skilled in the art would recognize the numerous variations of the above-described techniques and other possibilities for calculating and using an average voltage to determine a voltage to be applied to an electrode, and all such variations are within the scope of the present disclosure. For example, the average voltage of adjacent electrodes as well as additional (but not all other) electrodes may be calculated and used.

In operation 1230, computing device 120 sets a voltage to be applied to an electrode based at least in part on one or more of the determined temperature difference and calculated voltage estimate. For example, the voltage to be applied to an electrode may be set using the temperature difference determined in operation 1210. For another example, the voltage to be applied to an electrode may be set using the calculated voltage estimate in operation 1220. In some cases, both the temperature difference and the voltage estimate may be used to set the voltage to be applied to an electrode.

It should be appreciated that the specific operations illustrated in FIG. 12 depict example operations of a customized feedback control process, according to certain embodiments of the present invention. Other sequences of operations may also be performed according to alternative embodiments. For example, alternative embodiments of the present invention may perform the operations outlined above in a different order. Moreover, the individual operations illustrated in FIG. 12 may include multiple sub-operations that may be performed in various sequences as appropriate to the individual operation. Furthermore, additional operations may be added or existing operations removed depending on the particular applications. One of ordinary skill in the art would recognize and appreciate many variations, modifications, and alternatives.

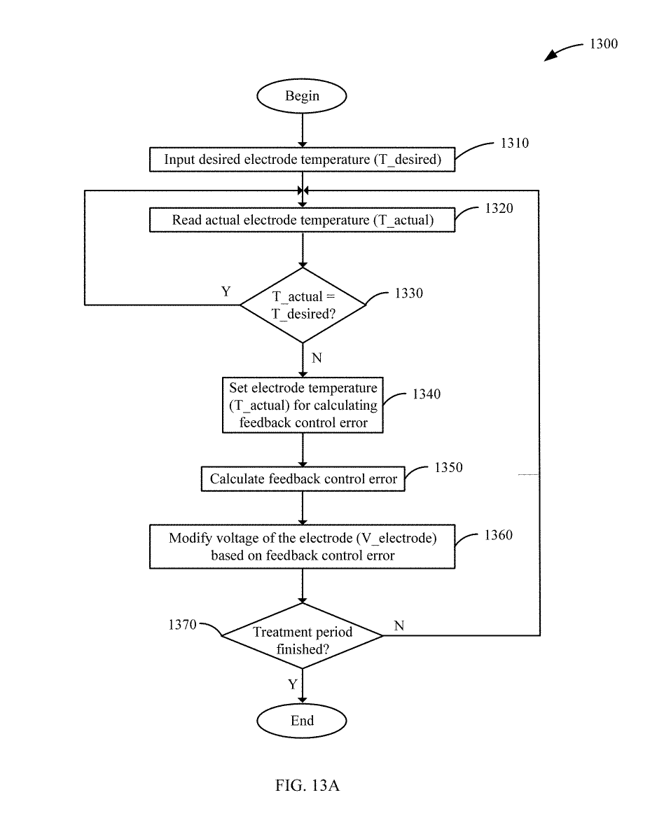

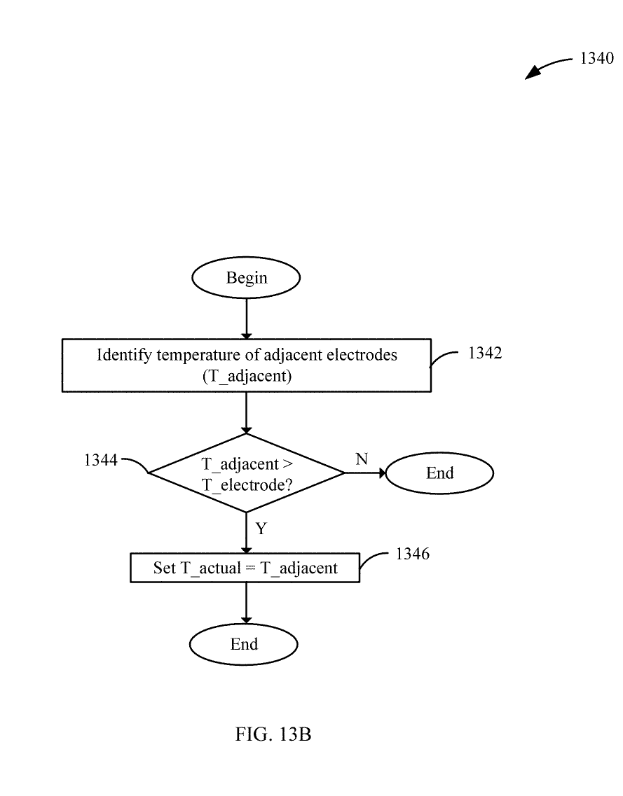

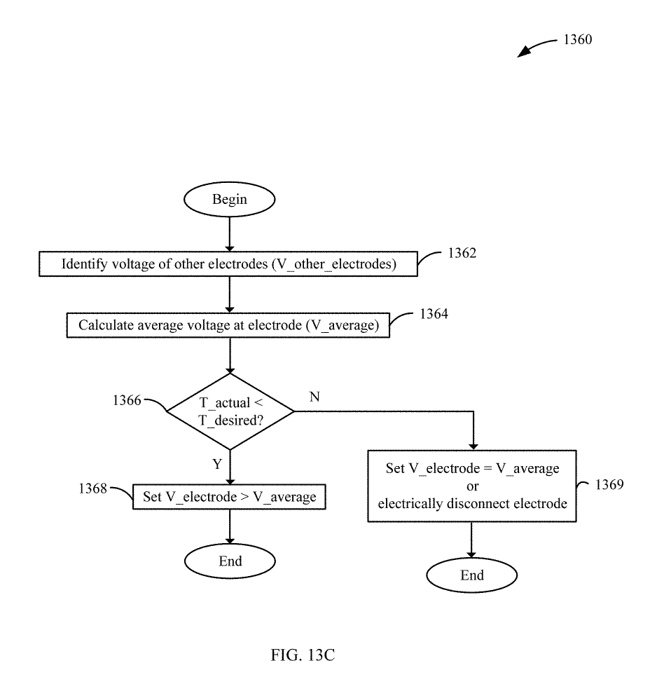

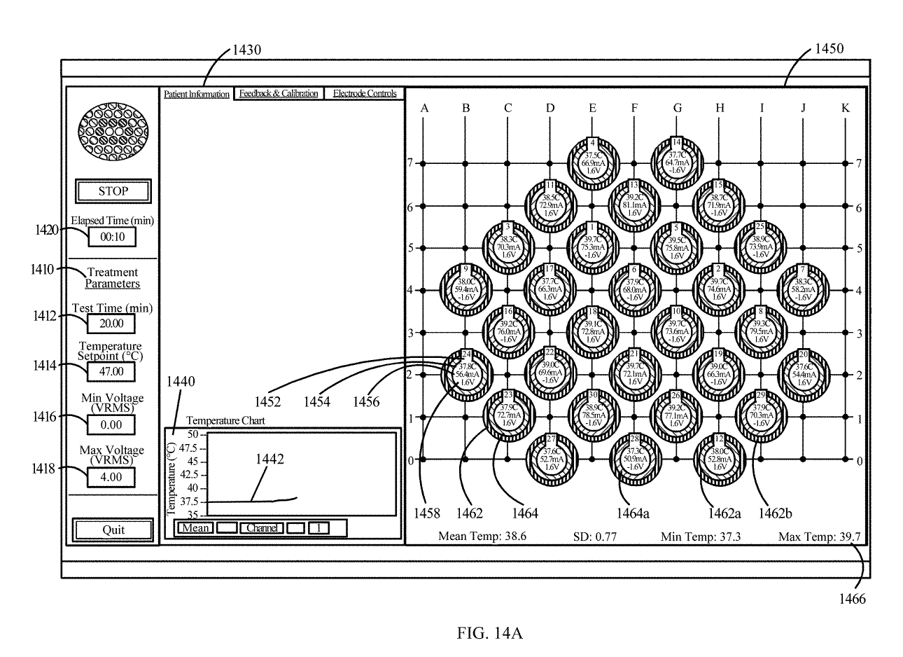

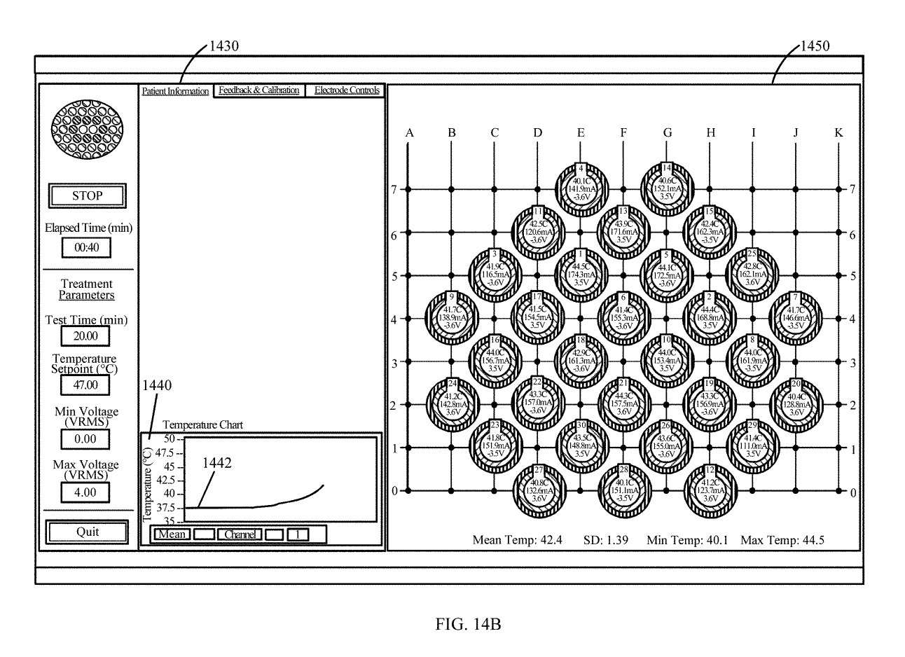

FIG. 13A is a flowchart depicting example operations of a customized feedback control process 1300 according to a second embodiment. The customized feedback control process 1300 may be performed by, for example, computing device 120 (discussed with reference to FIG. 1B), and may operate to control a voltage applied to, for example, electrodes of needle electrode assembly 170. The operations may be performed for one or more electrodes in any suitable order. For example, the operations may be performed for all of the electrodes of needle electrode assembly 170 to be controlled to apply an electric field to a treatment area. The operations may be performed simultaneously with or separate from a pattern switching such as that discussed with reference to FIG. 8.

In operation 1310, a desired electrode temperature (T_desired) is input. In one embodiment, the desired electrode temperature may be input via an input device into field 760 discussed with reference to FIG. 7D. In another embodiment, the desired electrode temperature may be pre-stored by computing device 120. The desired electrode temperature may be stored in storage 124. The desired electrode temperature may represent a maximum temperature of an electrode desired by, for example, a medical practitioner.

In operation 1320, the actual temperature (T_actual) of the electrode is read. For example, the electrode may include temperature sensor 330 discussed with reference to FIG. 3B. The measurement from temperature sensor 330 may be read by amplifier board 140 and communicated to computing device 120. For another example, an external temperature sensor may be provided proximate the electrode, and the measurement from the external temperature sensor may be communicated to computing device 120 using any suitable communication path. The actual temperature may indicate a current temperature of the electrode or in an immediate vicinity of the electrode.

In operation 1330, a determination is made as to whether the actual temperature (T_actual) is equal to the desired temperature (T_desired). If it is determined that T_actual is equal to T_desired, processing may return to operation 1320. If it is determined that T_actual is not equal to T_desired, for example, T_actual is greater or less than T_desired, then processing may continue with operation 1340.

In operation 1340, the electrode temperature is set for the purposes of calculating feedback control error. Various techniques may be used for setting the electrode temperature for the purposes of calculating feedback control error, some of which were discussed with reference to operation of 1210 of FIG. 12, and another of which is subsequently discussed with reference to FIG. 13B.

In operation 1350, a feedback control error is calculated. Feedback control error is indicative of a difference between T_actual and T_desired, where the customized feedback control process 1300 seeks to minimize the feedback control error. The feedback control error may be any value representative of the difference between T_actual and T_desired. For example, the feedback control error may be equal to a difference between an actual temperature of the electrode and a desired temperature of the electrode. For another example, the feedback control error may be equal to a difference between the actual temperature of the electrode and a temperature of an adjacent electrode. For another example, the feedback control error may be equal to a difference between the average temperature of one or more adjacent electrodes and the desired temperature of the electrode, or the difference between the actual electrode temperature and the average temperature of one or more adjacent electrodes.

Feedback control error may also be based on one or more additional indicators of error. For example, a constant may be added or removed to a calculated temperature difference. For another example, a derivative and/or integral of one or more temperature differences over time may be added or removed. For yet another example, multiple differences (either the same or different temperature differences) may be summed, averaged, or the like, with the result either used as the feedback control error, added to other error calculations, or removed from other error calculations. One of ordinary skill in the art would recognize and appreciate many variations, modifications, and alternatives for calculating the feedback control error.

In one embodiment, the feedback control error is calculated as the difference between T_actual and T_desired. T_actual may be set in accordance with operation 1340 and as discussed with reference to FIG. 13B. Accordingly, T_actual may be equal to the actual temperature of the electrode, or may be equal to a temperature of an adjacent electrode (T_adjacent).

In operation 1360, a voltage of the electrode (V_electrode) is modified based on the feedback control error. Various techniques may be used for modifying the voltage of the electrode, some of which were discussed with reference to operation 1210 of FIG. 12, and another of which is subsequently discussed with reference to FIG. 13C. As a result of operation 1360, a new application voltage to be applied to the electrode is determined. The newly determined voltage may be applied to the electrode using, for example, amplifier board 140.

In operation 1370, a determination is made as to whether a treatment period is finished. In one embodiment, the treatment period may be input via an input device into field 712 discussed with reference to FIG. 7B. In another embodiment, the treatment period may be pre-stored by computing device 120. Information indicating the treatment period may be stored in storage 124. The treatment period may represent a duration for which electrodes in, for example, needle electrode assembly 170 operate to apply an electric field. The determination may be made by comparing an elapsed treatment time to the stored treatment period.

FIG. 13B is a flowchart depicting example operations for setting an electrode temperature in accordance with operation 1340 of FIG. 13A. In operation 1342, a temperature of one or more adjacent electrodes (T_adjacent) is identified. The temperature may be identified by reading the temperature of the one or more adjacent electrodes similar to reading an electrode temperature discussed with reference to operation 1320.

As discussed throughout this description, adjacent electrodes may be any or all electrodes within a suitable vicinity of a subject electrode (e.g., an electrode for which a voltage to be applied thereto is determined). In one embodiment, the adjacent electrodes may be the nearest electrodes in each direction. For example, with reference to FIG. 7E, electrode 11 may be the electrode for which a voltage to be applied thereto is determined. The adjacent electrodes may include electrodes 4, 7, 8, 10, 12, 15, 16, and 20. In another embodiment, the adjacent electrodes may be only a limited number of closest electrodes. For example, considering electrode 11 again, the adjacent electrodes may include only electrodes 7, 8, 15, and 16, since they all are an equal distance from electrode 11 and are all a minimal distance from electrode 11 compared to other electrodes.

In operation 1344, it is determined whether the temperature of the adjacent electrode (or electrodes) is not greater than a temperature of the electrode under consideration. If it is determined that the temperature of the adjacent electrode (or electrodes) is not greater than a temperature of the electrode under consideration, then processing for setting the electrode temperature may end and thus processing may return to operation 1350. In such a case, the temperature of the electrode is not adjusted for the purposes of calculating feedback control error, and thus the actual temperature of the electrode is used to calculate the feedback control error. In one embodiment, it is determined that the temperature of the adjacent electrode is not greater than a temperature of the electrode only if a temperature of all of the adjacent electrodes is not greater than a temperature of the electrode.

If it is determined that the temperature of the adjacent electrode (or electrodes) is greater than a temperature of the electrode, then processing may continue with operation 1346. In operation 1346, the temperature of the electrode (T_actual) may be set equal to the temperature of the adjacent electrode. Processing for setting the electrode temperature may then end and return to operation 1350. In such a case, the temperature of the electrode is adjusted for the purposes of calculating feedback control error. That is, instead of using the actual temperature of the electrode to calculate the feedback control error, a temperature of an adjacent electrode may be used in place of the temperature of the electrode to calculate the feedback control error.

If a temperature of one or more adjacent electrodes is greater than a temperature of the electrode, any suitable adjustment may be made to the temperature of the electrode. In one embodiment, the maximum temperature of the adjacent electrodes is determined, and T_actual is replaced with this maximum temperature. In another embodiment, an average temperature of all of the temperatures for adjacent electrodes exceeding the temperature of the electrode is determined, and T_actual is replaced with this average temperature. One skilled in the art would recognize the numerous variations of the above-described techniques and other possibilities for adjusting the temperature of the electrode, and all such variations are within the scope of the present disclosure.

FIG. 13C is a flowchart depicting example operations for modifying a voltage of an electrode (V_electrode) in accordance with operation 1360 of FIG. 13A. In operation 1362, a voltage of other electrodes (V_other_electrodes) is identified. The other electrodes may include any suitable electrodes of the controlled electrode array. For example, the other electrodes may be adjacent to an electrode for which a voltage is to be determined. For another example, the other electrodes may include all of the controlled electrodes of the array other than the electrode for which a voltage is to be determined. Further, the voltage of the other electrodes may be identified using one or more of a variety of techniques. For example, information indicating a current voltage being applied to the other electrodes may be stored in storage 124 and subsequently read by processor 122.

In operation 1364, an estimated voltage (V_estimated) at the electrode is calculated. The estimated voltage at the electrode may be determined using one or more of a variety of techniques. In one embodiment, the identified voltage of the other electrodes may be adjusted based on a distance of the other electrodes from the electrode. For example, the voltage may be multiplied by a factor representative of distance. The adjusted voltages may then be averaged by, for example, summing the voltages and dividing the result by the number of adjacent electrodes. The average of the adjusted voltages may then be used as the estimated voltage at the electrode.

In operation 1366, a determination is made as to whether the actual temperature of the electrode (T_actual) is less than the desired temperature (T_desired). The actual temperature of the electrode may be the actual temperature of the electrode as discussed with reference to operation 1330, or it may be set to a different value as discussed with reference to operation 1340.

If it is determined that T_actual is less than T_desired, then processing continues to operation 1368, where V_electrode is set to be greater than V_estimated. In some embodiments, V_electrode may be set to be lower than V_estimated. As a result of creating a difference in voltage between V_estimated and V_electrode, a current may be caused to flow to the electrode, thereby increasing a temperature of the electrode. The difference in voltage between V_estimated and V_electrode may be determined based on the feedback control error calculated in operation 1350. For example, where the feedback control error indicates a large temperature difference, V_electrode may be set to create a large difference in voltage with respect to V_estimated, so as to create a large current flow to the electrode and thus heating of the electrode. Where the feedback control error indicates a small temperature difference, V_electrode may be set to create a small difference in voltage with respect to V_estimated, so as to create a small current flow to the electrode and thus small or reduced heating of the electrode.

If it is determined that T_actual is not less than T_desired, then processing continues to operation 1369, where V_electrode is set approximately equal to V_estimated or is electrically disconnected. As a result of setting V_electrode approximately equal to V_estimated, a current flow to the electrode may be reduced, thereby maintaining or reducing a temperature of the electrode. Similarly, as a result of electrically disconnecting the electrode, a current flow to the electrode may be reduced, thereby maintaining or reducing a temperature of the electrode. In some embodiments, one or more electrodes adjacent to or in the vicinity of the electrode may also be electrically disconnected. For example, all of the electrodes surrounding an electrode for which T_actual is greater than or equal to T_desired may be electrically disconnected. In some cases, electrically disconnecting adjacent electrodes may be performed simultaneously with disconnecting the electrode. In other cases, the adjacent electrodes may be disconnected only if the electrode continues to overheat for a predetermined time.

It should be appreciated that the specific operations illustrated in FIGS. 13A to 13C provide particular operations of a customized feedback control process, according to certain embodiments of the present invention. Other sequences of operations may also be performed according to alternative embodiments. For example, alternative embodiments of the present invention may perform the operations outlined above in a different order. Moreover, the individual operations illustrated in FIGS. 13A to 13C may include multiple sub-operations that may be performed in various sequences as appropriate to the individual operation. Furthermore, additional operations may be added or existing operations removed depending on the particular applications. One of ordinary skill in the art would recognize and appreciate many variations, modifications, and alternatives.

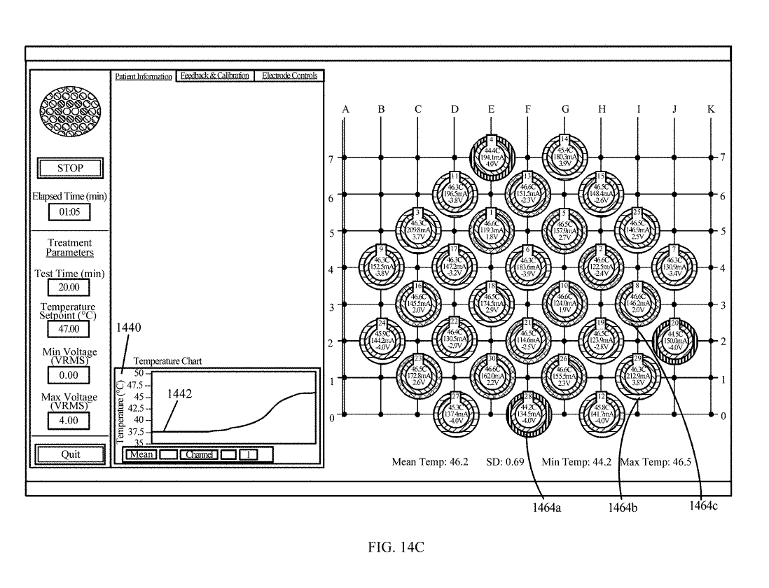

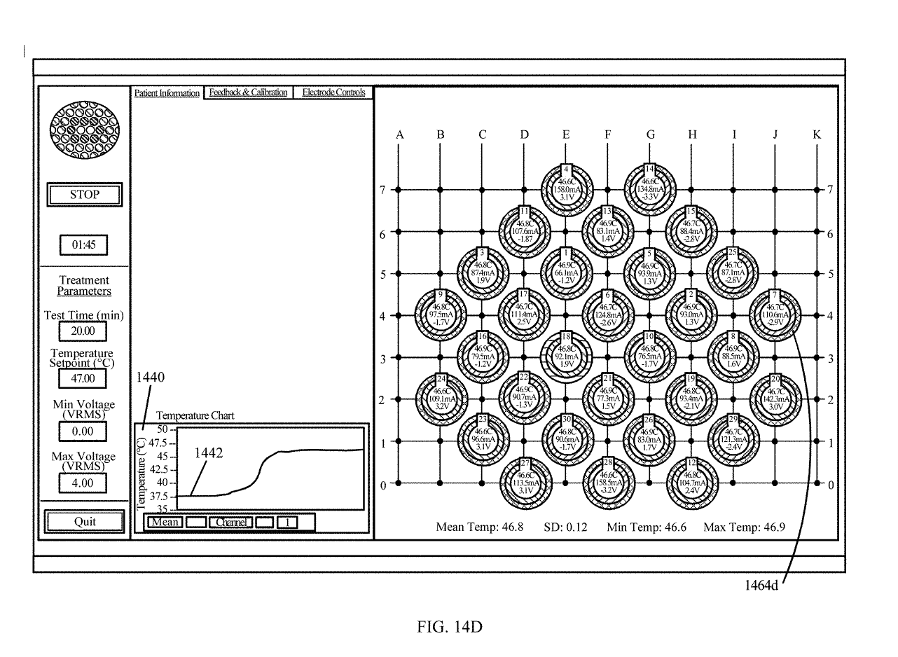

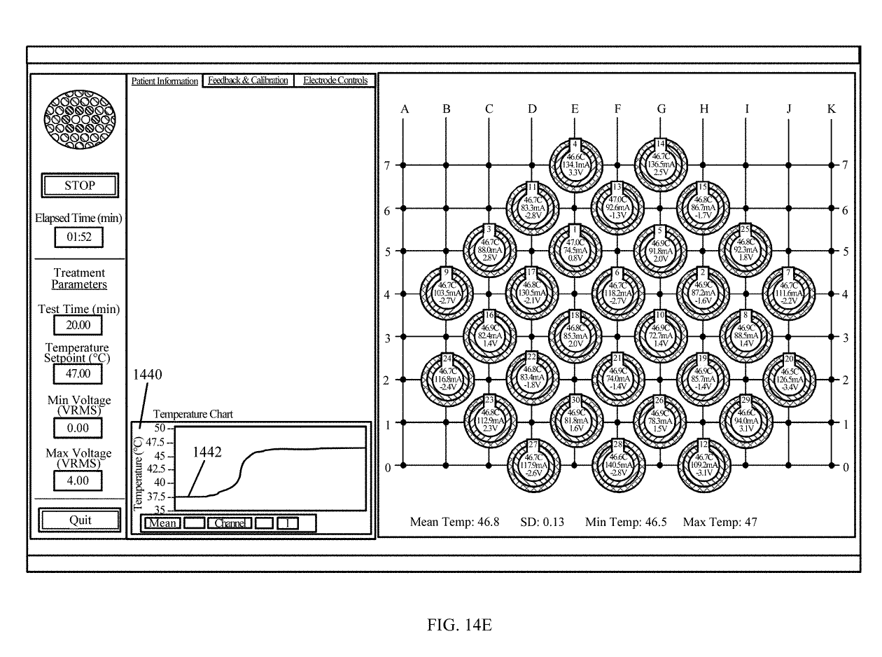

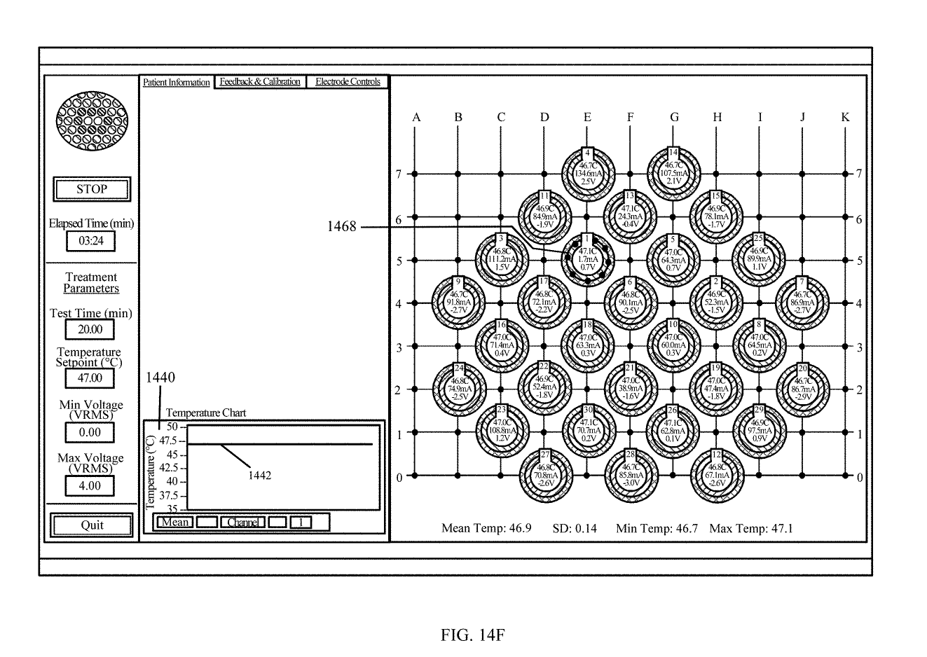

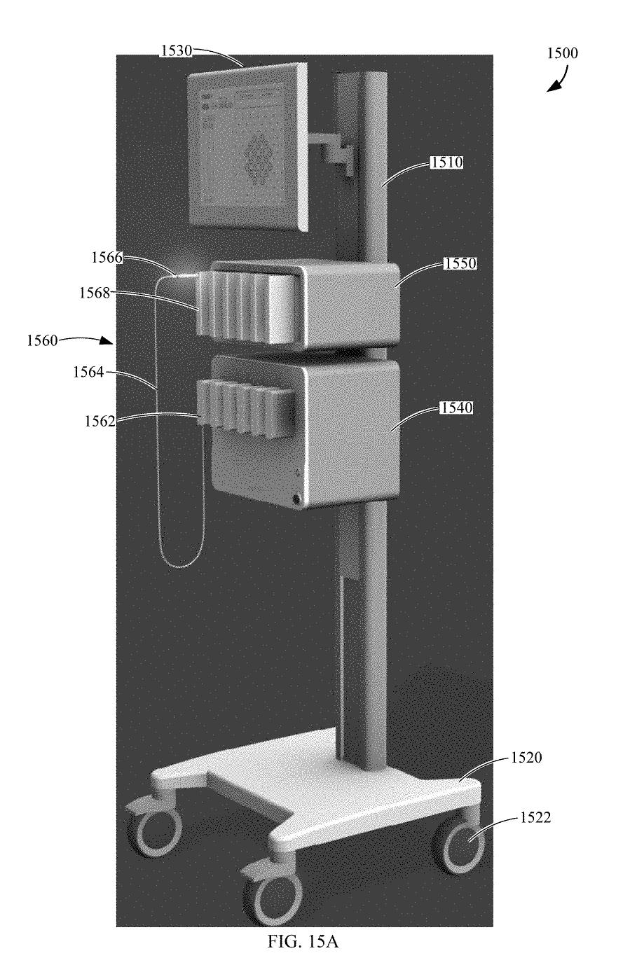

FIGS. 14A to 14F show the voltages and temperatures of a plurality of electrodes over a portion of a treatment period. Pattern switching and a customized feedback control loop as previously discussed may be performed for the electrodes. Two sequences of three electrode patterns are shown, where each electrode sequence includes the three electrode patterns discussed with reference to FIGS. 10A to 10C.

FIG. 14A shows the voltages and temperatures of a plurality of electrodes for a time instance in which a first electrode pattern is applied, along with other treatment-related information. Treatment parameters 1410, an elapsed time value 1420, a patient information element 1430, a temperature chart 1440, and an electrode status element 1450, similar to those discussed with reference to FIG. 7A, are all shown and may be displayed to a user via, for example, display device 130 discussed with reference to FIG. 1B.

In this embodiment, treatment parameters 1410 include a test time 1412 (i.e., treatment period) of 20 minutes, a desired electrode temperature 1414 of 47 degrees Celcius, a minimum voltage 1416 of 0 V, and a maximum voltage 1418 of 4 V. An elapsed time value 1420 shows an elapsed treatment time of 10 seconds. Patient information element 1430 includes a temperature chart 1440 showing a mean temperature 1442 of the electrodes. Electrode status element 1450 shows various information concerning each electrode, including an electrode identifier 1452, a current temperature 1454, a current electrical current 1456, a current electrical voltage 1360, a polarity indicator 1462, and a relative temperature indicator 1464, similar to those discussed with reference to FIG. 7F. Electrode status element 1450 also shows summary statistics information 1466 similar to that discussed with reference to FIG. 7E.

As shown in FIG. 14A, a first electrode pattern is applied at an elapsed time of 10 seconds. The first electrode pattern includes positive voltages 1.6V being applied to each of electrodes 2-5, 8, 12, 13, 17, 18, 20, 21, 23, 24, 26, and 27, and negative voltages of 1.6V being applied to each of electrodes 1, 6, 7, 9-11, 14-16, 19, 22, 25, and 28-30. At this time, the electrodes have temperatures ranging from 37.3 degrees to 39.7 degrees, and currents ranging from 50.9 mA to 81.8 mA. While the desired temperature is 47 degrees, the mean temperature is only 38.6 degrees. Polarity indicators 1462 show whether the electrodes have a positive or a negative voltage being applied to them. For example, polarity indicator 1462a indicates application of a positive voltage, whereas polarity indicator 1462b indicates application of a negative voltage. Relative temperature indicators 1464 show the temperature of the electrodes relative to the desired temperature. For example, relative temperature indicator 1464a shows that the temperature of electrode 28 is less than 45.0 degrees. Summary statistics information 1466 shows the mean temperature, standard deviation of the temperature, minimum temperature, and maximum temperature, of the electrodes.

FIG. 14B shows the voltages and temperatures of a plurality of electrodes for a time instance in which a second electrode pattern is applied. Here, a second electrode pattern is applied at an elapsed time of 40 seconds. The second electrode pattern includes positive voltages being applied to each of electrodes 1, 2, 10, 12-14, 16, 17, 20, 21, 24, 25, 27, 29, and 30, and negative voltages being applied to each of electrodes 3-9, 11, 15, 18, 19, 22, 23, 26, and 28. The voltages range in amplitude from 3.5 to 3.6V. Further, at this time, the electrodes have temperatures ranging from 40.1 degrees to 44.5 degrees, and currents ranging from 111.0 mA to 174.3 mA. While the desired temperature is 47 degrees, the mean temperature is only 42.4 degrees. The relative temperature indicators 1464 show that the temperature of all of the electrodes is less than 45.0 degrees. Further, temperature chart 1440 shows the mean temperature 1442 at the elapsed time of 40 seconds as well as the history of the mean temperature for the duration of the treatment period.

FIG. 14C shows the voltages and temperatures of a plurality of electrodes for a time instance in which a third electrode pattern is applied. Here, a third electrode pattern is applied at an elapsed time of 1 minutes and 5 seconds. The third electrode pattern includes positive voltages being applied to each of electrodes 1, 3-5, 8, 10, 14, 16, 18, 23, 25, 26, 29, and 30, and negative voltages being applied to each of electrodes 2, 6, 7, 9, 11-13, 15, 17, 0.19-22, 24, and 27. The voltages range in amplitude from 1.9V to 4.0V. Further, at this time, the electrodes have temperatures ranging from 44.2 degrees to 46.6 degrees, and currents ranging from 114.6 mA to 212.9 mA. While the desired temperature is 47 degrees, the mean temperature is now 46.2 degrees. The relative temperature show multiple relative ranges of temperature. For example, relative temperature indicator 1464a shows that the temperature of electrode 28 is less than 45.0 degrees, where relative temperature indicator 1464b shows that the temperature of electrode 29 is greater than 45.0 degrees and less than 46.5 degrees. Relative temperature indicator 1464c shows that the temperature of electrode 8 is greater than 46.5 degrees. In some cases, relative temperature indicator 1464c may show that the temperature is less than the desired temperature (e.g., 47 degrees) or a temperature close to the desired temperature (e.g., 47.5 degrees), and another relative temperature indicator (not shown) may show that the temperature exceeds the desired temperature or the temperature close to the desired temperature.

FIG. 14D shows the voltages and temperatures of a plurality of electrodes for another time instance in which the first electrode pattern is applied. Here, the first electrode pattern is applied again, this time at an elapsed time of 1 minutes and 45 seconds. At this point, the voltages applied by the electrodes range in amplitude from 1.2V to 3.3V. Further, at this time, the electrodes have temperatures ranging from 46.6 degrees to 46.9 degrees, and currents ranging from 66.1 mA to 158.5 mA. The mean temperature is now 46.8 degrees. The relative temperature show multiple relative ranges of temperature, all nearly at the desired temperature. For example, relative temperature indicator 1464d shows that the temperature of electrode 7 is at least 46.5 degrees.

FIG. 14E shows the voltages and temperatures of a plurality of electrodes for another time instance in which the second electrode pattern is applied. Here, the second electrode pattern is applied again, this time at an elapsed time of 1 minutes and 52 seconds. At this point, the voltages applied by the electrodes range in amplitude from 0.8V to 3.4V. Further, at this time, the electrodes have temperatures ranging from 46.5 degrees to 47.0 degrees, and currents ranging from 74.0 mA to 140.5 mA. The mean temperature is 46.8 degrees.

FIG. 14F shows the voltages and temperatures of a plurality of electrodes for another time instance in which the third electrode pattern is applied. Here, the third electrode pattern is applied again, this time at an elapsed time of 3 minutes and 24 seconds. At this point, the voltages applied by the electrodes range in amplitude from 0.1V to 3.0V. Further, at this time, the electrodes have temperatures ranging from 46.7 degrees to 47.1 degrees, and currents ranging from 1.7 mA to 111.2 mA. The mean temperature is 46.9 degrees. Further, a disconnect indicator 1468 is shown for electrode 1, indicating that the electrode has been electrically disconnected or that a voltage of the electrode has been set based on an estimated electrical voltage provided at electrode 1 by other electrodes. For example, as discussed with reference to operation 1220 of FIG. 12, an estimate of the voltage at electrode 1 may be determined based on a voltage of other electrodes (e.g., adjacent electrodes 3-6, 11, 13, 17, and 18). In this case, the estimated voltage at electrode 1 may be equal to 0.7V, and thus a voltage of electrode 1 may be set at 0.7V. As a result, a voltage potential between electrode 1 and the adjacent electrodes is minimized so as to reduce the amount of current flowing to/from electrode 1, and thus ideally reduce the temperature from 47.1 degrees to the desired 47.0 degrees.

It should be appreciated that the specific sequence of electrode patterns illustrated in FIGS. 14A to 14F show a particular sequence of pattern switching being repetitively applied to a treatment area, according to certain embodiments of the present invention. Although three unique electrode patterns are shown at specific instances in time, it should be recognized that any suitable number of electrode patterns may be cycled at any suitable rate, as discussed with reference to operation 810 of FIG. 8. Further, although specific treatment parameters are described for purposes of illustration, other suitable treatment parameters may be used in accordance with other embodiments and as readily recognizable by one skilled in the art. Accordingly, the example discussed with reference to FIGS. 14A to 14F should be considered as an illustrative example and not limiting in any way.

Mobile Cart

Systems for selectively applying electric fields to target areas include various components such as electrodes, a system control unit, and an imaging device. The various components may be provided in any suitable mechanical apparatus or system. In one embodiment, one or more of the components may be provided as a mobile unit such as a mobile cart. By providing components as a mobile unit, the system may advantageously be moved with relative ease to and/or between subjects or other elements for which it is desired to apply controlled voltages.

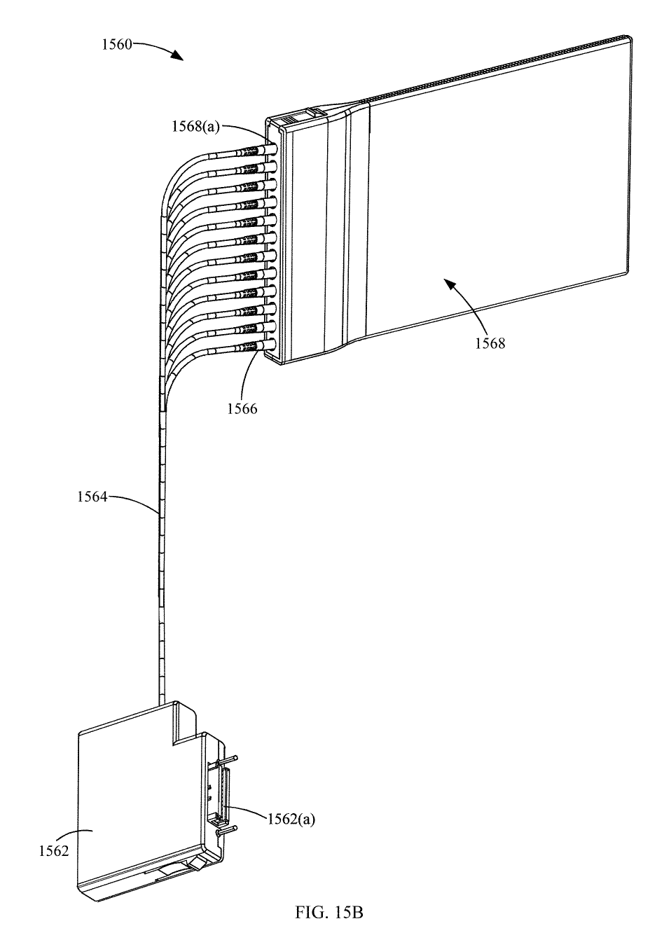

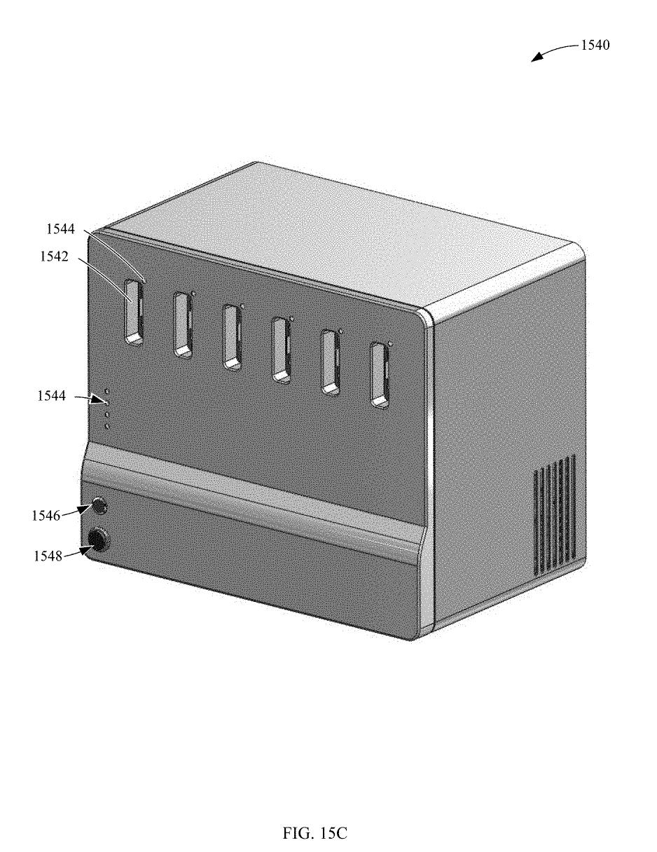

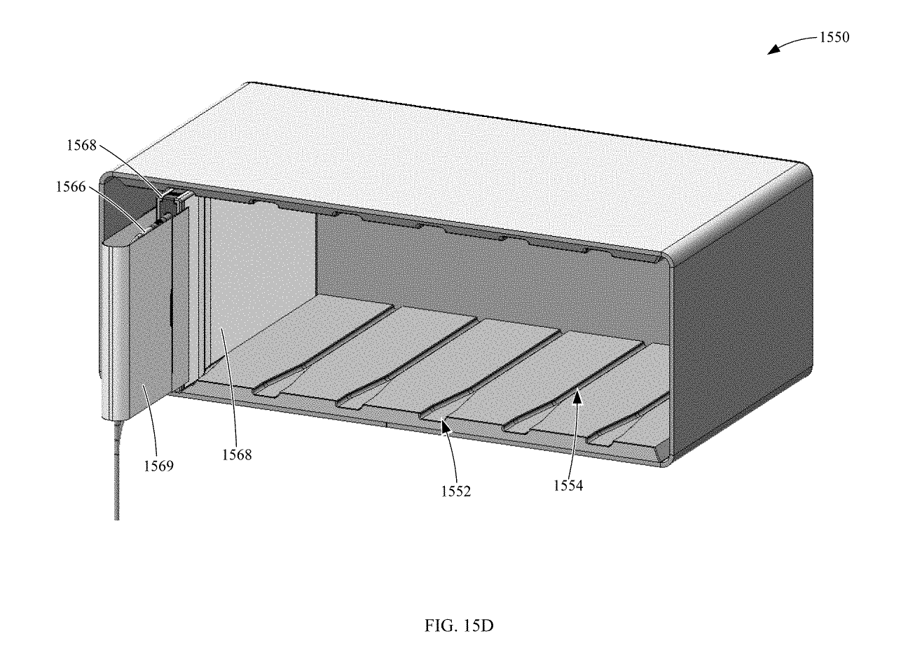

FIG. 15A illustrates a mobile cart 1500 including one or more components for selectively applying electric fields to target areas in accordance with an embodiment. Mobile cart 1500 includes a frame 1510 for supporting various elements, where frame 1510 is mounted on a base 1520 that includes moving elements 1522 such as wheels (or tracks, skis, belts, or other suitable elements for moving frame 1510) operable to move frame 1510. Mobile cart 1500 also includes a display device 1530 mechanically mounted to frame 1510 and operable to display information to a user such as a medical practitioner, and in some embodiments may also be operable to receive inputs from the user. For example, display device 1530 may be a touchscreen display. Display device 1530 may be the same as display device 130 discussed with reference to FIG. 1B.

Mobile cart 1500 may also include a controller 1540 mechanically mounted to frame 1510 which may include various components for controlling display device 1530 and one or more electrodes. For example, controller 1540 may include a processor, storage element, data acquisition card, amplifier board, etc. In one embodiment, controller 1540 may include computing device 120, amplifier board 140, isolation transformer 150, and/or power supply 160 discussed with reference to FIG. 1B.

Mobile cart 1500 may also include a cassette rack 1550 mechanically mounted to frame 1510. Cassette rack 1550 may be operable to receive part of a needle electrode assembly 1560. For example, needle electrode assembly 1560 may include a cassette connector 1562, one or more wires 1564, one or more electrodes 1566, and a cassette 1568. Cassette rack 1550 may be operable to receive cassette 1568, and controller 1540 may be operable to receive cassette connector 1562.

The components of mobile cart 1500 may be provided in any suitable arrangement for allowing a user to interact with display device 1530 and access electrodes 1566 to subsequently position electrodes 1566 near a target area. For example, display device 1530 may be provided at or near the top of frame 1510, cassette rack 1550 may be arranged below display device 1530, and controller 1540 may be arranged below cassette rack 1550.

FIG. 15B illustrates a cassette-based needle electrode assembly 1560 according to an embodiment. Electrode assembly 1560 includes cassette connector 1562, one or more wires 1564, one or more electrodes 1566, and cassette 1568. Needle electrode assembly 1560 may be similar to needle electrode assembly 170 discussed with reference to FIG. 1B and/or electrode assembly 200 discussed with reference to FIGS. 2A to 2F. In contrast to electrode assembly 200, however, in this embodiment cassette connector 1562 (similar to housing 230) does not include apertures 238 for receiving electrodes. Further, instead of being coupled to system control unit 108 via a cable assembly 145 that is couplable to interface 236(a), cassette connector 1562 includes a plug 1562(a) that may mechanically coupled directly to a corresponding receptacle of controller 1540. Accordingly, in this embodiment, cable assembly 145 may be omitted.

Various elements of electrode assembly 1560 may be the same as or similar to electrode assembly 200 discussed with reference to FIGS. 2A to 2F. For example, electrodes 1566 may be the same as electrodes 210, and wires 1564 may be the same as wires 220 and may include enlarged portions similar to enlarged portion 222. Further, cassette connector 1562 may have any suitable shape and be made of any suitable material, similar to housing 230, and may include various circuitry (e.g., electronics for calculating thermal measurements) similar to that discussed with reference to housing 230.

In some embodiments, electrode assembly 1560 may also include cassette 1568, which is operable to hold electrodes 1566. Cassette 1568 may include one or more apertures suitable sized and spaced to receive electrodes 1566. For example, apertures 1568a may be similar to apertures 238 discussed with reference to FIG. 2E.

FIG. 15C illustrates a controller 1540 according to an embodiment. Controller 1540 includes one or more apertures 1542 sized and shaped to receive cassette connectors 1562 (FIG. 15A and FIG. 15B). Cassette connectors 1562 may engage apertures 1542 using any suitable mechanical connection mechanism. For example, cassette connectors 1562 may engage apertures 1542 via retaining snaps formed in one or more of a cassette connector 1562 and controller 1540. As a result of engaging cassette connector 1562 with controller 1540, electrodes 1566 coupled to cassette connector 1562 may be electrically coupled to components of controller 1540 so that controller 1540 may subsequently control voltages and/or currents applied to electrodes 1566 and, in some embodiments, may acquire information (e.g., temperature information) via electrodes 1566.

Controller 1540 may include one or more of a variety of components other than apertures 1542. For example, controller 1540 may include status indicators 1544 for displaying various status information concerning the operation of controller 1540 and/or connectivity of a cassette connector 1562 to controller 1540, a power switch 1546 for activating and deactivating controller 1540, and an emergency stop button 1548 for disabling controller 1540 and/or causing controller 1540 to stop providing voltage and/or current to electrodes 1566.

FIG. 15D illustrates a cassette rack 1550 for receiving one or more cassettes 1568. Cassette rack 1550 includes one or more cassette lead-in's 1552 formed on one or more inner surfaces of cassette rack 1550. Lead-in's 1552 are each shaped to receive a cassette 1568 and direct cassette 1568 to channels 1554 formed in the inner surfaces of cassette rack 1550. Channels 1554 are sized and shape to receive cassettes 1568 and apply a resilient holding force to cassettes 1568 such that cassettes 1568 may be held in place by cassette rack 1550.

As shown in FIG. 15D, cassette rack 1550 may receive a part of electrode assembly 1560, such as cassette 1568. Further, in some embodiments and as shown in FIG. 15D, electrode assembly 1560 may also include a drape 1569. Drape 1569 is sized and shaped to surround electrodes 1566 when electrodes 1566 are disposed in cassette 1568. Drape 1569 may be made of any suitable material for protecting electrodes 1566 from users and users from electrodes 1566. In one embodiment, drape 1569 may be made of an insulating material.

Mobile cart 1500 in certain embodiments is an apparatus for providing a mobile system via which electric fields may be selectively applied to target areas, and may include various components such as a frame, base, display device, controller, and electrodes. However, it will be appreciated by those of ordinary skill in the art that the mobile cart could operate equally well by having fewer or a greater number of components than are illustrated in FIGS. 15A to 15D. For example, instead of including cassette rack 1550 and electrode assembly 1560, mobile cart 1500 may include a wired electrode assembly such as electrode assembly 200 (FIGS. 2A to 2F). Thus, the depiction of mobile cart 1500 in FIGS. 15A to 15D should be taken as being illustrative in nature, and not limiting to the scope of the disclosure.

The various systems, mobile carts, and components thereof may be used in one or more of a variety of fashions to apply electromagnetic fields to target areas. In one embodiment, a display device such as display device 130 discussed with reference to FIG. 1B and/or display device 1530 discussed with reference to FIG. 15A may be used to provide information regarding a treatment to a practitioner and, in some cases, may also be used to receive information from the practitioner. For example, the display device may display various configuration information for configuring a treatment, such as information requesting a treatment period, a desired electrode temperature, a desired electrode placement, etc., various status information indicating a status of the treatment, such as a current treatment time, electrode temperature, indication of a relationship between the current electrode temperature and a desired electrode temperature, etc., and/or other suitable information for facilitating the application of electromagnetic fields to target areas. By way of the display device and its interface with a user, a controller such as controller 1540 may be operated to control electromagnetic fields applied to a treatment area via electrodes such as electrodes 1566. The controller may use the configuration information, in conjunction with one or more pre-programmed electrode control algorithms, in controlling the electromagnetic fields.

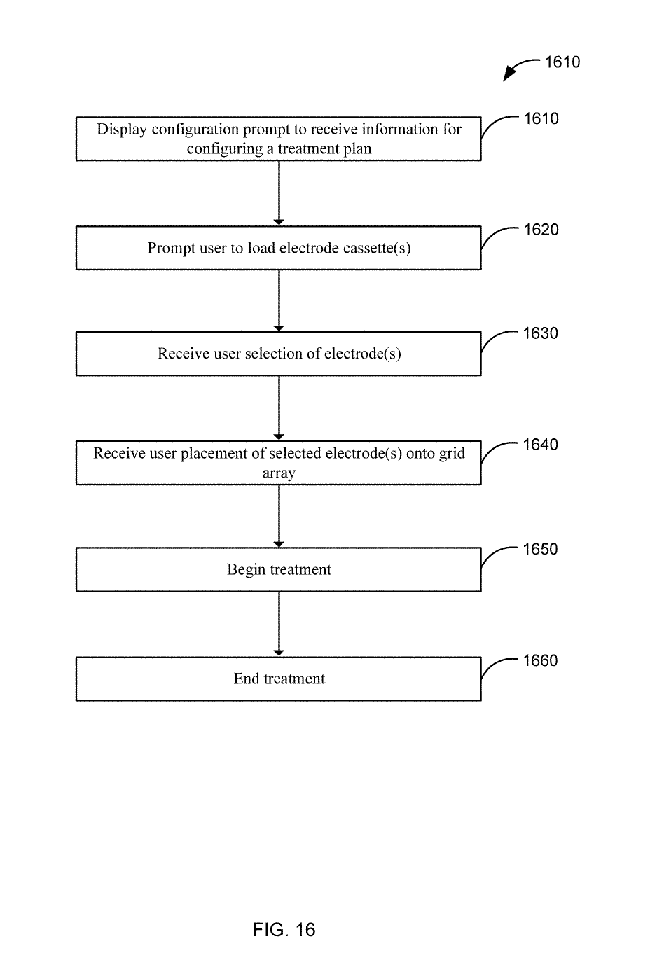

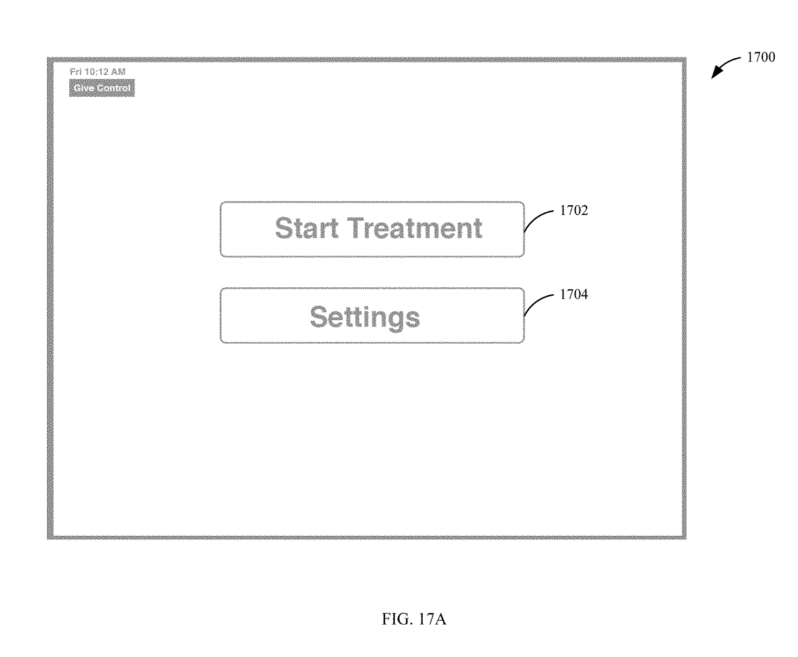

FIG. 16 shows a method 1600 for facilitating treatment of a target area. In operation 1610, a configuration prompt is displayed to solicit information for configuring a treatment plan. The configuration prompt may be displayed via, e.g., display device 1530 (or display device 130), and may prompt a user to enter various configuration information which may then subsequently be stored and used by other components of mobile cart 1500 (or system control unit 108). The configuration information may be any suitable information for configuring a treatment plan, such as a case number or other identifier associated with a particular treatment, a desired treatment period, a maximum electrode temperature, a minimum electrode voltage, and a maximum electrode voltage.

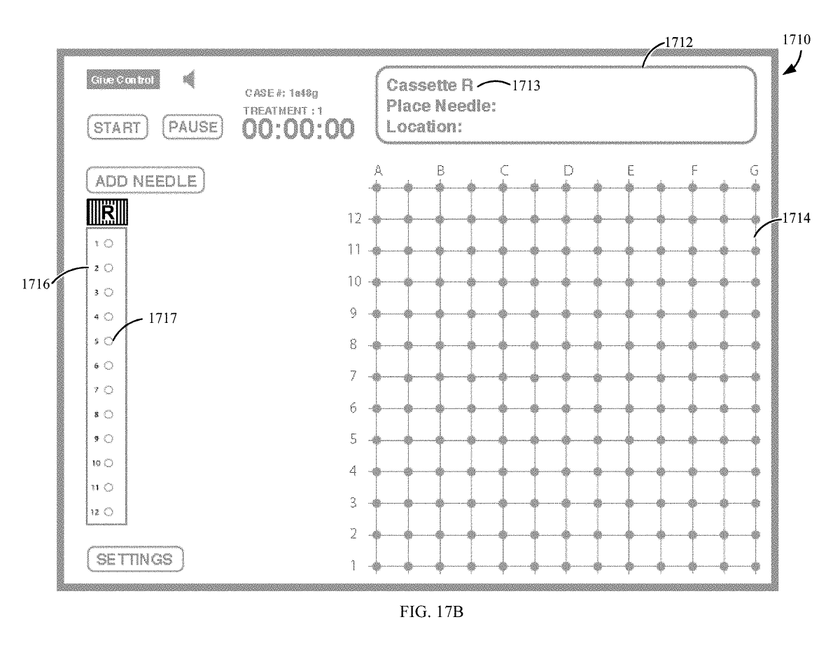

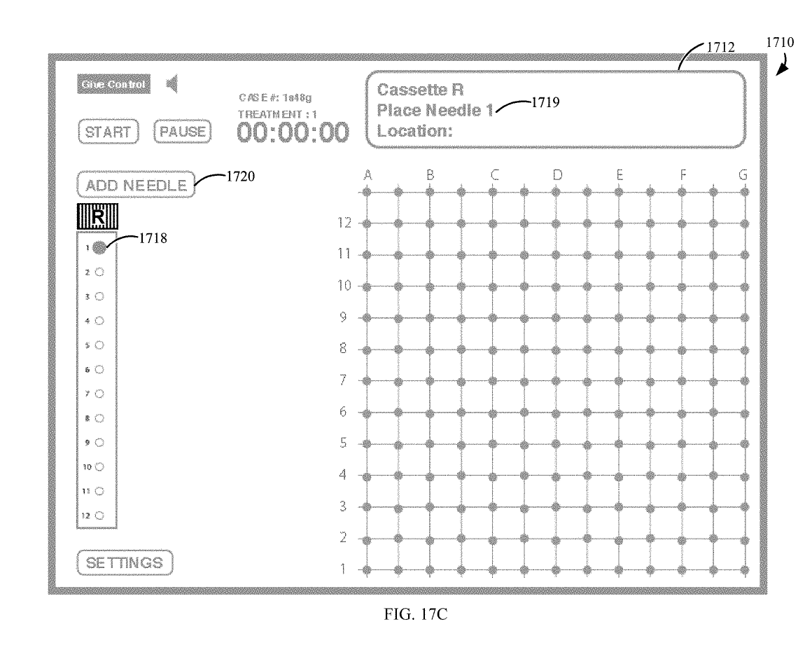

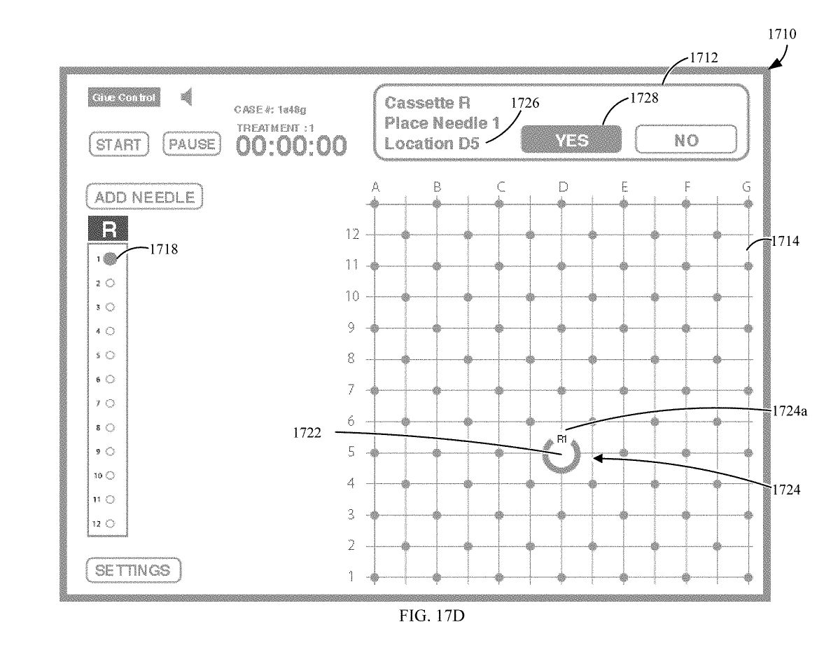

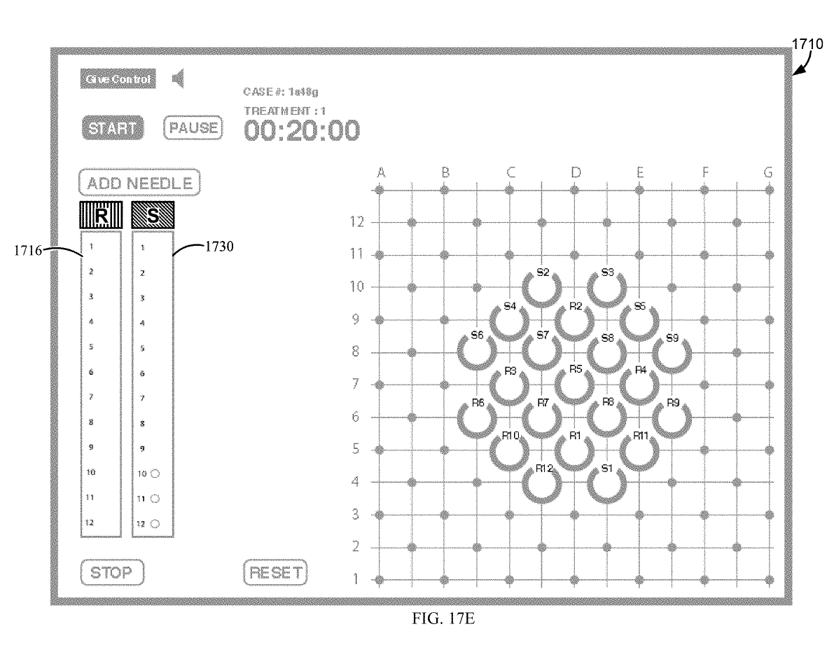

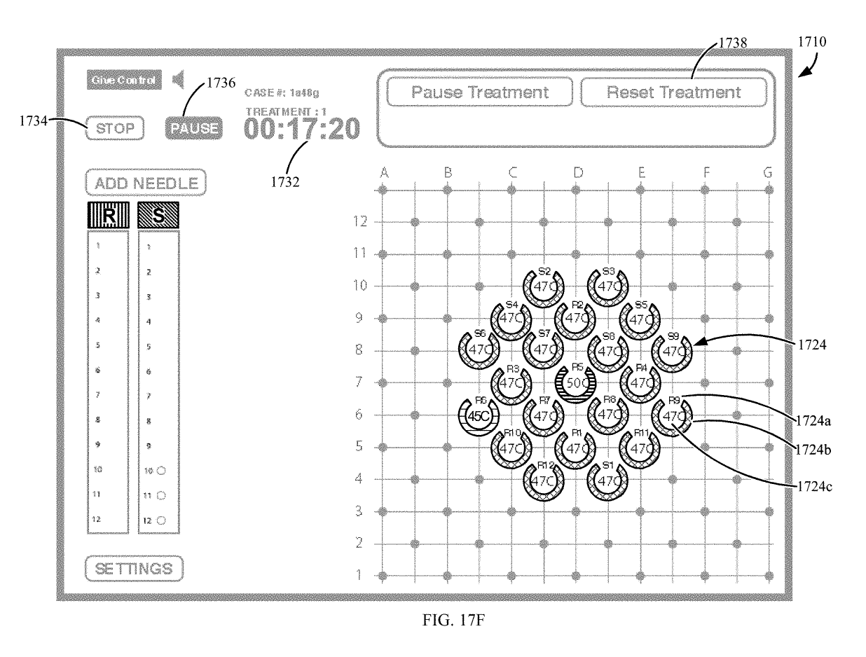

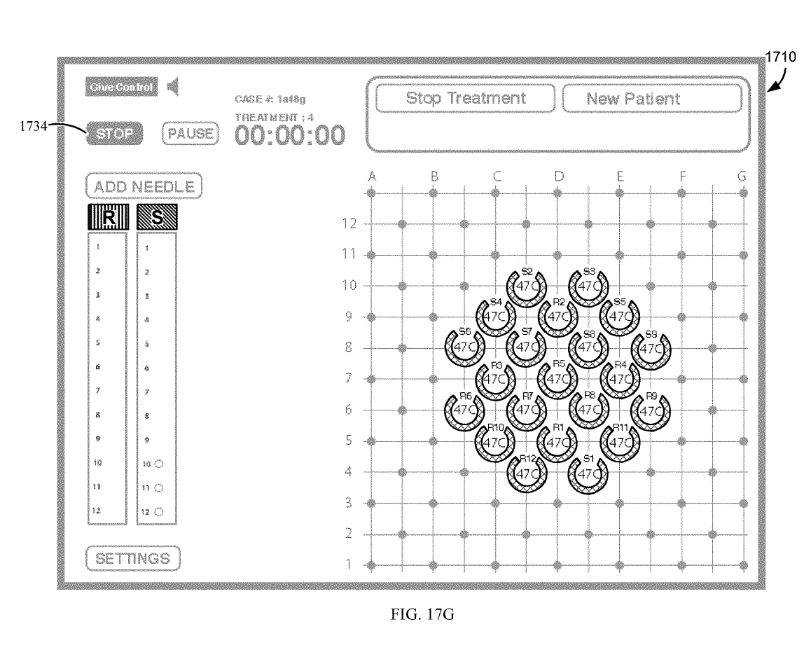

Turning briefly to FIG. 17A, FIG. 17A shows a user interface 1700 for displaying a configuration prompt according an embodiment. User interface 1700 may, in some embodiments, be used to facilitate operation 1610. User interface 1700 may be displayed on a display device such as display device 1530, and include a start treatment button 1702 and a settings button 1704. In response to user actuation of settings button 1704, user interface 1700 may display one or more additional prompts for receiving configuration information. Some additional prompts are subsequently discussed with reference to FIGS. 17B to 17D. In response to user actuation of start treatment button 1702, electromagnetic fields may be applied to a treatment area in accordance with configuration information either received by a user or provided as a default configuration.

Returning to FIG. 16, in operation 1620, a user is prompted to load one or more electrode cassettes. For example, a user may be prompted to load one or more cassettes 1568 into cassette rack 1550. In response to loading a cassette 1568 into cassette rack 1550, an image or other graphical representation of the cassette may be displayed to the user, where the image includes a number of selectable electrodes corresponding to a number of electrodes included in cassette 1568.

For example, FIG. 17B shows a user interface 1710 for a loaded cassette. User interface 1710 may be displayed at any suitable time. For example, user interface 1710 may be displayed in response to a cassette 1568 being loaded into cassette rack 1550, or in response to a user selecting settings button 1704 (FIG. 17A). User interface 1710 includes a dialogue box 1712 including a cassette identifier 1713 that displays a unique identifier associated with cassette 1568. User interface 1710 also includes a grid array 1714 similar to previously discussed grid array 772 (e.g., FIG. 7E). User interface 1710 may further include a cassette representation 1716 that is a digital representation of received cassette 1568, where cassette representation includes a number of electrode representations 1717 that correspond to electrodes 1566 mounted in cassette 1568. User interface 1710 may, by way of its configuration, inherently prompt a knowledged user to load one or more cassettes, or in some embodiments may display information explicitly requesting the user to load one or more cassettes.

In response to a cassette 1568 being loaded into cassette rack 1550, controller 1540 may perform a variety of other processing. For example, controller 1540 may perform a test on the receive cassette or instruct the received cassette to perform a self test, so as to test cassette temperatures, sorts, expiration dates, etc. In the event that the test identifies one or more problems with cassette 1568, one or more prompts may be displayed to the user via display device 1530 indicating such problems.

In operation 1630, a user selection of an electrode to be placed is received. For example, a user may select a digital representation of one of the electrodes of the received cassette. A purpose of such a selection may be to subsequently place the digital representation of the electrode into a particular location on grid array 1714, and/or configure one or more other aspects of the selected electrode, such as desired temperature, maximum voltage, minimum voltage, etc.

With reference to FIG. 17C, FIG. 17C shows the user interface of FIG. 17B with a user-selected cassette electrode 1718. According to one embodiment, a user may select cassette electrode 1718 by touching display device 1530. In response to user selection of cassette electrode 1718, dialogue box 1712 may display an electrode identifier 1719 identifying the electrode selected by the user. In the embodiment shown in FIG. 17C, the first electrode of cassette R is selected. In some embodiments, user interface 1710 may allow selection of an electrode only after a user has submitted a request to add an electrode. For example, user interface 1710 may allow selection of cassette electrode 1718 only after a user has actuated add needle button 1720.

In operation 1640, a user placement of the selected electrode onto grid array 1714 is received. For example, a user may choose to place the electrode selected from the cassette onto a location of grid array 1714. Grid array 1714 should generally correspond to apertures of a template such as template 500, and placement of the graphical representations of electrodes onto grid array 1714 should correspond with placement of the actual electrodes into corresponding apertures of template 500. By providing such a correspondence, user interface 1710 may subsequently provide a graphical representation of actual electrodes disposed in or around a target area.

Turning briefly to FIG. 17D, FIG. 17D shows the user interface of FIG. 17C with a user selected cassette electrode 1718 having been placed at a node 1722 of grid array 1714. In some embodiments, a user may select the location at which electrode 1718 is to be placed by touching the desired location of user interface 1710 subsequent to selecting the desired electrode to be placed. The graphical representation of the placed electrode 1724 may include a variety of information concerning the corresponding electrode, similar to that discussed with reference to FIG. 7. In one embodiment, placed electrode 1724 includes an electrode identifier 1724a identifying the placed electrode. Electrode identifier 1724a may include any suitable identification information for uniquely identifying the placed electrode, and in one embodiment includes a combination of the cassette identifier (e.g., “R”) and the electrode number (e.g., “1”). In some cases, dialogue box 1712 may include location information 1726 identifying the location in grid array 1714 in which the selected electrode has been placed, and may include a confirmation prompt 1728 requesting confirmation from the user that the selected electrode placement location is desired.

When it is desired to place a plurality of electrodes, operations 1630, and 1640 may be repeated for each electrode of each loaded cassette, and when it is desired to load multiple cassettes operation 1620 may be repeated. In some embodiments, a plurality of cassettes may be loaded and the electrodes from some or all of the cassettes configured. FIG. 17E shows a user interface 1710 in which a plurality of electrodes from two cassettes have been placed. Cassette representation 1716 may be referred to as “cassette R”, and cassette representation 1730 may be referred to as “cassette S”. As shown in FIG. 17E, a number of electrodes may be placed from each cassette into a desired pattern. The desired pattern may be any suitable pattern desired by a practitioner to apply electromagnetic fields to a particular treatment area. In some embodiments, all of the electrodes in each cassette may be placed, while in other embodiments, less than all of the electrodes in each cassette may be placed. For example, as shown in FIG. 17E, only twenty-one of the twenty-four available electrodes are placed, where all electrodes of cassette R are placed but only nine electrodes from cassette S are placed. As a result, controller 1540 may cause electromagnetic fields to be applied only to those placed electrodes, i.e., only to those twenty-one placed electrodes.

In operation 1650, treatment begins. Treatment begins by controller 1540 using any received configuration information, together with any suitable configuration defaults and pre-programmed electrode control algorithms, to cause electrodes 1566 which have been digitally placed via user interface 1710 to generate electromagnetic fields. The pre-programmed electrode control algorithms may include any of those previously discussed, such as any or all of those discussed with reference to FIGS. 8 to 13C. During treatment, various status information concerning the treatment may be displayed to the user, such as remaining treatment time, electrode temperatures, etc., and options may be presented to the user to stop, pause, and/or reset treatment.

For example, FIG. 17F shows the user interface of FIG. 17E after treatment has begun. User interface 1710 includes a number of placed electrodes 1724, where each electrode includes electrode identifier 1724a (similar to electrode identifier 786 of FIG. 7F), relative temperature indicator 1724b (similar to relative temperature indicator 784 of FIG. 7F), and current temperature 1724c (similar to current temperature 778 of FIG. 7F). Additional or other electrode status information such as that discussed with reference to FIG. 7F may also or alternatively be included in user interface 1710. User interface 1710 may also include various configuration information, such as a remaining treatment time indicator 1732 indicating an amount of time remaining in a current treatment. In some embodiments, user interface 1710 may also include treatment options such as a stop button 1734, actuation of which causes a treatment to stop, a pause button 1736, actuation of which causes a treatment to be paused, and/or a reset treatment button 1738, actuation of which causes a treatment to be reset (i.e., restarted).

In operation 1660, treatment ends. Treatment ends when the treatment period expires, controller 1540 receives a user input to stop, pause, or otherwise terminate treatment, and/or controller 1540 detects one or more fault conditions such as an electrode short, significant overheating, a hardware or a software failure in any of the components of the system, etc. Once treatment ends, display device 1530 may prompt the user to perform additional tasks, such as removing needles from the template, place sharp objects in containers, unplug connections, remove templates the electrode guide, power down the system, etc.

FIG. 17G shows the user interface of FIG. 17F upon completion of a treatment. According to this embodiment, treatment ended as a result of expiration of the treatment time. In some cases, the status information of each of the electrodes may be displayed until a user input is received indicating an end of the treatment. For example, upon receiving a user selection of stop button 1734, other information such as prompts for the user to perform additional tasks may be displayed via display device 1530.

In accordance with some embodiments, a treatment may include a number of treatment cycles, where each treatment cycle may be the same or different than a previous treatment cycle. For example, a treatment may include a first treatment cycle in which electrodes are controlled to heat a target area for a set duration, and a second treatment cycle in which the electrodes are controlled to heat the target area for the same or a different duration at the same or a different temperature as configured in the first treatment cycle. Controller 1540 may sequentially execute the treatment cycles which may be preconfigured or configured in sequence at the end of a previous treatment cycle. Multiple preconfigured treatment cycles that are sequentially executed may advantageously be used in situations where it is desired to heat treatment volumes at different depths or at otherwise different locations within the volume. For example, electrodes may be disposed in a treatment volume (e.g., a prostate) a first depth in the treatment volume. Upon execution of the first treatment cycle, the electrodes may apply electromagnetic fields at the first depth in the treatment volume. Once the first treatment cycle is complete, the treatment may be paused, whereby controller 1540 prevents electric fields to be applied via the electrodes or otherwise disables the electrodes. The electrodes may then be relocated to a second depth in the treatment volume. Upon relocation of the electrodes to the second depth, the second treatment cycle may be executed. In such a fashion, a three-dimensional volume may be effectively treated.

It should be appreciated that the specific operations illustrated in FIG. 16 provide a particular method for facilitating treatment of a target area, according to certain embodiments of the present invention. Other sequences of operations may also be performed according to alternative embodiments. For example, alternative embodiments of the present invention may perform the operations outlined above in a different order. Moreover, the individual operations illustrated in FIG. 16 may include multiple sub-operations that may be performed in various sequences as appropriate to the individual operation. Furthermore, additional operations may be added or existing operations removed depending on the particular applications. One of ordinary skill in the art would recognize and appreciate many variations, modifications, and alternatives.

Further, user interfaces 1700 and 1710 in certain embodiments are interfaces for facilitating treatment of a target area, and may include various elements such as a dialogue box 1712, grid array 1714, and cassette representation 1716. However, it will be appreciated by those of ordinary skill in the art that the user interface could operate equally well by having fewer or a greater number of components than are illustrated in FIGS. 17A to 17G. For example, user interfaces 1700 and 1710 may include any or all, or be replaced with, the interfaces described in with reference to any or all of FIGS. 7A to 7F. Thus, the depiction of user interfaces 1700 and 1710 in FIGS. 17A to 17G should be taken as being illustrative in nature, and not limiting to the scope of the disclosure.

It should be understood that the examples and embodiments described herein are for illustrative purposes only and that various modifications or changes in light thereof will be suggested to persons skilled in the art and are to be included within the spirit and purview of this application and scope of the appended claims. Further, numerous different combinations are possible, and such combinations are considered part of the present invention.

For example, user interface 700 may provide any suitable mechanism for receiving information from an operator. With reference to FIG. 7B, while fields such as those illustrated with the treatment parameter values of treatment parameter element 710 are illustrated, other input mechanisms are also within the scope of this application, such as radial buttons, drop-down menus, push buttons, icons, etc. Similarly, with reference to FIG. 7D, while electrodes may be controlled by dragging and dropping an electrode polarity selector 752 to a location on electrode status element 770, other control techniques are also within the scope of this application, such as field entry, radial buttons, drop-down menus, push buttons, icons, etc.

For another example, user interface 700 may display information in any suitable arrangement. While user interface 700 is discussed as separate elements for displaying and inputting specific information, such as treatment parameter element 710, patient information element 730, electrode control element 750, and electrode status element 770, these elements may be integrated, or partially integrated, and the information displayed therefrom and input thereto may be displayed and input onto the elements as described or onto different elements. For example, while start button 722 is illustrated as being a part of treatment parameter element, start button 722 may be additionally or alternatively part of a different element, such as electrode status element 770.

For yet another example, instead of generating and controlling current flows between electrodes, current flows could be generated and controlled between electrodes and a return pad. That is, with reference to FIG. 1A, a conductive pad may be provided separate from electrodes 102, such as outside of the patient's body. A current flow may then be generated between electrodes 102 and the conductive pad, for heating tissue located between electrodes 102 and the conductive pad. Electrode control techniques similar to those discussed with respect to, e.g., FIG. 8, may then be applied to average tissue heating over some or all of the tissue located between electrodes 102 and the conductive pad, and reduce localized heating of individual electrodes 102. For another example, instead of controlling current flows between electrodes, heating of individual electrodes (e.g., by charging a resistive component of the electrodes) could be controlled. That is, electrode control techniques similar to those discussed with respect to, e.g., FIG. 8, may be applied to average tissue heating over some or all of the tissue located proximate to electrodes 102, and reduce localized heating of individual electrodes 102.

In certain embodiments, methods and structures as described herein have been demonstrated as remarkably effective in delivering fields to a target tissue while more precisely controlling the resulting temperature applied to the tissue (e.g., controlled tissue heating). Selectively controlling the electromagnetic fields generated by a plurality of electrodes with a corresponding control of applied temperature or heating as described herein can offer several advantages. In accordance with various embodiments described herein, voltages applied to electrodes, and accordingly the current paths established between electrodes, can be specifically controlled, resulting in an unprecedented temperature control of target volumes in which the electrodes are disposed.

Target tissue heating involving methods and structures described herein is not limited to any particular target temperature or temperature range. Delivery of electromagnetic fields as described herein, for example, may include heating of tissue from no discernable increase in tissue temperature above baseline (e.g., body temperature, such as normal human body temperature of about 37 degrees C.) to temperatures inducing indiscriminate, heat-mediated tissue destruction (e.g., tissue necrosis, protein cross-linking, etc.). For example, target tissue heating temperatures may include increases of target tissue from about 0 to about 5, 10, 20, 30 degrees C. (or higher) above baseline, as well as any temperature increment therebetween.

In some embodiments, current delivery may be selected to elicit mild tissue heating, such that target tissue is heated a few degrees above baseline or body temperature, such as 0.1 to about 10 (or more) degrees Celsius above baseline or body temperature (e.g., 1, 2, 3, 4, 5, 6, 7, 8, 9, 10, 11, 12, etc. degrees Celsius above baseline). Such mild heating and/or accurate temperature control through a target volume can be particularly advantageous in applications where it is desired to destroy cancerous cells while minimizing damage to nearby healthy cells. For example, mild tissue heating may be selected such that current delivery elicits preferential disruption or destruction to cancerous cells in a target tissue (e.g., target tissue volume) compared to non-cancerous cells in the target tissue.

As described above, methods and structures described herein further allow for more precise control of the temperatures or temperature ranges of the target tissue or heating elicited in the target tissue with delivery of electromagnetic fields. Thus, target temperatures can include a target range or selected/expected deviation from the target temperatures. For example, tissue heating temperatures or ranges can include a modest deviation from a target, and will typically be less than a few degrees Celsius, and in some instances less than about 1 degree Celsius (e.g., 0.001 to about 1 degree Celsius). For example, actual heating may be from +/− about 0.001 to about 10 degrees Celsius, or any increment therebetween.

In some of the embodiments described, desired voltages, maximum voltages, minimum voltages, and/or voltage ranges may be defined. For example, a maximum voltage may be 3V, 4V, 5V, in the range from 3V to 5V, 0V to 5V, −5V to 5V, or less than −5V or greater than 5V. By setting such maximum voltages, the control algorithms operate to achieve the desired temperature by setting the appropriate voltage differentials so as to establish appropriate current flows, all without exceeding the set voltage levels. Such selective voltage control is particularly advantageous in applications where excess voltage levels or differentials may cause undesirable secondary effects. Similarly, by controlling maximum voltage ranges, then maximum current ranges are inherently imposed. In some embodiments, instead of a user providing maximum voltages or voltage ranges, a user may input maximum currents or current ranges, which has similar advantages to controlling the maximum voltage levels.

Throughout this description, reference may be made to selected or desired temperatures. Temperatures can be actually temperatures, predicted or calculated temperatures, or measured temperatures (e.g., directly or indirectly measured tissue temperatures). In some embodiments, such temperatures may correspond to the temperature of an electrode, subset of electrodes, or all electrodes disposed in a target volume. For example, electrode temperature may be acquired via a temperature sensor disposed in an electrode, such as temperature sensor 330 (FIG. 3B), but may also or alternatively be acquired via a temperature sensor disposed proximate the electrode or even outside of the target volume which the electrodes are disposed in (e.g., via remote thermal sensing). Accordingly, in other embodiments, the temperatures may correspond not to the temperature of an electrode, but rather to the temperature of tissue or a target area in contact with an electrode(s) or proximate an electrode(s). Further, the temperature may not be the actual temperature of the electrode or target volume, but rather, in some embodiments, could be an approximation or predicted temperature of the electrode or target volume. For example, the temperature of one electrode could be approximated by using a reading from a temperature sensor disposed in a proximate electrode. While not exact, the temperature of the proximate electrode may be a good approximation of the temperature of the electrode at issue as long as the electrodes are disposed close enough to each other.

While the present invention is described with particular reference to targeting prostate tissue or tissues in or proximate to the prostate of a patient, structures and methods described herein can be utilized for targeting various different tissues other than those of or proximate to a patient's prostate, and are not intended for limitation to any particular tissue or bodily location. For example, structures and methods of the present invention can be utilized for targeting various different tissues including cancerous cells of various tissue types and locations in the body, including without limitation breast, liver, lung, colon, kidney, brain, uterine, ovarian, testicular, stomach, pancreas, etc.

Although the description herein is provided in the context of applying voltages to target tissues, voltages may be applied to target areas of any suitable material. For example, voltages may be applied to metals, polymers, ceramics, or other types of material. The material may be solid, liquid, gaseous, or in any other suitable state.

Accordingly, the scope of the invention should be determined not with reference to the above description, but instead should be determined with reference to the pending claims along with their full scope or equivalents.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of controlling electric fields created by a plurality of electrodes, comprising:
repetitively applying multiple sets of voltages to at least some of a plurality of electrodes over a treatment period so as to heat a target tissue to a selected temperature or temperature range, the at least some of the electrodes being treatment electrodes, and the multiple sets of voltages including:
a first set of voltages that creates an electric potential difference between at least some adjacent pairs of the treatment electrodes; and
a second set of voltages that creates an electric potential difference between at least some adjacent pairs of the treatment electrodes for which an electric potential difference was not created while applying the first set of voltages,
wherein the multiple sets of voltages in combination create an electric potential difference between each adjacent pair of treatment electrodes.

2. The method of claim 1, wherein applying the second set of voltages removes an electric potential difference between at least one of the adjacent pairs of treatment electrodes that was created while applying the first set of voltages.

3. The method of claim 1, wherein applying the first set of voltages creates an electric potential difference between a first one of the treatment electrodes and one or more first adjacent treatment electrodes, and applying the second set of voltages creates an electric potential difference between the first one of the treatment electrodes and one or more second adjacent treatment electrodes different than the first adjacent treatment electrodes.

4. The method of claim 1, further comprising applying one or more additional sets of voltages to the treatment electrodes so that, together with application of the first set of voltages and the second set of voltages, a current flow between each adjacent pair of treatment electrodes is approximately the same.

5. The method of claim 1, wherein creating an electric potential difference includes one or more of:
providing an electrical voltage having a first polarity to a first electrode of a pair of treatment electrodes and an electrical voltage having a second polarity different than the first polarity to a second electrode of the pair of treatment electrodes;
providing an electrical voltage having a first phase to a first electrode of a pair of treatment electrodes and an electrical voltage having a second phase different than the first phase to a second electrode of the pair of treatment electrodes; and
providing an electrical voltage having a first amplitude to a first electrode of a pair of treatment electrodes and an electrical voltage having a second amplitude different than the first amplitude to a second electrode of the pair of treatment electrodes.

6. The method of claim 1, further comprising:
applying a feedback control loop controlling the electrical voltage provided to the treatment electrodes, wherein applying a feedback control loop includes, for each treatment electrode:
adjusting a voltage applied to the electrode based at least in part on one or more of:
a temperature difference for the electrode based on a temperature of an adjacent electrode; and
an estimate of a voltage at the electrode provided by one or more other electrodes.

7. The method of claim 6, further comprising, for each treatment electrode:
reading an electrode temperature of the treatment electrode;
reading an electrode temperature of one or more electrodes that are located adjacent to the treatment electrode; and
if a temperature of one of the adjacent electrodes is higher than the temperature of the treatment electrode, then adjusting the voltage applied to the treatment electrode based on the higher temperature.

8. The method of claim 6, further comprising, for each treatment electrode:
identifying voltages of adjacent electrodes;
adjusting the identified voltages based on a distance of the adjacent electrodes from the treatment electrode;
determining the average of the adjusted voltages; and
adjusting the voltage applied to the treatment electrode based on the average of the adjusted voltages.

9. A system for selectively generating electric fields, comprising:
a plurality of electrodes; and
a control unit including a storage medium and a computer processor, the storage medium having executable instructions stored thereon, wherein the computer processor is operable to execute the instructions so as to cause the control unit to perform operations including:
switching between unique electrode patterns so as to heat a target tissue to a selected temperature or temperature range, where each unique electrode pattern includes providing an electrical voltage to at least some of the electrodes, the at least some electrodes being treatment electrodes, and the electrical voltage being provided so as to generate a current flow between adjacent pairs of the treatment electrodes; and
applying a feedback control loop controlling the electrical voltage provided to the treatment electrodes based at least in part on one or more of:
a temperature difference for a treatment electrode based on a temperature of an adjacent treatment electrode; and
an estimate of a voltage at a treatment electrode provided by one or more other treatment electrodes.

10. The system of claim 9, further comprising:
a user interface device coupled to the control unit, the user interface operable to:
display a graphical representation of a plurality of electrodes, the graphical representation including one or more of a voltage of each electrode, a current of each electrode, and a temperature of each electrode; and
receive a user input selecting at least some of the plurality of electrodes to be electrically connected to a power source;
wherein the control unit is operable to apply a voltage to the selected electrodes.

11. The system of claim 10, wherein the graphical representation of the plurality of electrodes includes a plurality of electrode elements corresponding to the plurality of electrodes and arranged to correspond to a physical layout of the plurality of electrodes.

12. The system of claim 9, further comprising:
a plurality of flexible conductive wires corresponding to the plurality of electrodes, a first end of each wire being mechanically coupled to an end of an electrode; and
a housing for selectively receiving the plurality of electrodes, the housing including:
a side surface having apertures for receiving the plurality of electrodes; and
an interface mechanically coupled to a second end of the plurality of wires for providing an electrical coupling to the plurality of electrodes.

13. The system of claim 12, wherein the apertures are sized to receive the electrodes and are spaced apart from one another so as to electrically insulate the plurality of elongated electrodes from one another when the housing receives the electrodes.

14. The system of claim 9, further comprising:
a first electrode template having a plurality of apertures for receiving the plurality of electrodes;
a second electrode template having a plurality of apertures for receiving the plurality of electrodes; and
an adjustable template securing apparatus mechanically couplable to the first electrode template and the second electrode template, the adjustable template securing apparatus including:
a first template mount for supporting the first electrode template;
a second template mount for supporting the second electrode template; and
a distance adjustment element mechanically couplable to the first template mount and the second template mount for adjusting a distance between the first electrode template and the second electrode template,
wherein at least one of the first template mount and the second template mount is removable from the electrode template it supports.

15. The system of claim 14, wherein the electrode templates each include at least one securing element extending from a surface of the template, and the template mounts each include at least one cutout for receiving the at least one securing element of a corresponding template.

16. The system of claim 14, wherein the second template mount includes at least one aperture for receiving the distance adjustment element.

17. A control unit for controlling electric fields created by a plurality of electrodes, the control unit including a storage medium and a computer processor, the storage medium having executable instructions stored thereon, wherein the computer processor is operable to execute the instructions so as to cause the control unit to perform operations including:
applying a feedback control loop controlling an electrical voltage provided to at least some of a plurality of electrodes so as to heat a target tissue to a selected temperature or temperature range, the at least some electrodes being treatment electrodes, wherein applying a feedback control loop includes, for each treatment electrode:
adjusting a voltage applied to the electrode based at least in part on one or more of:
a temperature difference for the electrode based on a temperature of an adjacent electrode; and
an estimate of a voltage at the electrode provided by one or more other electrodes.

18. The control unit of claim 17, wherein adjusting a voltage includes:
reading an electrode temperature of the electrode;
reading an electrode temperature of one or more treatment electrodes that are located adjacent to the electrode; and
if a temperature of one of the adjacent electrodes is higher than the temperature of the electrode, then adjusting the voltage applied to the electrode based on the higher temperature.

19. The control unit of claim 17, wherein adjusting a voltage includes:
identifying voltages of adjacent electrodes;
adjusting the identified voltages based on a distance of the adjacent electrodes from the electrode;
determining the average of the adjusted voltages; and
adjusting the voltage applied to the electrode based on the average of the adjusted voltages.

20. The control unit of claim 17, wherein the computer processor is operable to execute the instructions so as to cause the control unit to perform operations further including:
repetitively applying multiple sets of voltages to the treatment electrodes, the multiple sets of voltages including:
a first set of voltages that creates an electric potential difference between at least some adjacent pairs of the treatment electrodes; and
a second set of voltages that creates an electric potential difference between at least some adjacent pairs of the treatment electrodes for which an electric potential difference was not created while applying the first set of voltages,
wherein the multiple sets of voltages in combination create an electric potential difference between each adjacent pair of treatment electrodes.
